(19) United States
(12) Patent Application Publication
(10) Pub. No.: US 2009/0036420 A1
(43) Pub. Date: Feb. 5, 2009

(54) BENZAMIDE DERIVATIVES AND THEIR USE FOR TREATING CNS DISORDERS

(76) Inventors: Guido Galley, Rheinfelden (DE); Katrin Groebke Zbinden, Liestal (CH); Roger Norcross, Olsberg (CH); Henri Stalder, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/180,566

(22) Filed: Jul. 28, 2008

(30) Foreign Application Priority Data
Aug. 2, 2007 (EP) 07113657.6

(51) Int. Cl. A61K 31/397; A61K 31/55; A61K 31/5377; A61K 31/5375; A61K 31/505; A61K 31/4545; A61K 31/445; A61K 31/4427; A61K 31/4439; A61K 31/44; A61K 31/426; A61K 31/421; A61K 31/415; A61K 31/382; A61K 31/165; A61K 31/34; C07D 413/02; C07D 233/08; C07D 401/02; C07D 265/30; C07D 239/02; C07D 211/68; C07D 211/32; C07D 405/02; C07D 213/56; C07D 275/02; C07D 263/34; C07D 231/02; C07D 207/46; C07D 205/02; C07D 337/08; C07D 307/02; C07C 323/41; C07C 233/88; A61P 25/18; A61P 25/28; A61P 25/24; A61P 25/16

(52) U.S. Cl. 514/210.2; 514/618; 564/162; 564/184; 514/617; 544/159; 514/237.8; 544/165; 548/567; 548/375.1; 514/406; 514/428; 514/331; 546/234; 544/131; 514/235.5; 514/374; 548/236; 546/275.4; 514/341; 544/365; 514/253.13; 514/355; 546/337; 548/214; 514/372; 544/326; 514/256; 514/210.01; 548/950; 546/268.1; 514/318; 546/194; 540/604; 514/217.12; 546/282.1; 514/336; 514/471; 549/496; 549/9; 514/431

(57) ABSTRACT

The present invention relates to methods of treating CNS disorders with a compound of formula I

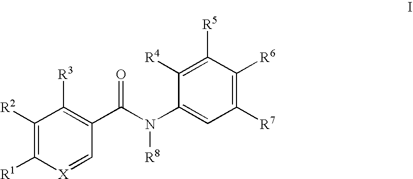

wherein
X, R¹, R², R³, R⁴, R⁵, R⁶, R⁷, and R⁸ are as defined in the specification and pharmaceutically acceptable acid addition salts thereof.

### PRIORITY TO RELATED APPLICATION(S)

This application claims the benefit of European Patent Application No. 07113657.6, filed Aug. 2, 2007, which is hereby incorporated by reference in its entirety.

### BACKGROUND OF THE INVENTION

The classical biogenic amines (serotonin, norepinephrine, epinephrine, dopamine, histamine) play important roles as neurotransmitters in the central and peripheral nervous system [Deutch, A. Y. and Roth, R. H. (1999) Neurotransmitters. In Fundamental Neuroscience (2^{nd} edn) (Zigmond, M. J., Bloom, F. E., Landis, S. C., Roberts, J. L, and Squire, L. R., eds.), pp. 193-234, Academic Press]. Their synthesis and storage, as well as their degradation and reuptake after release are tightly regulated. An imbalance in the levels of biogenic amines is known to be responsible for the altered brain function under many pathological conditions [Wong, M. L. and Licinio, J. (2001) Research and treatment approaches to depression. Nat. Rev. Neurosci. 2, 343-351; Carlsson, A. et al. (2001) Interactions between monoamines, glutamate, and GABA in schizophrenia: new evidence. Annu. Rev. Pharmacol. Toxicol. 41, 237-260; Tuite, P. and Riss, J. (2003) Recent developments in the pharmacological treatment of Parkinson's disease. Expert Opin. Investig. Drugs 12, 1335-1352; and Castellanos, F. X. and Tannock, R. (2002) Neuroscience of attention-deficit/hyperactivity disorder: the search for endophenotypes. Nat. Rev. Neurosci. 3, 617-628]. A second class of endogenous amine compounds, the so-called trace amines (TAs) significantly overlap with the classical biogenic amines regarding structure, metabolism and subcellular localization. The TAs include p-tyramine, β-phenylethylamine, tryptamine and octopamine, and they are present in the mammalian nervous system at generally lower levels than classical biogenic amines [Usdin, E. and Sandler, M. eds. (1984), Trace Amines and the brain, Dekker.]. Their dysregulation has been linked to various psychiatric diseases like schizophrenia and depression [Lindemann, L. and Hoener, M. (2005) A renaissance in trace amines inspired by a novel GPCR family. Trends in Pharmacol. Sci. 26, 274-281] and for identifying and testing for the therapeutic effect of a compound in treating and preventing disorders comprising depression, anxiety disorders, bipolar disorder, attention deficit hyperactivity disorder, stress-related disorders, psychotic disorders such as schizophrenia, neurological diseases such as Parkinson's Disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders [Branchek, T. A. and Blackburn, T. P. (2003) Trace amine receptors as targets for novel therapeutics: legend, myth and fact. Curr. Opin. Pharmacol. 3, 90-97; and Premont, R. T. et al. (2001) Following the trace of elusive amines. Proc. Natl. Acad. Sci. U.S.A. 98, 9474-9475].

For a long time, TA-specific receptors had only been hypothesized based on anatomically discrete high-affinity TA binding sites in the CNS of humans and other mammals [Mousseau, D. D. and Butterworth, R. F. (1995) A high-affinity [3H] tryptamine binding site in human brain. Prog. Brain Res. 106, 285-291; and McCormack, J. K. et al. (1986) Autoradiographic localization of tryptamine binding sites in the rat and dog central nervous system. J. Neurosci. 6, 94-101]. Accordingly, the pharmacological effects of TAs were believed to be mediated through the well known machinery of classical biogenic amines, by either triggering their release, inhibiting their reuptake or by “crossreacting” with their receptor systems [Premont, R. T. et al. (2001) Proc. Natl. Acad. Sci. U.S.A. 98, 9474-9475; Dyck, L. E. (1989) Release of some endogenous trace amines from rat striatal slices in the presence and absence of a monoamine oxidase inhibitor. Life Sci. 44, 1149-1156; and Parker, E. M. and Cubeddu, L. X. (1988) Comparative effects of amphetamine, phenylethylamine and related drugs on dopamine efflux, dopamine uptake and mazindol binding. J. Pharmacol. Exp. Ther. 245, 199-210]. This view changed significantly with the recent identification of several members of a novel family of GPCRs, the trace amine associated receptors (TAARs) [Lindemann, L. and Hoener, M. (2005) Trends in Pharmacol. Sci. 26, 274-281; and Lindemann, L. et al. (2005) Trace amine associated receptors form structurally and functionally distinct subfamilies of novel G protein-coupled receptors. Genomics 85, 372-385]. There are 9 TAAR genes in human (including 3 pseudogenes) and 16 genes in mouse (including 1 pseudogene). The TAAR genes do not contain introns (with one exception, TAAR2 contains 1 intron) and are located next to each other on the same chromosomal segment. The phylogenetic relationship of the receptor genes, in agreement with an in-depth GPCR pharmacophore similarity comparison and pharmacological data suggest that these receptors form three distinct subfamilies [Lindemann, L. and Hoener, M. (2005) Trends in Pharmacol. Sci. 26, 274-281; and Lindemann, L. et al. (2005) Genomics 85, 372-385]. TAAR1 is in the first subclass of four genes (TAAR1-4) highly conserved between human and rodents. TAs activate TAAR1 via Gαs. Dysregulation of TAs was shown to contribute to the aetiology of various diseases like depression, psychosis, attention deficit hyperactivity disorder, substance abuse, Parkinson's disease, migraine headache, eating disorders, metabolic disorders and therefore TAAR ligands have a high potential for the treatment of these diseases.

In conclusion, based on biochemical and behavioral data, the compounds having an affinity with TAAR ligands are expected to be a suitable drug candidate for the CNS disorders, such as depression, anxiety disorders, bipolar disorder, attention deficit hyperactivity disorder (ADHD), stress-related disorders, psychotic disorders, schizophrenia, neurological diseases, Parkinson's disease, neurodegenerative disorders, Alzheimer's disease, epilepsy, migraine, hypertension, substance abuse and metabolic disorders, eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders; in particular such as anxiety, depression, bipolar disorders, Parkinson's disease, schizophrenia and pain.

Meanwhile, focusing on the compound, there synthesized and reported numerous Phenyl-benzamide derivatives and N-Phenyl-nicotinamide derivatives. Among them, some of the documents referred to their possibilities for the treatment of a CNS disorder [Clitherow, J. W. et al. (1994) J. Med. Chem. 37(15), 2253-2257; WO 97/03967; WO 99/65449; WO 02/053544; WO 02/059080 and U.S. 2003/0105135 A1; However, it is still uncertain what sort of the compounds are suitable for the treatment of the CNS disorders.

### SUMMARY OF THE INVENTION

The present invention provides a method for the treatment of a CNS disorder selected from the group consisting of depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders by administering to an individual a therapeutically effective amount of a compound of formula I

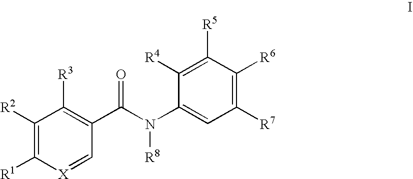

wherein
R¹ is hydrogen, halogen, lower alkyl, lower alkyl substituted by halogen, cycloalkyl, lower alkoxy, NO₂, —(CH₂)ₒS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O)O-lower alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl; or
R¹ and R² together with the corresponding C-atoms form a ring comprising —CH═CH—CH═CH-or —S—(CH₂)₄—;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR′R″, oxazol-5-yl or halogen;
R⁵ and R⁶ together with the corresponding C-atoms form a ring comprising —CH═CH—CH═CH—;
R⁸ is hydrogen or lower alkyl;
X is —C(R⁹)═ or —N═;
R⁹ is hydrogen, lower alkoxy, NO₂ or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ—O-lower alkyl;
n is 0, 1, 2 or 3, and
o is 0 or 1,
or a pharmaceutically acceptable acid addition salt thereof.

The present invention also relates to novel compounds of formula IA and IB.

The invention includes all sterioisomeric forms, including individual diastereoisomers and enantiomers of the compound of formula I as well as racemic and non-racemic mixtures thereof.

Compounds of formula I have a good affinity to the trace amine associated receptors (TAARs), especially for TAAR1. The compounds are useful for the treatment of depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

The preferred indications of the present invention are depression, psychosis, Parkinson's disease, schizophrenia, anxiety and attention deficit hyperactivity disorder (ADHD).

### DETAILED DESCRIPTION OF THE INVENTION

The following definitions of the general terms used in the present description apply irrespective of whether the terms in question appear alone or in combination. It must be noted that, as used in the specification and the appended claims, the singular forms “a”, “an,” and “the” include plural forms unless the context clearly dictates otherwise.

As used herein, the term “lower alkyl” denotes a straight- or branched-chain hydrocarbon group containing from 1-8 carbon atoms, for example, methyl, ethyl, propyl, isopropyl, n-butyl, i-butyl, t-butyl and the like. Preferred lower alkyl groups are groups with 1-4 carbon atoms.

The term “lower alkyl substituted by halogen” denotes an alkyl group as defined above, wherein at least one hydrogen atom is replaced by halogen, for example —CF₃, —CHF₂, —CH₂F, —CH₂CF₃, —CF₂CHF₂, —CH₂CH₂CF₃, —CH₂CF₂CF₃ and the like. Preferred lower alkyl substituted by halogen groups are groups having 1-4 carbon atoms.

The term “lower alkoxy” denotes an alkyl residue as defined above, which is attached via an oxygen atom, for example, methoxy, ethoxy, propoxy, isopropoxy, n-butoxy, i-butoxy, 2-butoxy, t-butoxy and the like. Preferred alkoxy groups are groups with 1-4 carbon atoms.

The term “lower alkoxy substituted by halogen” denotes an alkoxy group as defined above wherein at least one hydrogen atom is replaced by halogen. Preferred lower alkoxy substituted by halogen groups are groups having 1-4 carbon atoms.

The term “halogen” denotes chlorine, iodine, fluorine and bromine.

The term “cycloalkyl” denotes a saturated carbon ring containing from 3-7 carbon atoms, for example, cyclopropyl, cyclobutyl, cyclpentyl, cyclohexyl, cycloheptyl, and the like.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salt” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like.

“Therapeutically effective amount” means an amount that is effective to prevent, alleviate or ameliorate symptoms of disease or prolong the survival of the subject being treated.

Preferred are compounds of formula I for the above mentioned use, wherein X is —C(R⁹)═.

Especially preferred compounds of the present invention are those wherein R¹ is morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O)O-lower alkyl, 1,1-dioxoisothiazolidin-2-yl, azepan-1-yl, azetidin-1-yl or is NR′R″, for example the following compounds
N-(3-methoxy-phenyl)-4-(4-methyl-piperidin-1-yl)-3-nitro-benzamide,
N-(3-methoxy-phenyl)-3-nitro-4-propylamino-benzamide,
4-benzylamino-N-(3-methoxy-phenyl)-3-nitro-benzamide,
4-ethylamino-N-(3-methoxy-phenyl)-3-nitro-benzamide,
4-isopropylamino-N-(3-methoxy-phenyl)-3-nitro-benzamide,
4-azetidin-1-yl-N-(3-methoxy-phenyl)-3-nitro-benzamide,
N-(3-methoxy-phenyl)-3-nitro-4-pyrrolidin-1-yl-benzamide,
N-(3-methoxy-phenyl)-3-nitro-4-piperidin-1-yl-benzamide,
N-(3-methoxy-phenyl)-3-nitro-4-phenylamino-benzamide,
N-(3-methoxy-phenyl)-4-pyrrolidin-1-yl-3-trifluoromethyl-benzamide,
4-(2-methoxy-ethylamino)-N-(3-methoxy-phenyl)-3-trifluoromethyl-benzamide,
4-azetidin-1-yl-N-(3-methoxy-phenyl)-3-trifluoromethyl-benzamide,
N-(3-methoxy-phenyl)-4-piperidin-1-yl-3-trifluoromethyl-benzamide,
N-(3-methoxy-phenyl)-4-(4-methyl-piperidin-1-yl)-3-trifluoromethyl-benzamide,
N-(3-methoxy-phenyl)-4-propylamino-3-trifluoromethyl-benzamide,
4-butylamino-N-(3-methoxy-phenyl)-3-trifluoromethyl-benzamide,
4-benzylamino-N-(3-methoxy-phenyl)-3-trifluoromethyl-benzamide,
4-ethylamino-N-(3-methoxy-phenyl)-3-trifluoromethyl-benzamide,
4-isopropylamino-N-(3-methoxy-phenyl)-3-trifluoromethyl-benzamide,
N-(3-methoxy-phenyl)-4-morpholin-4-yl-3-trifluoromethyl-benzamide,
N-(3-ethyl-phenyl)-4-pyrrolidin-1-yl-3-trifluoromethyl-benzamide,
N-(3-ethoxy-phenyl)-4-pyrrolidin-1-yl-3-trifluoromethyl-benzamide,
N-(3-isopropyl-phenyl)-4-pyrrolidin-1-yl-3-trifluoromethyl-benzamide,
N-(3-isopropoxy-phenyl)-4-pyrrolidin-1-yl-3-trifluoromethyl-benzamide,
N-(3-acetyl-phenyl)-4-pyrrolidin-1-yl-3-trifluoromethyl-benzamide,
N-(3-fluoro-phenyl)-4-pyrrolidin-1-yl-3-trifluoromethyl-benzamide,
N-(3-chloro-phenyl)-4-pyrrolidin-1-yl-3-trifluoromethyl-benzamide,
N-(3-bromo-phenyl)-4-pyrrolidin-1-yl-3-trifluoromethyl-benzamide,
4-pyrrolidin-1-yl-N-m-tolyl-3-trifluoromethyl-benzamide,
N-(3-difluoromethoxy-phenyl)-4-pyrrolidin-1-yl-3-trifluoromethyl-benzamide,
4-pyrrolidin-1-yl-N-[3-(1,1,2,2-tetrafluoro-ethoxy)-phenyl]-3-trifluoromethyl-benzamide,
(rac,meso)-4-(3,5-dimethyl-piperidin-1-yl)-N-(3-methoxy-phenyl)-3-trifluoromethyl-benzamide,
4-azepan-1-yl-N-(3-methoxy-phenyl)-3-trifluoromethyl-benzamide,
4-(4-cyano-piperidin-1-yl)-N-(3-methoxy-phenyl)-3-trifluoromethyl-benzamide, and
N-(3-methoxy-phenyl)-3-trifluoromethyl-4-(4-trifluoromethyl-piperidin-1-yl) -benzamide.

Further preferred compounds of the present invention for the above mentioned use are those wherein X is —N═ and R¹ is morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O)O-lower alkyl, 1,1-dioxoisothiazolidin-2-yl, azepan-1-yl, azetidin-1-yl or is NR′R″, for example the following compounds
N-(3-chloro-phenyl)-6-piperazin-1-yl-nicotinamide,
N-(3-chloro-phenyl)-6-(4-methyl-piperazin-1-yl)-nicotinamide,
5-chloro-N-(3-chloro-phenyl)-6-methylamino-nicotinamide,
5-chloro-N-(3-chloro-phenyl)-6-isopropylamino-nicotinamide,
5-chloro-N-(3-chloro-phenyl)-6-(2-methoxy-ethylamino)-nicotinamide,
5-chloro-N-(3-chloro-phenyl)-6-pyrrolidin-1-yl-nicotinamide,
3′-chloro-4-methyl-3,4,5,6-tetrahydro-2H-[1,2′]bipyridinyl-5′-carboxylic acid (3-chloro-phenyl)-amide,
5-chloro-N-(3-chloro-phenyl)-6-ethylamino-nicotinamide,
5-chloro-N-(3-chloro-phenyl)-6-propylamino-nicotinamide,
6-butylamino-5-chloro-N-(3-chloro-phenyl)-nicotinamide,
6-azetidin-1-yl-5-chloro-N-(3-chloro-phenyl)-nicotinamide,
3′-chloro-3,4,5,6-tetrahydro-2H-[1,2′]bipyridinyl-5′-carboxylic acid (3-chloro-phenyl)amide,
5-chloro-N-(3-chloro-phenyl)-6-(4-methyl-piperazin-1-yl)-nicotinamide,
N-(3-methoxy-phenyl)-6-pyrrolidin-1-yl-5-trifluoromethyl-nicotinamide,
6-benzylamino-N-(3-methoxy-phenyl)-5-trifluoromethyl-nicotinamide,
6-isopropylamino-N-(3-methoxy-phenyl)-5-trifluoromethyl-nicotinamide,
4-methyl-3′-trifluoromethyl-3,4,5,6-tetrahydro-2H-[1,2′]bipyridinyl-5′-carboxylic acid (3-methoxy-phenyl)-amide,
5-chloro-N-(3-methoxy-phenyl)-6-pyrrolidin-1-yl-nicotinamide,
3′-chloro-4-methyl-3,4,5,6-tetrahydro-2H-[1,2′]bipyridinyl-5′-carboxylic acid (3-methoxy-phenyl)-amide,
6-butylamino-5-chloro-N-(3-methoxy-phenyl)-nicotinamide, and
5-chloro-N-(3-chloro-phenyl)-6-piperazin-1-yl-nicotinamide.

Preferred are further compounds of formula I for the above mentioned use, wherein X is —C(R⁹)═ and R¹ is halogen, for example the following compounds
4-chloro-N-phenyl-3-trifluoromethyl-benzamide,
4-chloro-N-(3-methoxy-phenyl)-3-nitro-benzamide,
4-bromo-N-(3-methoxy-phenyl)-3-nitro-benzamide,
3-chloro-4-fluoro-N-(3-methoxy-phenyl)-benzamide,
3-bromo-4-fluoro-N-(3-methoxy-phenyl)-benzamide,
4-fluoro-N-(3-methoxy-phenyl)-3-trifluoromethyl-benzamide,
4-fluoro-N-(3-methoxy-phenyl)-3-nitro-benzamide,
3,4-dichloro-N-[3-(2,5-dimethyl-imidazol-1-ylmethyl)-phenyl]-benzamide,
3,4-dichloro-N-phenyl-benzamide,
4-chloro-N-(3-methoxy-phenyl)-3-trifluoromethyl-benzamide,
3,4-dichloro-N-phenyl-benzamide,
3,3′,4-trichlorobenzanilide, and
3,4-dichloro-N-(3-chloro-phenyl)-benzamide.

Preferred are further compounds of formula I for the above mentioned use, wherein X is —C(R⁹)═ and R¹ is nitro, for example the following compounds
3-trifluoromethyl-4-nitro-N-phenyl-benzamide and
4-nitro-N-phenyl-3-trifluoromethyl-benzamide.

Preferred are further compounds of formula I for the above mentioned use, wherein X is —C(R⁹)═ and R¹ is hydrogen, for example the following compound
N-(3,4-dichloro-phenyl)-3-methyl-benzamide.

Further preferred are compounds of formula I for the above mentioned use, wherein X is —N═ and R¹ is halogen, for example the following compounds
5,6-dichloro-N-(3-chloro-phenyl)-nicotinamide,
5,6-dichloro-N-(3-methoxy-phenyl)-nicotinamide, and
6-chloro-N-(3-methoxy-phenyl)-5-trifluoromethyl-nicotinamide.

The invention also provides compounds of formula IA

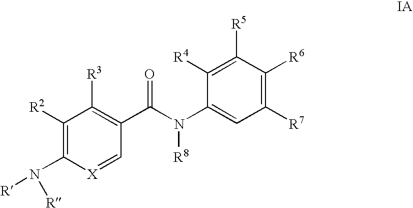

wherein
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR′R″, oxazol-5-yl or halogen; or
R⁵ and R⁶ together with the corresponding C-atoms form a ring comprising —CH═CH—CH═CH—;
R⁸ is hydrogen or lower alkyl;
X is —C(R⁹)═ or —N═;
R⁹ is hydrogen, lower alkoxy, NO₂, or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ—O-lower alkyl;
n is 0, 1, 2 or 3, and
o is 0 or 1,
or a pharmaceutically acceptable acid addition salt thereof;

Specific compounds are for example
N-(3-methoxy-phenyl)-3-nitro-4-propylamino-benzamide,
4-benzylamino-N-(3-methoxy-phenyl)-3-nitro-benzamide,
4-ethylamino-N-(3-methoxy-phenyl)-3-nitro-benzamide,
4-isopropylamino-N-(3-methoxy-phenyl)-3-nitro-benzamide,
N-(3-methoxy-phenyl)-3-nitro-4-phenylamino-benzamide,
4-(2-methoxy-ethylamino)-N-(3-methoxy-phenyl)-3-trifluoromethyl-benzamide,
N-(3-methoxy-phenyl)-4-propylamino-3-trifluoromethyl-benzamide,
4-butylamino-N-(3-methoxy-phenyl)-3-trifluoromethyl-benzamide,
4-benzylamino-N-(3-methoxy-phenyl)-3-trifluoromethyl-benzamide,
4-ethylamino-N-(3-methoxy-phenyl)-3-trifluoromethyl-benzamide,
4-isopropylamino-N-(3-methoxy-phenyl)-3-trifluoromethyl-benzamide,
6-benzylamino-N-(3-methoxy-phenyl)-5-trifluoromethyl-nicotinamide,
6-isopropylamino-N-(3-methoxy-phenyl)-5-trifluoromethyl-nicotinamide, and
6-butylamino-5-chloro-N-(3-methoxy-phenyl)-nicotinamide.

Excluded are the compounds
4-diethylamino-N-phenyl-benzamide,
4-acetylamino-3-nitro-N-phenyl-benzamide,
4-dimethylamino-N-phenyl-benzamide,
which are known from the prior art.

The invention also provides compounds of formula IB

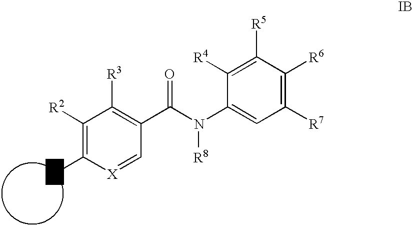

wherein

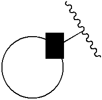

is a cyclic amine group, selected from morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O) O-lower alkyl, 1,1-dioxoisothiazolidin-1-yl, azepan-1-yl and azetidin-1-yl;
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR′R″, oxazol-5-yl or halogen; or
R⁵ and R⁶ together with the corresponding C-atoms form a ring comprising —CH═CH—CH═CH—;
R⁸ is hydrogen or lower alkyl;
X is —C(R⁹)═ or —N═;
R⁹ is hydrogen, lower alkoxy, NO₂, or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ-O-lower alkyl;
n is 0, 1, 2 or 3, and
o is 0 or 1,
or a pharmaceutically acceptable acid addition salt thereof;

Such compounds are for example
N-(3-methoxy-phenyl)-4-(4-methyl-piperidin-1-yl)-3-nitro-benzamide,
4-azetidin-1-yl-N-(3-methoxy-phenyl)-3-nitro-benzamide,
N-(3-methoxy-phenyl)-3-nitro-4-pyrrolidin-1-yl-benzamide,
N-(3-methoxy-phenyl)-3-nitro-4-piperidin-1-yl-benzamide,
N-(3-methoxy-phenyl)-4-pyrrolidin-1-yl-3-trifluoromethyl-benzamide,
4-azetidin-1-yl-N-(3-methoxy-phenyl)-3-trifluoromethyl-benzamide,
N-(3-methoxy-phenyl)-4-piperidin-1-yl-3-trifluoromethyl-benzamide,
N-(3-methoxy-phenyl)-4-(4-methyl-piperidin-1-yl)-3-trifluoromethyl-benzamide,
N-(3-methoxy-phenyl)-4-morpholin-4-yl-3-trifluoromethyl-benzamide,
N-(3-ethyl-phenyl)-4-pyrrolidin-1-yl-3-trifluoromethyl-benzamide,
N-(3-ethoxy-phenyl)-4-pyrrolidin-1-yl-3-trifluoromethyl-benzamide,
N-(3-isopropyl-phenyl)-4-pyrrolidin-1-yl-3-trifluoromethyl-benzamide,
N-(3-isopropoxy-phenyl)-4-pyrrolidin-1-yl-3-trifluoromethyl-benzamide,
N-(3-acetyl-phenyl)-4-pyrrolidin-1-yl-3-trifluoromethyl-benzamide,
N-(3-fluoro-phenyl)-4-pyrrolidin-1-yl-3-trifluoromethyl-benzamide,
N-(3-chloro-phenyl)-4-pyrrolidin-1-yl-3-trifluoromethyl-benzamide,
N-(3-bromo-phenyl)-4-pyrrolidin-1-yl-3-trifluoromethyl-benzamide,
4-pyrrolidin-1-yl-N-m-tolyl-3-trifluoromethyl-benzamide,
N-(3-difluoromethoxy-phenyl)-4-pyrrolidin-1-yl-3-trifluoromethyl-benzamide,
4-pyrrolidin-1-yl-N-[3-(1,1,2,2-tetrafluoro-ethoxy)-phenyl]-3-trifluoromethyl-benzamide,
(rac,meso)-4-(3,5-dimethyl-piperidin-1-yl)-N-(3-methoxy-phenyl)-3-trifluoromethyl-benzamide,
4-azepan-1-yl-N-(3-methoxy-phenyl)-3-trifluoromethyl-benzamide,
4-(4-cyano-piperidin-1-yl)-N-(3-methoxy-phenyl)-3-trifluoromethyl-benzamide,
N-(3-methoxy-phenyl)-3-trifluoromethyl-4-(4-trifluoromethyl-piperidin-1-yl)-benzamide,
4-methyl-3′-trifluoromethyl-3,4,5,6-tetrahydro-2H-[1,2′]bipyridinyl-5′-carboxylic acid (3-methoxy-phenyl)-amide,
5-chloro-N-(3-methoxy-phenyl)-6-pyrrolidin-1-yl-nicotinamide,
3′-chloro-3,4,5,6-tetrahydro-2H-[1,2′]bipyridinyl-5′-carboxylic acid (3-methoxy-phenyl)-amide,
3′-chloro-4-methyl-3,4,5,6-tetrahydro-2H-[1,2′]bipyridinyl-5′-carboxylic acid (3-methoxy-phenyl)-amide, and
5-chloro-N-(3-chloro-phenyl)-6-piperazin-1-yl-nicotinamide.

Some of the compounds of formula I are known compounds and they are either commercially available or can be prepared by methods disclosed in WO 97/03967; WO 99/65449; WO 02/053544; WO 02/059080 or U.S. 2003/0105135 A1;

Scheme I describes a general method for preparing all compounds disclosed in formula I:

The starting materials of formula II are known in the art.

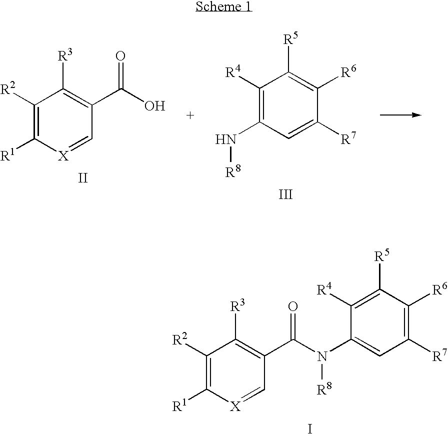

To a solution of N-(3-dimethylaminopropyl)-N′-ethylcarbodiimide hydrochloride (EDC—HCl) in dichloromethane is added a compound of formula III, for example 3-methoxy-aniline and the solution is stirred at ambient temperature for 5 min. To this mixture an acid of formula II, for example 4-fluoro-3-nitrobenzoic acid, is added and the solution is stirred at ambient temperature for about 2 hours.

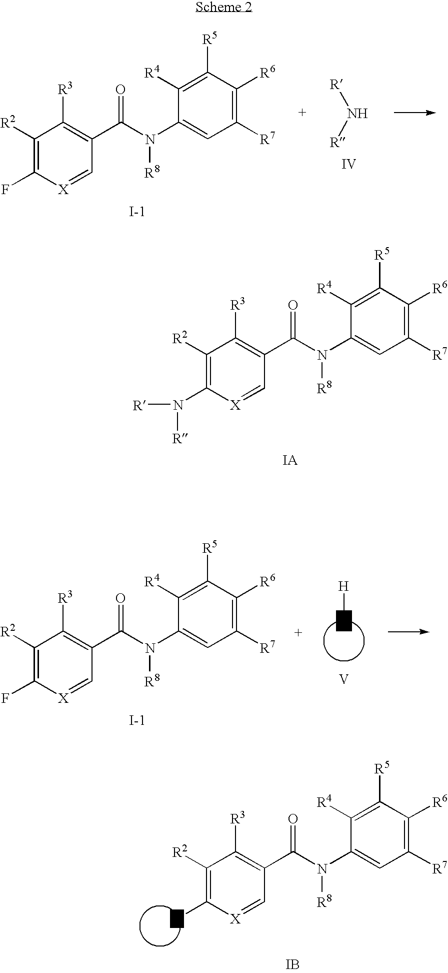

The reaction described in scheme 2 also works with a Cl-substituted compound of formula I-1 instead of a F-substitution.

A solution of a compound of formula I-1 and a compound of formula IV (amine) or V (cyclic amine) in N,N-dimethylformamide or N-methylpyrrolidin-2-one is stirred under microwave irradiation at about 250° C. for 15 minutes. Then the reaction mixture is evaporated and purified to obtain a compound of formula IA or IB.

R′ and R″ in formula IV are independently from each other hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)—O-lower alkyl;

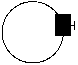

in scheme 2 is a cyclic amine, such as morpholine, pyrrolidine, pyrazole, piperidine, 4-methyl-piperidine, 4-cyano-piperidine, 4-trifluoromethyl-piperidine, piperazine, 4-methyl-piperazine, 3,5-dimethyl-piperidine, piperazine substituted by C(O)O-lower alkyl, 1,1-dioxoisothiazolidine, azepane and azetidine.

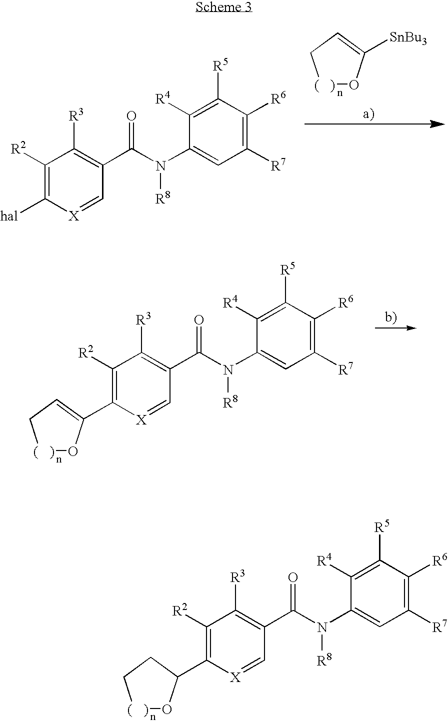

Hal is Cl or Br, n is 1 or 2 and the other definitions are as described above. Following procedures known in the art, stannanes were coupled under Pd catalysis with halo-(het)aryl benzanilides and the resulting vinyl ethers reduced to the saturated ethers.

a) Pd₂dba₃, P(o-furyl)₃, NEt₃, dioxane, rt, 24 h; b) H₂, PtO₂, EtOH, rt, 30 min;

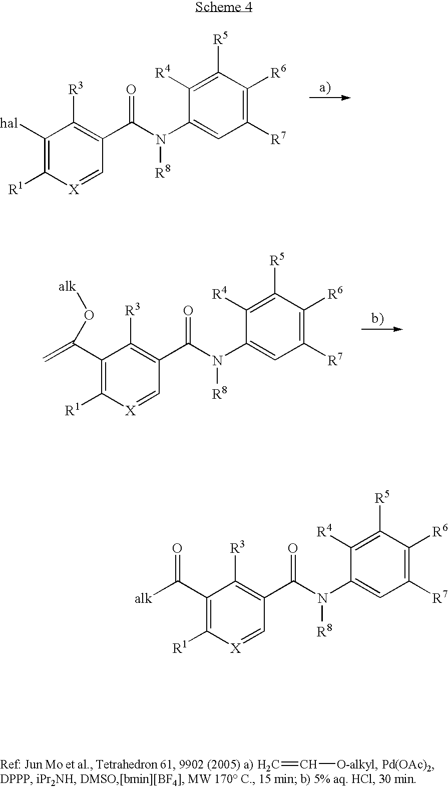

Following procedures known in the art, vinyl ethers were coupled under Pd catalysis with halogenated aryl benzanilides and the resulting vinyl ethers hydrolyzed under acidic conditions to the corresponding ketones.

The preparation of compounds of formula I of the present invention can be carried out in sequential or convergent synthetic routes. Syntheses of the compounds of the invention are shown in the examples. The skills required for carrying out the reaction and purification of the resulting products are known to those skilled in the art. In more detail, the compounds of formula I can be manufactured by the methods given in the examples or by analogous methods. Appropriate reaction conditions for the individual reaction steps are known to a person skilled in the art. The reaction sequence is not limited to the one displayed in the examples, however, depending on the starting materials and their respective reactivity the sequence of reaction steps can be freely altered. Starting materials are either commercially available or can be prepared by methods analogous to the methods described in references cited in the description or in the examples, or by methods known in the art.

The salt formation is effected at room temperature in accordance with methods which are known per se and which are familiar to any person skilled in the art. Not only salts with inorganic acids, but also salts with organic acids come into consideration. Hydrochlorides, hydrobromides, sulphates, nitrates, citrates, acetates, maleates, succinates, methan-sulphonates, p-toluenesulphonates and the like are examples of such salts.

The compounds of formula I and their pharmaceutically usable addition salts possess valuable pharmacological properties. Specifically, it has been found that the compounds of the present invention have a good affinity to the trace amine associated receptors (TAARs), especially TAAR1.

The compounds were investigated in accordance with the test given hereinafter.

### Materials and Methods

### Construction of TAAR Expression Plasmids and Stably Transfected Cell Lines

For the construction of expression plasmids the coding sequences of human, rat and mouse TAAR 1 were amplified from genomic DNA essentially as described by Lindemann et al. [(2005) Genomics 85, 372-385]. The Expand High Fidelity PCR System (Roche Diagnostics) was used with 1.5 mM Mg²⁺ and purified PCR products were cloned into pCR2.1-TOPO cloning vector (Invitrogen) following the instructions of the manufacturer. PCR products were subcloned into the pIRESneo2 vector (BD Clontech, Palo Alto, Calif.), and expression vectors were sequence verified before introduction in cell lines.

HEK293 cells (ATCC #CRL-1573) were cultured essentially as described Lindemann et al. (2005). For the generation of stably transfected cell lines HEK293 cells were transfected with the pIRESneo2 expression plasmids containing the TAAR coding sequences (described above) with Lipofectamine 2000 (Invitrogen) according to the instructions of the manufacturer, and 24 hrs post transfection the culture medium was supplemented with 1 mg/ml G418 (Sigma, Buchs, Switzerland). After a culture period of about 10 d clones were isolated, expanded and tested for responsiveness to trace amines (all compounds purchased from Sigma) with the cAMP Biotrak Enzyme immunoassay (EIA) System (Amersham) following the non-acetylation EIA procedure provided by the manufacturer. Monoclonal cell lines which displayed a stable EC₅₀ for a culture period of 15 passages were used for all subsequent studies.

### Membrane Preparation and Radioligand Binding

Cells at confluence were rinsed with ice-cold phosphate buffered saline without Ca²⁺ and Mg²⁺ containing 10 mM EDTA and pelleted by centrifugation at 1000 rpm for 5 min at 4° C. The pellet was then washed twice with ice-cold phosphate buffered saline and cell pellet was frozen immediately by immersion in liquid nitrogen and stored until use at −80° C. Cell pellet was then suspended in 20 ml HEPES-NaOH (20 mM), pH 7.4 containing 10 mM EDTA, and homogenized with a Polytron (PT 3000, Kinematica) at 10,000 rpm for 10 s. The homogenate was centrifuged at 48,000×g for 30 min at 4° C. and the pellet resuspended in 20 ml HEPES-NaOH (20 mM), pH 7.4 containing 0.1 mM EDTA (buffer A), and homogenized with a Polytron at 10,000 rpm for 10 s. The homogenate was then centrifuged at 48,000×g for 30 min at 4° C. and the pellet resuspended in 20 ml buffer A, and homogenized with a Polytron at 10,000 rpm for 10 s. Protein concentration was determined by the method of Pierce (Rockford, Ill.). The homogenate was then centrifuged at 48,000×g for 10 min at 4° C., resuspended in HEPES-NaOH (20 mM), pH 7.0 including MgCl₂ (10 mM) and CaCl₂ g protein per ml and (2 mM) (buffer B) at 200 homogenized with a Polytron at 10,000 rpm for 10 s.

Binding assay was performed at 4° C. in a final volume of 1 ml, and with an incubation time of 30 min. The radioligand [³H]-rac-2-(1,2,3,4-tetrahydro-1-naphthyl)-2-imidazoline was used at a concentration equal to the calculated K_{d} value of 60 nM to give a bound at around 0.1% of the total added radioligand concentration, and a specific binding which represented approximately 70-80% of the total binding. Non-specific binding was defined as the amount of [³H]-rac-2-(1,2,3,4-tetrahydro-1-naphthyl)-2-imidazoline bound in the presence of the appropriate unlabelled ligand (10 μM). Competing ligands were tested in a wide range of concentrations (10 pM-30 μM). The final dimethylsulphoxide concentration in the assay was 2%, and it did not affect radioligand binding. Each experiment was performed in duplicate. All incubations were terminated by rapid filtration through UniFilter-96 plates (Packard Instrument Company) and glass filter GF/C, pre-soaked for at least 2 h in polyethylenimine 0.3%, and using a Filtermate 96 Cell Harvester (Packard Instrument Company). The tubes and filters were then washed 3 times with 1 ml aliquots of cold buffer B. Filters were not dried and soaked in Ultima gold (45 μl/well, Packard Instrument Company) and bound radioactivity was counted by a TopCount Microplate Scintillation Counter (Packard Instrument Company).

The preferred compounds show a Ki value (μM) in mouse on TAAR1 in the range of 0.002-0.100. Representative compounds are shown in the table below.

TABLE 1

| | Ki value (μM) |
|---|---|
| Example | in mouse |
| 6 | 0.098 |
| 12 | 0.037 |
| 27 | 0.002 |
| 42 | 0.021 |
| 47 | 0.079 |
| 70 | 0.100 |
| 71 | 0.056 |
| 73 | 0.030 |
| 78 | 0.048 |
| 84 | 0.076 |
| 85 | 0.090 |
| 87 | 0.004 |
| 88 | 0.005 |
| 90 | 0.024 |
| 91 | 0.012 |
| 92 | 0.029 |
| 93 | 0.013 |
| 94 | 0.012 |
| 97 | 0.029 |
| 135 | 0.009 |
| 137 | 0.002 |
| 139 | 0.077 |
| 140 | 0.016 |
| 141 | 0.038 |
| 143 | 0.007 |
| 144 | 0.032 |
| 145 | 0.010 |
| 147 | 0.005 |
| 148 | 0.022 |
| 149 | 0.022 |
| 150 | 0.086 |
| 151 | 0.096 |
| 152 | 0.027 |
| 153 | 0.035 |
| 154 | 0.014 |
| 155 | 0.023 |
| 156 | 0.039 |
| 157 | 0.011 |
| 158 | 0.008 |
| 162 | 0.042 |
| 163 | 0.038 |
| 164 | 0.001 |
| 167 | 0.004 |
| 170 | 0.024 |
| 177 | 0.049 |
| 178 | 0.008 |
| 179 | 0.002 |
| 180 | 0.021 |
| 181 | 0.001 |
| 182 | 0.065 |
| 183 | 0.007 |
| 184 | 0.026 |
| 185 | 0.009 |
| 186 | 0.020 |
| 187 | 0.046 |
| 189 | 0.016 |
| 190 | 0.012 |
| 192 | 0.039 |
| 194 | 0.007 |
| 195 | 0.011 |
| 196 | 0.022 |
| 197 | 0.003 |
| 199 | 0.0056 |
| 200 | 0.0597 |
| 201 | 0.0017 |
| 205 | 0.0101 |
| 206 | 0.0207 |
| 207 | 0.0098 |
| 208 | 0.0463 |
| 210 | 0.0348 |
| 216 | 0.0058 |
| 217 | 0.054 |
| 218 | 0.060 |
| 219 | 0.067 |
| 220 | 0.068 |
| 283 | 0.06 |
| 319 | 0.0543 |
| 324 | 0.0674 |

The present invention also provides pharmaceutical compositions containing compounds of the invention, for example, compounds of formula I or pharmaceutically acceptable salts thereof and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The present invention also provides a method for the manufacture of pharmaceutical compositions. Such process comprises bringing one or more compounds of formula I and/or pharmaceutically acceptable acid addition salts thereof and, if desired, one or more other therapeutically valuable substances into a galenical administration form together with one or more therapeutically inert carriers.

The most preferred indications in accordance with the present invention are those, which include disorders of the central nervous system, for example the treatment or prevention of schizophrenia, depression, cognitive impairment and Alzheimer's disease.

The dosage at which compounds of the invention can be administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure

1. Mix items 1, 2, 3 and 4 and granulate with purified water.

2. Dry the granules at 50° C.

3. Pass the granules through suitable milling equipment.

4. Add item 5 and mix for three minutes; compress on a suitable press.

Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure

1. Mix items 1, 2 and 3 in a suitable mixer for 30 minutes.

2. Add items 4 and 5 and mix for 3 minutes.

3. Fill into a suitable capsule.

The following Examples illustrate the present invention without limiting it. All temperatures are given in degrees Celsius.

### Abbreviations

HPLC=high-performance liquid chromatography;

MS=mass spectroscopy.

The following examples are not encompassed by the present claims: 80, 81, 82, 83, 117 and 198.

### EXAMPLE 1

### 4-Methanesulfonyl-N-(3-methoxy-phenyl)-benzamide

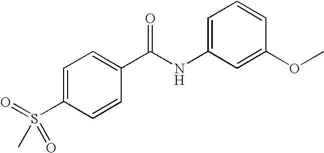

To a solution of 143.8 mg (0.75 mmol) N-(3-dimethylaminopropyl)-N′-ethylcarbodiimide hydrochloride (EDC.HCl) and 91.6 mg (0.75 mmol) 4-dimethylaminopyridine (DMAP) in 2 ml dichloromethane were added 92.3 mg (83.9 μL, 0.75 mmol) 3-methoxy-aniline and the solution stirred at ambient temperature for 5 min. Then this solution was added to 100 mg (0.5 mmol) 4-methanesulfonyl-benzoic acid and the solution stirred at ambient temperature for 18 hours. The reaction mixture was filtered through a cartridge filled with 5 g SCX/silica gel 2:3, pre-washed with 10 ml methanol and 20 ml dichloromethane, and the reaction product eluted with 50 ml dichloromethane. 4-methanesulfonyl-N-(3-methoxy-phenyl)-benzamide was obtained as colourless solid: MS (ISN): 304.4 ((M−H)^{-.}).

In analogy to Example 1 were prepared Examples 2 to 83:

| | | | | | | MS | MS |
|---|---|---|---|---|---|---|---|
| | | | | Chemical | | (ISN): | (ISP): |
| | | | | Name of the | | (M − | (M + |
| Expl. | Structure | RCOOH | R′NHR″ | Product | MW | H)⁻ | H)⁺ |
| 2 | 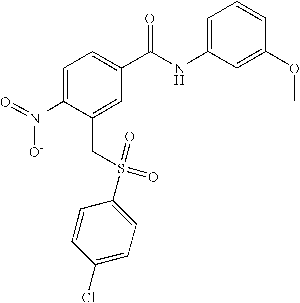 | 3-(4-Chloro-benzenesulfonyl-methyl)-4-nitro-benzoic | 3-Methoxy-aniline | 3-(4-Chloro-benzenesulfonyl-methyl)-N-(3-methoxy-phenyl)-4-nitro-benzamide | 460.89 | 459.1 | |
| 3 | 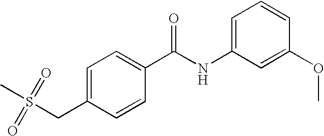 | 4-Methanesulfonyl-methyl-benzoic acid | 3-Methoxy-aniline | 4-Methanesulfonyl-methyl-N-(3-methoxy-phenyl)-benzamide | 319.38 | 318.0 | |
| 4 | 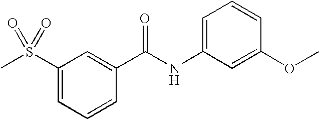 | 3-Methanesulfonyl-benzoic acid | 3-Methoxy-aniline | 3-Methanesulfonyl-N-(3-methoxy-phenyl)-benzamide | 305.35 | 304.2 | |
| 5 | 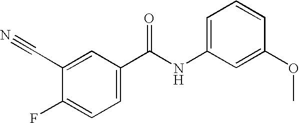 | 3-Cyano-4-fluoro-benzoic acid | 3-Methoxy-aniline | 3-Cyano-4-fluoro-N-(3-methoxy-phenyl)-benzamide | 270.26 | 269.2 | |
| 6 | 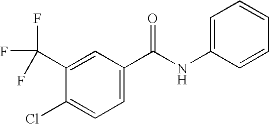 | 4-Chloro-3-trifluoromethyl-benzoic acid | Aniline | 4-Chloro-N-phenyl-3-trifluoromethyl-benzamide | 299.68 | | 300.1and302.2 |
| 7 | 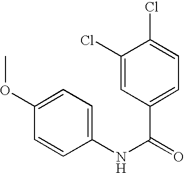 | 3,4-Dichloro-benzoic acid | 4-Methoxy-aniline | 3,4-Dichloro-N-(4-methoxy-phenyl)-benzamide | 296.15 | | 296.0and298.0 |
| 8 | 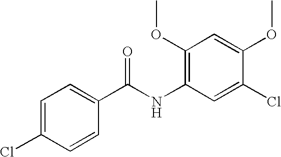 | 4-Chloro-benzoic acid | 5-Chloro-2,4-dimethoxy-aniline | 4-chloro-N-(5-chloro-2,4-dimethoxy-phenyl)-benzamide | 326.18 | | 326.0and328.1 |
| 9 | 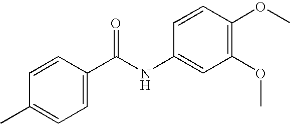 | 4-Methyl-benzoic acid | 3,4-Dimethoxy-aniline | N-(3,4-Dimethoxy-phenyl)-4-methyl-benzamide | 271.32 | 270.4 | |
| 10 | 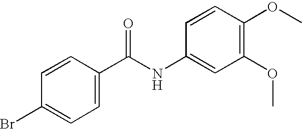 | 4-Bromo-benzoic acid | 3,4-Dimethoxy-aniline | 4-Bromo-N-(3,4-dimethoxy-phenyl)-benzamide | 336.19 | 334.1and336.1 | |
| 11 | 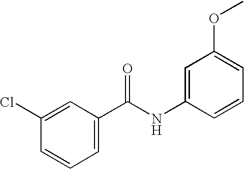 | 3-Chloro-benzoic acid | 3-Methoxy-aniline | 3-Chloro-N-(3-methoxy-phenyl)-benzamide | 261.71 | 260and262.1 | |
| 12 | 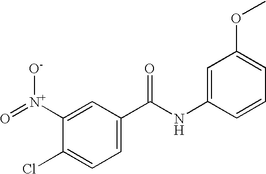 | 4-Chloro-3-nitro-benzoic acid | 3-Methoxy-aniline | 4-Chloro-N-(3-methoxy-phenyl)-3-nitro-benzamide | 306.71 | 305.1 | |
| 13 | 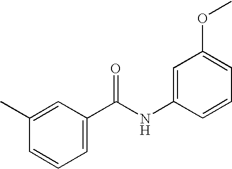 | 3-Methyl-benzoic acid | 3-Methoxy-aniline | N-(3-Methoxy-phenyl)-3-methyl-benzamide | 241.29 | 240.3 | |
| 14 | 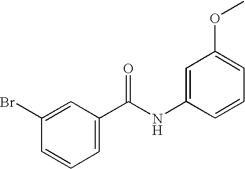 | 3-Bromo-benzoic acid | 3-Methoxy-aniline | 3-Bromo-N-(3-methoxy-phenyl)-benzamide | 306.16 | 304.1and306.2 | |
| 15 | 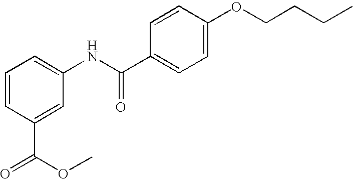 | 4-Butoxy-benzoic acid | 3-Amino-benzoicacidmethylester | 3-(4-Butoxy-benzoylamino)-benzoic acidmethyl ester | 327.38 | 326.3 | |
| 16 | 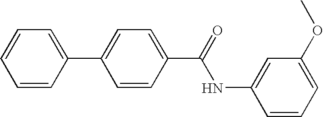 | Biphenyl-4-carboxylicacid | 3-Methoxy-aniline | Biphenyl-4-carboxylic acid(3-methoxy-phenyl)-amide | 303.35 | 302.2 | |
| 17 | 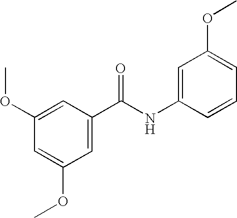 | 3,5-Dimethoxy-benzoic acid | 3-Methoxy-aniline | 3,5-Dimethoxy-N-(3-methoxy-phenyl)-benzamide | 287.31 | 286.1 | |
| 18 | 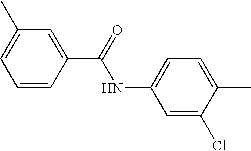 | 3-Methyl-benzoic acid | 3-Chloro-4-methyl-aniline | N-(3-Chloro-4-methyl-phenyl)-3-methyl-benzamide | 259.73 | 257.9and260.0 | |
| 19 | 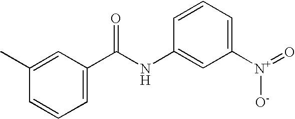 | 3-Methyl-benzoic acid | 3-Nitro-aniline | 3-Methyl-N-(3-nitro-phenyl)-benzamide | 256.26 | 255.1 | |
| 20 | 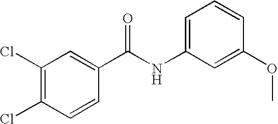 | 3,4-Dichloro-benzoic acid | 3-Methoxy-aniline | 3,4-Dichloro-N-(3-methoxy-phenyl)-benzamide | 296.15 | | 296.0 |
| 21 | 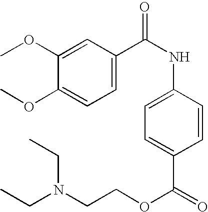 | 3,4-Dimethoxy-benzoic acid | 4-Amino-benzoicacid 2-diethyl-amino-ethylester | 4-(3,4-Dimethoxy-benzoylamino)-benzoic acid2-diethylamino-ethyl ester | 400.48 | | 401.3 |
| 22 | 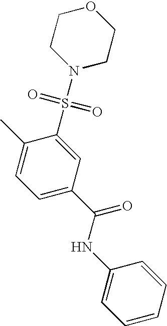 | 4-Methyl-3-(morpholine-4-sulfonyl)-benzoic acid | Aniline | 4-Methyl-3-(morpholine-4-sulfonyl)-N-phenyl-benzamide | 360.43 | | 361.1 |
| 23 | 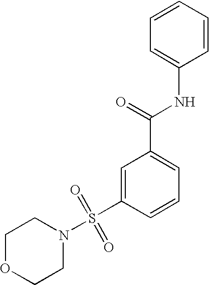 | 3-(Morpholine-4-sulfonyl)-benzoic acid | Aniline | 3-(Morpholine-4-sulfonyl)-N-phenyl-benzamide | 346.4 | 345.2 | |
| 24 | 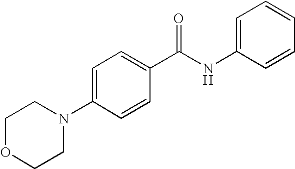 | 4-Morpholin-4-yl-benzoicacid | Aniline | 4-Morpholin-4-yl-N-phenyl-benzamide | 282.34 | | 283.0 |
| 25 | 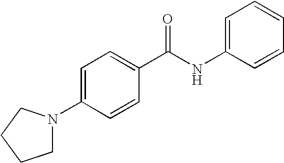 | 4-Pyrrolidin-1-yl-benzoicacid | Aniline | N-Phenyl-4-pyrrolidin-1-yl-benzamide | 266.34 | | 267.0 |
| 26 | 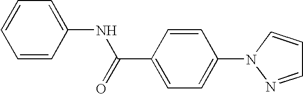 | 4-Pyrazol-1-yl-benzoicacid | Aniline | N-Phenyl-4-pyrazol-1-yl-benzamide | 263.3 | | 263.9 |
| 27 | 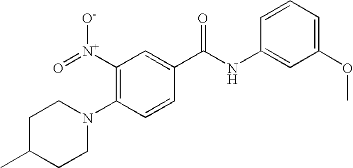 | 4-(4-Methyl-piperidin-1-yl)-3-nitro-benzoic acid | 3-Methoxy-aniline | N-(3-Methoxy-phenyl)-4-(4-methyl-piperidin-1-yl)-3-nitro-benzamide | 369.419 | | 370.1 |
| 28 | 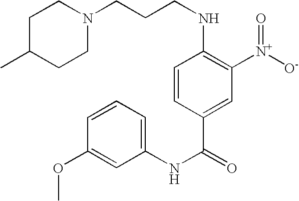 | 4-[3-(4-Methyl-piperidin-1-yl)-propylamino]-3-nitro-benzoic acid | 3-Methoxy-aniline | N-(3-Methoxy-phenyl)-4-[3-(4-methyl-piperidin-1-yl)-propylamino]-3-nitro-benzamide | 426.52 | | 427.3 |
| 29 | 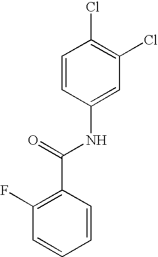 | 2-Fluoro-benzoic acid | 3,4-Dichloro-phenyl-amine | N-(3,4-Dichloro-phenyl)-2-fluoro-benzamide | 284.12 | 282.0and283.1 | |
| 30 | 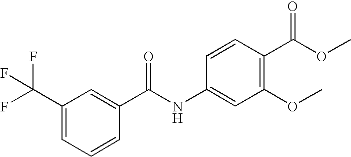 | 3-Trifluoromethyl-benzoic acid | 4-Amino-2-methoxy-benzoicacidmethylester | 2-Methoxy-4-(3-trifluoromethyl-benzoylamino)-benzoic acidmethyl ester | 353.30 | 352.1 | |
| 31 | 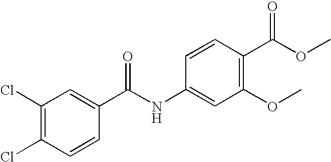 | 3,4-Dichloro-benzoic acid | 4-Amino-2-methoxy-benzoicacidmethylester | 4-(3,4-Dichloro-benzoylamino)-2-methoxy-benzoic acidmethyl ester | 354.19 | 352.0and354.1 | |
| 32 | 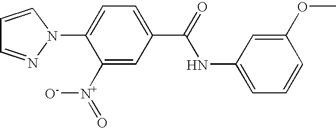 | 3-Nitro-4-pyrazol-1-yl-benzoic acid | 3-Methoxy-aniline | N-(3-Methoxy-phenyl)-3-nitro-4-pyrazol-1-yl-benzamide | 338.33 | 337.2 | |
| 33 | 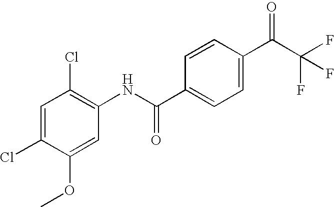 | 4-(2,2,2-Trifluoro-acetyl)-benzoic acid | 2,4-Dichloro-5-methoxy-phenyl-amine | N-(2,4-Dichloro-5-methoxy-phenyl)-4-(2,2,2-trifluoro-acetyl)-benzamide | 392.16 | | 392.1and394.2 |
| 34 | 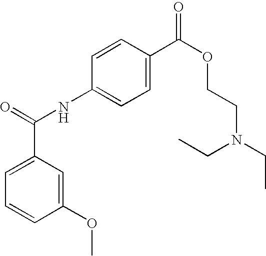 | 3-Methoxy-benzoic acid | 4-Amino-benzoicacid 2-diethyl-amino-ethylester | 4-(3-Methoxy-benzoylamino)-benzoic acid2-diethylamino-ethyl ester | 370.45 | | 371.1 |
| 35 | 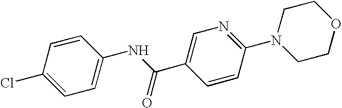 | 6-Morpholin-4-yl-nicotinicacid | 4-Chloro-phenyl-amine | N-(4-Chloro-phenyl)-6-morpholin-4-yl-nicotinamide | 317.77 | | 318.2 |
| 36 | 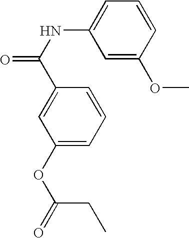 | 3-Propionyloxy-benzoic acid | 3-Methoxy-aniline | Propionic acid3-(3-methoxy-phenyl-carbamoyl)-phenyl ester | 299.33 | | 300.1 |
| 37 | 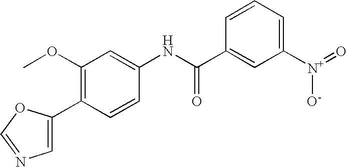 | 3-Nitro-benzoic acid | 3-Methoxy-4-oxazol-5-yl-phenyl-amine | N-(3-Methoxy-4-oxazol-5-yl-phenyl)-3-nitro-benzamide | 339.31 | | 340.0 |
| 38 | 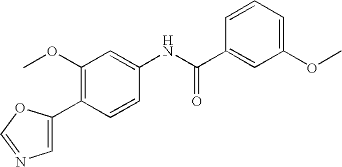 | 3-Methoxy-benzoic acid | 3-Methoxy-4-oxazol-5-yl-phenyl-amine | 3-Methoxy-N-(3-methoxy-4-oxazol-5-yl-phenyl)-benzamide | 324.34 | | 325.3 |
| 39 | 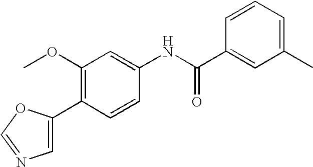 | 3-Methyl-benzoic acid | 3-Methoxy-4-oxazol-5-yl-phenyl-amine | N-(3-Methoxy-4-oxazol-5-yl-phenyl)-3-methyl-benzamide | 308.34 | | 309.3 |
| 40 | 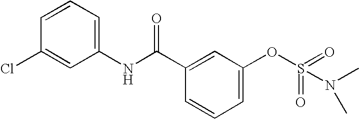 | 3-Dimethyl-sulfamoyloxy-benzoic acid | 3-Chloro-phenyl-amine | Dimethyl-sulfamic acid3-(3-chloro-phenyl-carbamoyl)-phenyl ester | 354.82 | | 355.0and357.1 |
| 41 | 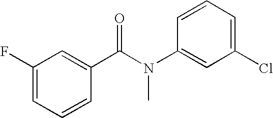 | 3-Fluoro-benzoic acid | (3-Chloro-phenyl)-methyl-amine | N-(3-Chloro-phenyl)-3-fluoro-N-methyl-benzamide | 263.70 | | 263.8and265.9 |
| 42 | 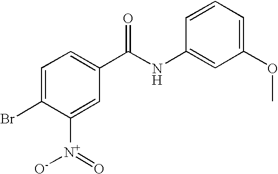 | 4-Bromo-3-nitro-benzoic acid | 3-Methoxy-aniline | 4-Bromo-N-(3-methoxy-phenyl)-3-nitro-benzamide | 351.16 | 349.0and351.1 | |
| 43 | 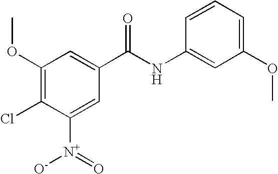 | 4-Chloro-3-methoxy-5-nitro-benzoic acid | 3-Methoxy-aniline | 4-Chloro-3-methoxy-N-(3-methoxy-phenyl)-5-nitro-benzamide | 336.73 | 335.2and337.1 | |
| 44 | 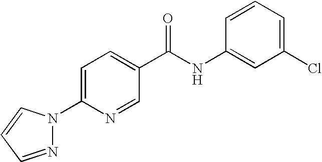 | 6-Pyrazol-1-yl-nicotinicacid | | N-(3-Chloro-phenyl)-6-imidazol-1-yl-nicotinamide | 298.73 | | 299.0 |
| 45 | 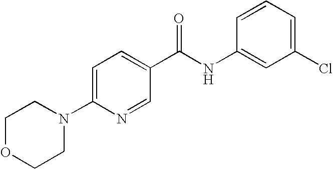 | 6-Morpholin-4-yl-nicotinicacid | 3-Chloro-phenyl-amine | N-(3-Chloro-phenyl)-6-morpholin-4-yl-nicotinamide | 317.77 | | 318.8and320.9 |
| 46 | 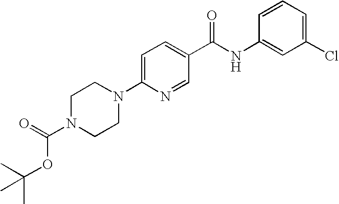 | 4-(5-Carboxy-pyridin-2-yl)-piperazine-1-carboxylicacid tert-butyl ester | 3-Chloro-phenyl-amine | 4-[5-(3-Chloro-phenylcarbamoyl)-pyridin-2-yl]-piperazine-1-carboxylicacid tert-butyl ester | 416.91 | 415.2 | |
| 47 | 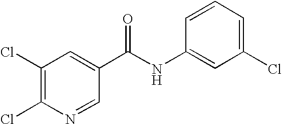 | 5,6-Dichloro-nicotinic acid | 3-Chloro-phenyl-amine | 5,6-Dichloro-N-(3-chloro-phenyl)-nicotinamide | 301.56 | | 301.0and302.9and305.0 |
| 48 | 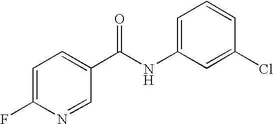 | 6-Fluoro-nicotinic acid | 3-Chloro-phenyl-amine | N-(3-Chloro-phenyl)-6-fluoro-nicotinamide | 250.66 | 249.0and251.1 | |
| 49 | 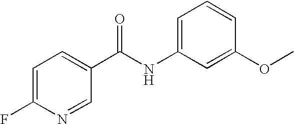 | 6-Fluoro-nicotinic acid | 3-Methoxy-aniline | 6-Fluoro-N-(3-methoxy-phenyl)-nicotinamide | 246.24 | | 247.2 |
| 50 | 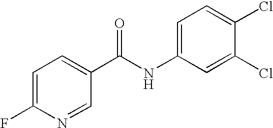 | 6-Fluoro-nicotinic acid | 3,4-Dichloro-phenyl-amine | N-(3,4-Dichloro-phenyl)-6-fluoro-nicotinamide | 285.10 | 282.9and284.0 | |
| 51 | 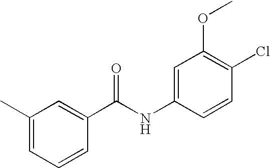 | 3-Methyl-benzoic acid | 4-Chloro-3-methoxy-phenyl-amine | N-(4-Chloro-3-methoxy-phenyl)-3-methyl-benzamide | 275.73 | | 276.0and278.1 |
| 52 | 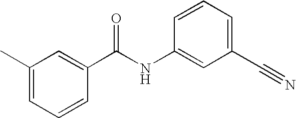 | 2-Methyl-benzoic acid | 3-Amino-benzo-nitrile | N-(3-Cyano-phenyl)-3-methyl-benzamide | 236.27 | | 237.0 |
| 53 | 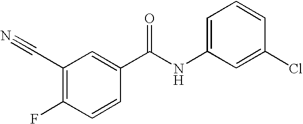 | 3-Cyano-4-fluoro-benzoic acid | 3-Chloro-phenyl-amine | N-(3-Chloro-phenyl)-3-cyano-4-fluoro-benzamide | 274.68 | 273.1and275.2 | |
| 54 | 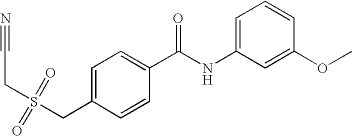 | 4-Cyano-methane-sulfonylmethyl-benzoic acid | 3-Methoxy-aniline | 4-Cyanomethane-sulfonylmethyl-N-(3-methoxy-phenyl)-benzamide | 344.39 | 343.0 | |
| 55 | 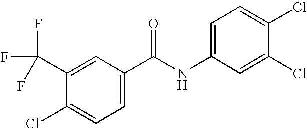 | 4-Chloro-3-trifluoro-methyl-benzoic acid | 3,4-Dichloro-phenyl-amine | 4-Chloro-N-(3,4-dichloro-phenyl)-3-trifluoromethyl-benzamide | 368.57 | 365.9and367.9and371.0 | |
| 56 | 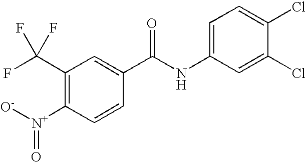 | 4-Nitro-3-trifluoro-methyl-benzoic acid | 3,4-Dichloro-phenyl-amine | N-(3,4-Dichloro-phenyl)-4-nitro-3-trifluoromethyl-benzamide | 379.12 | 377.0and379.0and381.0 | |
| 57 | 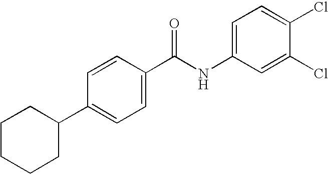 | 4-Cyclohexyl-benzoic acid | 3,4-Dichloro-phenyl-amine | 4-Cyclohexyl-N-(3,4-dichloro-phenyl)-benzamide | 348.27 | 345.9and348.0and350.1 | |
| 58 | 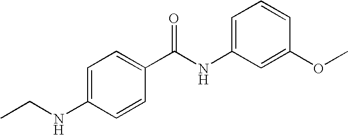 | 4-Ethylamino-benzoic acid | 3-Methoxy-aniline | 4-Ethylamino-N-(3-methoxy-phenyl)-benzamide | 270.33 | 269.1 | |
| 59 | 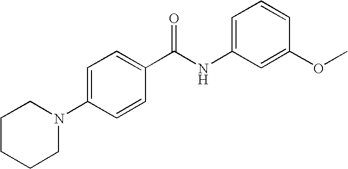 | 4-Piperidin-1-yl-benzoicacid | 3-Methoxy-aniline | N-(3-Methoxy-phenyl)-4-piperidin-1-yl-benzamide | 310.40 | 309.3 | |
| 60 | 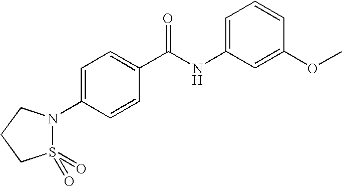 | 4-(1,1-Dioxoiso-thiazolidin-2-yl)benzoicacid | 3-Methoxy-aniline | 4-(1,1-Dioxo-1,l,6-isothiazolidin-2-yl)-N-(3-methoxy-phenyl)-benzamide | 346.40 | 345.0 | |
| 61 | 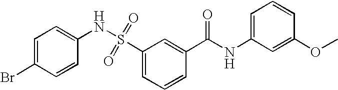 | 3-(4-Bromo-phenyl-sulfamoyl)-benzoic acid | 3-Methoxy-aniline | 3-(4-Bromo-phenyl-sulfamoyl)-N-(3-methoxy-phenyl)-benzamide | 461.34 | 459.0and460.9 | |
| 62 | 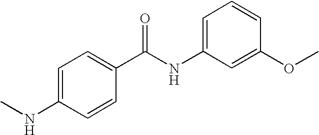 | 4-Methylamino-benzoic acid | 3-Methoxy-aniline | N-(3-Methoxy-phenyl)-4-methylamino-benzamide | 256.30 | | 257.1 |
| 63 | 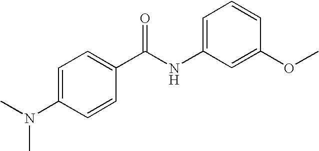 | 4-Dimethyl-amino-benzoicacid | 3-Methoxy-aniline | 4-Dimethyl-amino-N-(3-methoxy-phenyl)-benzamide | 270.33 | | 271.1 |
| 64 | 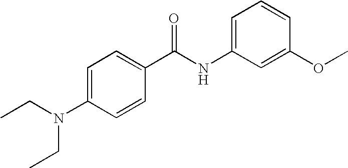 | 4-Diethylamino-benzoic acid | 3-Methoxy-aniline | 4-Diethylamino-N-(3-methoxy-phenyl)-benzamide | 298.39 | | 299.1 |
| 65 | 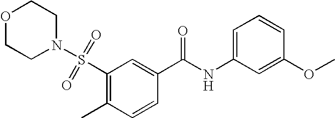 | 4-Methyl-3-(morpholine-4-sulfonyl)-benzoic acid | 3-Methoxy-aniline | N-(3-Methoxy-phenyl)-4-methyl-3-(morpholine-4-sulfonyl)-benzamide | 390.46 | | 391.0 |
| 66 | 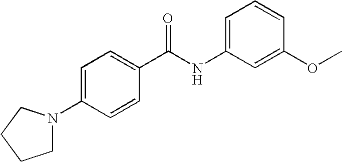 | 4-Pyrrolidin-1-yl-benzoicacid | 3-Methoxy-aniline | N-(3-Methoxy-phenyl)-4-pyrrolidin-1-yl-benzamide | 296.37 | 295.4 | |
| 67 | 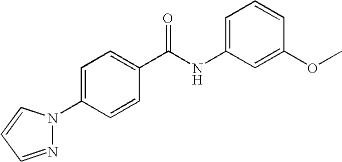 | 4-Pyrazol-1-yl-benzoicacid | 3-Methoxy-aniline | N-(3-Methoxy-phenyl)-4-pyrazol-1-yl-benzamide | 293.33 | 292.1 | |
| 68 | 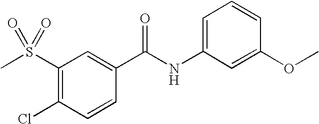 | 4-Chloro-3-methanesulfonyl-benzoic acid | 3-Methoxy-aniline | 4-Chloro-3-methanesulfonyl-N-(3-methoxy-phenyl)-benzamide | 339.80 | 338.0and340.1 | 340.0and342.1 |
| 69 | 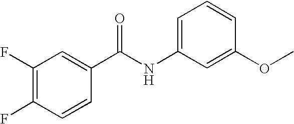 | 3,4-Difluoro-benzoic acid | 3-Methoxy-aniline | 3,4-Difluoro-N-(3-methoxy-phenyl)-benzamide | 263.24 | | 263.8 |
| 70 | 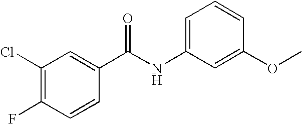 | 3-Chloro-4-fluoro-benzoic acid | 3-Methoxy-aniline | 3-Chloro-4-fluoro-N-(3-methoxy-phenyl)-benzamide | 279.70 | 278.1and280.1 | |
| 71 | 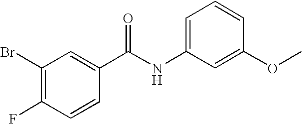 | 3-Bromo-4-fluoro-benzoic acid | 3-Methoxy-aniline | 3-Bromo-4-fluoro-N-(3-methoxy-phenyl)-benzamide | 324.15 | 322.0and324.1 | |
| 72 | 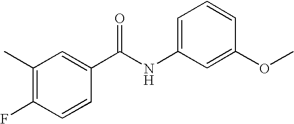 | 4-Fluoro-3-methyl-benzoic acid | 3-Methoxy-aniline | 4-Fluoro-N-(3-methoxy-phenyl)-3-methyl-benzamide | 259.28 | 258.0 | |
| 73 | 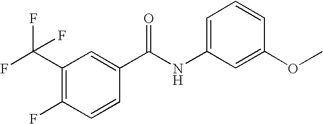 | 4-Fluoro-3-trifluoromethyl-benzoic acid | 3-Methoxy-aniline | 4-Fluoro-N-(3-methoxy-phenyl)-3-trifluoromethyl-benzamide | 313.25 | 312.0 | |
| 74 | 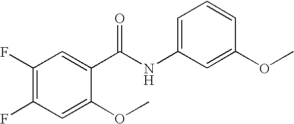 | 4,5-Difluoro-2-methoxy-benzoic acid | 3-Methoxy-aniline | 4,5-Difluoro-2-methoxy-N-(3-methoxy-phenyl)-benzamide | 293.27 | 292.1 | |
| 75 | 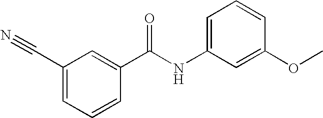 | 3-Cyano-benzoic acid | 3-Methoxy-aniline | 3-Cyanl-N-(3-methoxy-phenyl)-benzamide | 252.27 | 251.2 | |
| 76 | 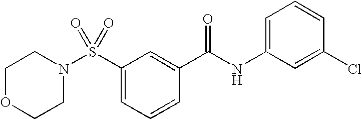 | 3-(Morpholine-4-sulfonyl)-benzoic acid | 3-Chloro-phenyl-amine | N-(3-Chloro-phenyl)-3-(morpholine-4-sulfonyl)-benzamide | 380.85 | 379.0and381.1 | |
| 77 | 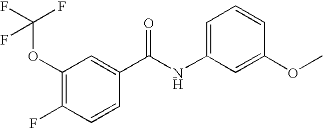 | 4-Fluoro-3-trifluoro-methoxy-benzoic acid | 3-Methoxyaniline | 4-Fluoro-N-(3-methoxy-phenyl)-3-trifluoromethoxy-benzamide | 329.25 | 328.0 | |
| 78 | 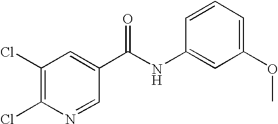 | 5,6-Dichloro-nicotinic acid | 3-Methoxy-aniline | 5,6-Dichloro-N-(3-methoxy-phenyl)-nicotinamide | 297.14 | 295.0and297.1 | |
| 79 | 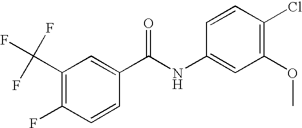 | 4-Fluoro-3-trifluoromethyl-benzoic acid | 4-Chloro-3-methoxy-phenyl-amine | N-(4-Chloro-3-methoxy-phenyl)-4-fluoro-3-trifluoromethyl-benzamide | 347.69 | 346.1and348.2 | |
| 80 | 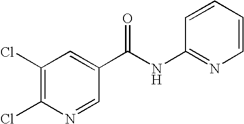 | 5,6-Dichloro-nicotinic acid | Pyridin-2-ylamine | 5,6-Dichloro-N-pyridin-2-yl-nicotinamide | 268.10 | 265.9and268.0 | |
| 81 | 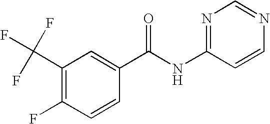 | 4-Fluoro-3-trifluoromethyl-benzoic acid | Pyrimidin-4-ylamine | 4-Fluoro-N-pyrimidin-4-yl-3-trifluoromethyl-benzamide | 285.20 | 284.0 | |
| 82 | 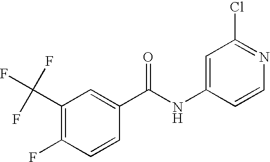 | 4-Fluoro-3-trifluoromethyl-benzoic acid | 2-Chloro-pyridin-4-ylamine | N-(2-Chloro-pyridin-4-yl)-4-fluoro-3-trifluoromethyl-benzamide | 318.66 | 317.0and319.1 | |
| 83 | 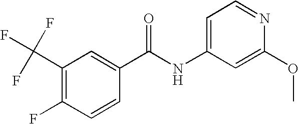 | 4-Fluoro-3-trifluoromethyl-benzoic acid | 2-Methoxy-pyridin-4-ylamine | 4-Fluoro-N-(2-methoxy-pyridin-4-yl)-3-trifluoro-methyl-benzamide | 314.24 | 313.0 | |

### EXAMPLE 84

### N-(3-Chloro-phenyl)-6-piperazin-1-yl-nicotinamide

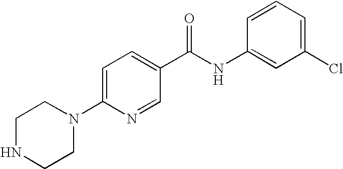

To a solution of 30 mg (0.072 mmol) 4-[5-(3-chloro-phenylcarbamoyl)-pyridin-2-yl]-piperazine-1-carboxylic acid tert-butyl ester (Example 46) in 0.5 ml ethanol were added 1 ml aqueous 1N HCl and the mixture stirred at ambient temperature for 20 hours. Then the mixture was evaporated, the residue taken up in 1N NaOH and extracted three times with tert-butyl methyl ether. The combined organic extracts were washed with brine, dried over Na₂SO₄, filtered and evaporated. N-(3-Chloro-phenyl)-6-piperazin-1-yl-nicotinamide was obtained as an off-white solid: MS (EI): 316.1 and 318.1 (M^{+.}).

### EXAMPLE 85

### 4-Fluoro-N-(3-methoxy-phenyl)-3-nitro-benzamide

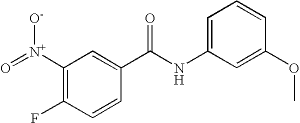

To a solution of 14.38 g (75 mmol) N-(3-dimethylaminopropyl)-N′-ethylcarbodiimide hydrochloride (EDC.HCl) in 150 ml dichloromethane were added 9.26 g (75 mmol) 3-methoxy-aniline and the solution stirred at ambient temperature for 5 min. To this mixture 9.26 g (50 mmol) 4-fluoro-3-nitrobenzoic acid were added and the solution stirred at ambient temperature for 4 hours. Then 150 ml 2N HCl were added, stirred for a few minutes, the organic phase separated and the aqueous phase washed with 50 ml dichloromethane. The two organic extracts were washed successively with 50 ml brine then combined, dried over Na₂SO₄, filtered and evaporated. Re-crystallization of the residue provided 11.11 g 4-fluoro-N-(3-methoxy-phenyl)-3-nitro-benzamide as yellow solid: m.p. 145-146° C.; MS (ISN): 289.0 ((M−H)^{-.}).

### EXAMPLE 86

### N-(3,4-Dichloro-phenyl)-4-fluoro-3-nitro-benzamide

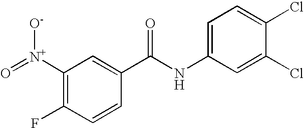

N-(3,4-Dichloro-phenyl)-4-fluoro-3-nitro-benzamide was prepared from 3,4-dichloroaniline and 4-fluoro-3-nitro-benzoic acid in analogy to Example 85: colourless solid: MS (ISN): 327.1, 329.1 and 331.1 ((M−H)^{-.}).

### EXAMPLE 87

### N-(3-Methoxy-phenyl)-3-nitro-4-propylamino-benzamide

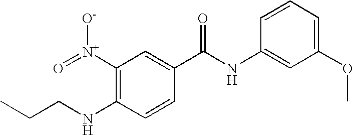

A solution of 35.5 mg (1.1 mmol) propylamine and 145 mg (0.5 mmol) 4-fluoro-N-(3-methoxy-phenyl)-3-nitro-benzamide in 2 ml tetrahydrofuran was stirred at ambient temperature for 70 hours. The reaction mixture was filtered through a cartridge filled with 3 g SCX/silica gel 1:1, pre-washed with 20 ml methanol and 10 ml dichloromethane, and the reaction product eluted with 20 ml dichloromethane. N-(3-methoxy-phenyl)-3-nitro-4-propylamino-benzamide was obtained as orange solid: MS (ISP): 330.1 ((M+H)^{+.}).

In analogy to Example 87 were prepared Examples 88 to 96:

| | | | | Chemical | | MS |
|---|---|---|---|---|---|---|
| | | 4-fluoro- | | Name of the | | (ISP): |
| Expl. | Structure | benzamide | R′NHR″ | Product | MW | (M + H)^{.+} |
| 88 | 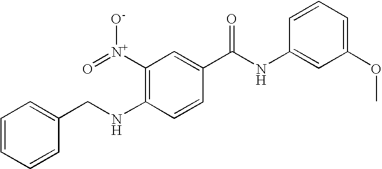 | 4-Fluoro-N-(3-methoxy-phenyl)-3-nitro-benzamide | benzylamine | 4-Benzylamino-N-(3-methoxy-phenyl)-3-nitro-benzamide | 377.40 | 378.1 |
| 89 | 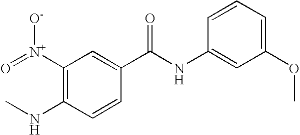 | 4-Fluoro-N-(3-methoxy-phenyl)-3-nitro-benzamide | methylamine | N-(3-Methoxy-phenyl)-4-methylamino-3-nitro-benzamide | 301.30 | 302.0 |
| 90 | 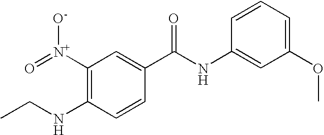 | 4-Fluoro-N-(3-methoxy-phenyl)-3-nitro-benzamide | ethylamine | 4-Ethylamino-N-(3-methoxy-phenyl)-3-nitro-benzamide | 315.33 | 316.0 |
| 91 | 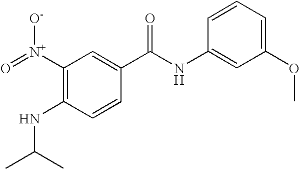 | 4-Fluoro-N-(3-methoxy-phenyl)-3-nitro-benzamide | iso-propylamine | 4-Isopropylamino-N-(3-methoxy-phenyl)-3-nitro-benzamide | 329.35 | 330.2 |
| 92 | 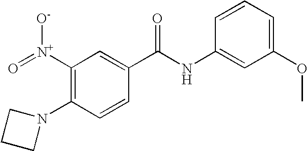 | 4-Fluoro-N-(3-methoxy-phenyl)-3-nitro-benzamide | azetidine | 4-Azetidin-1-yl-N-(3-methoxy-phenyl)-3-nitro-benzamide | 327.34 | 328.0 |
| 93 | 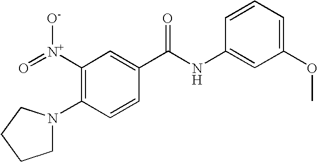 | 4-Fluoro-N-(3-methoxy-phenyl)-3-nitro-benzamide | pyrrolidine | N-(3-Methoxy-phenyl)-3-nitro-4-pyrrolidin-1-yl-benzamide | 341.37 | 342.1 |
| 94 | 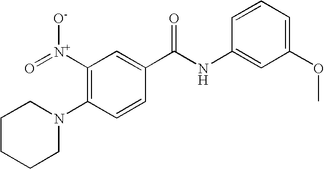 | 4-Fluoro-N-(3-methoxy-phenyl)-3-nitro-benzamide | piperidine | N-(3-Methoxy-phenyl)-3-nitro-4-piperidin-1-yl-benzamide | 355.39 | 356.2 |
| 95 | 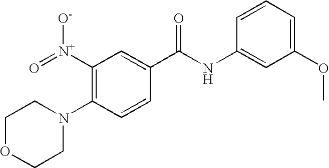 | 4-Fluoro-N-(3-methoxy-phenyl)-3-nitro-benzamide | morpholine | N-(3-Methoxy-phenyl)-4-morpholin-4-yl-3-nitro-benzamide | 357.36 | 358.0 |
| 96 | 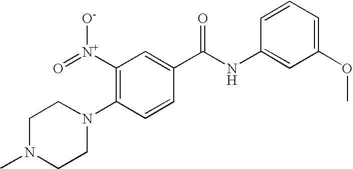 | 4-Fluoro-N-(3-methoxy-phenyl)-3-nitro-benzamide | 1-methylpiprazine | N-(3-Methoxy-phenyl)-4-(4-methyl-piperazin-1-yl)-3-nitro-benzamide | 370.41 | 371.1 |

### EXAMPLE 97

### N-(3-Methoxy-phenyl)-3-nitro-4-phenylamino-benzamide

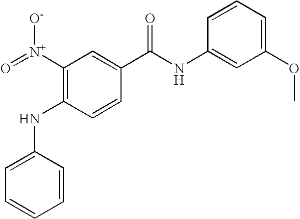

A solution of 102.4 mg (1.1 mmol) aniline and 145 mg (0.5 mmol) 4-fluoro-N-(3-methoxy-phenyl)-3-nitro-benzamide in 2 ml tetrahydrofuran was stirred at 50° C. for 70 hours. The reaction mixture was filtered through a cartridge filled with 4 g SCX/silica gel 1:1, pre-washed with 20 ml methanol and 10 ml dichloromethane, and the reaction product eluted with 20 ml dichloromethane. N-(3-Methoxy-phenyl)-3-nitro-4-phenylamino-benzamide was obtained as orange solid: MS (ISP): 364.0 ((M+H)^{+.}).

### EXAMPLE 98

### 4-Amino-N-(3-methoxy-phenyl)-3-nitro-benzamide

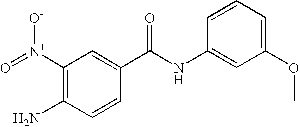

To a solution of 145 mg (0.5 mmol) 4-fluoro-N-(3-methoxy-phenyl)-3-nitro-benzamide in 2 ml N,N-dimethylformamide were added 5 ml of a 25% ammonium hydroxide solution: yellow crystals began to precipitate. After stirring at ambient temperature for 2.5 hours the suspension is diluted with 50 ml tert-butyl methyl ether, the aqueous phase separated and washed twice with tert-butyl methyl ether. The combined organic extracts were washed with brine, dried over Na2SO4, filtered and evaporated. 4-Amino-N-(3-methoxy-phenyl)-3-nitro-benzamide was obtained as yellow solid: MS (ISP): 287.9 ((M+H)^{+.}).

### N-Aryl Nicotinamides

### EXAMPLES 99 TO 124

General Procedure: 1 equivalent nicotinic acid and 1 equivalent (2(1H-7-azabenzotriasol-1-yl)-1,1,3,3-tetramethyl-uronium hexafluoro phosphate (HATU) were dissolved in N,N-dimethylformamide, kept at ambient temperature for 30 minutes and then 1 equivalent N-ethyl-diisopropylamine added. To this solution was added 1 equivalent amine dissolved in N,N-dimethylformamide and the reaction mixture shaken at ambient temperature for 18 hours. The reaction went to completion for all mixtures by heating to 50° C. for additional 20 hours. For purification the reaction mixtures were directly submitted to preparative HPLC.

| | | | | Chemical | | MS | MS |
|---|---|---|---|---|---|---|---|
| Ex- | | 4-fluoro- | | Name of the | | (ISN): | (ISP): |
| ample | Structure | benzamide | R′NHR″ | Product | MW | (M − H)^{.-} | (M + H)^{.+} |
| 99 | 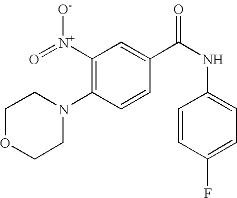 | 4-Morpholin-4-yl-3-nitro-benzoic acid | 4-Fluoro-phenylamine | N-(4-Fluoro-phenyl)-4-morpholin-4-yl-3-nitro-benzamide | 345.33 | 344.2 | |
| 100 | 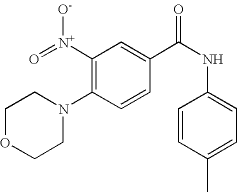 | 4-Morpholin-4-yl-3-nitro-benzoic acid | p-Tolylamine | 4-Morpholin-4-yl-3-nitro-N-p-tolyl-benzamide | 341.37 | | 342.2 |
| 101 | 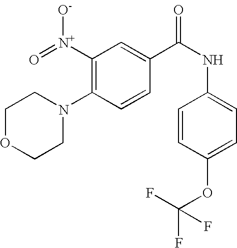 | 4-Morpholin-4-yl-3-nitro-benzoic acid | 4-Trifluoromethoxy-phenylamine | 4-Morpholin-4-yl-3-nitro-N-(4-trifluoromethoxy-phenyl)-benzamide | 411.34 | 410.2 | |
| 102 | 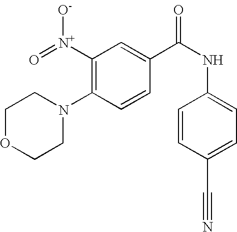 | 4-Morpholin-4-yl-3-nitro-benzoic acid | 4-Amino-benzonitrile | N-(4-Cyano-phenyl)-4-morpholin-4-yl-3-nitro-benzamide | 352.35 | 351.2 | |
| 103 | 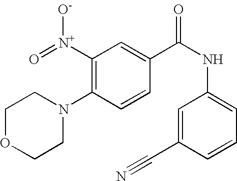 | 4-Morpholin-4-yl-3-nitro-benzoic acid | 3-Amino-benzonitrile | N-(3-Cyano-phenyl)-4-morpholin-4-yl-3-nitro-benzamide | 352.35 | 351.2 | |
| 104 | 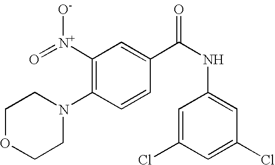 | 4-Morpholin-4-yl-3-nitro-benzoic acid | 3,5-Dichloro-phenylamine | N-(3,5-Dichloro-phenyl)-4-morpholin-4-yl-3-nitro-benzamide | 396.23 | 394.1and396.2 | |
| 105 | 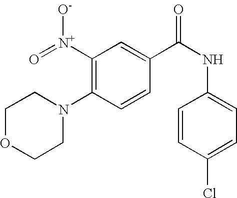 | 4-Morpholin-4-yl-3-nitro-benzoic acid | 4-Chloro-phenylamine | N-(4-Chloro-phenyl)-4-morpholin-4-yl-3-nitro-benzamide | 361.78 | 360.2 | |
| 106 | 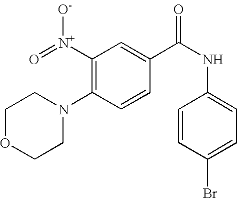 | 4-Morpholin-4-yl-3-nitro-benzoic acid | 4-Bromo-phenylamine | N-(4-Bromo-phenyl)-4-morpholin-4-yl-3-nitro-benzamide | 406.24 | 404.1and406.1 | |
| 107 | 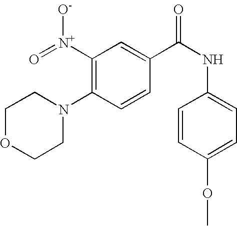 | 4-Morpholin-4-yl-3-nitro-benzoic acid | 4-Methoxy-phenylamine | N-(4-Methoxy-phenyl)-4-morpholin-4-yl-3-nitro-benzamide | 357.37 | | 358.2 |
| 108 | 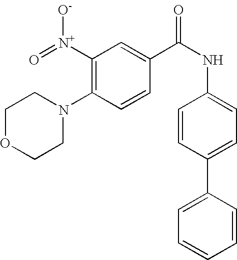 | 4-Morpholin-4-yl-3-nitro-benzoic acid | Biphenyl-4-ylamine | N-Biphenyl-4-yl-4-morpholin-4-yl-3-nitro-benzamide | 403.44 | 402.2 | |
| 109 | 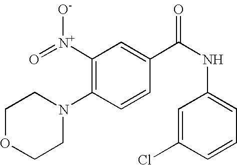 | 4-Morpholin-4-yl-3-nitro-benzoic acid | 3-Chloro-phenylamine | N-(3-Chloro-phenyl)-4-morpholin-4-yl-3-nitro-benzamide | 361.78 | 360.2 | |
| 110 | 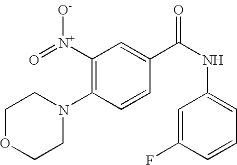 | 4-Morpholin-4-yl-3-nitro-benzoic acid | 3-Fluoro-phenylamine | N-(3-Fluoro-phenyl)-4-morpholin-4-yl-3-nitro-benzamide | 345.33 | 344.2 | |
| 111 | 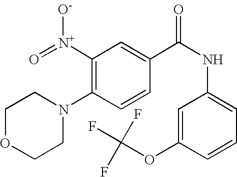 | 4-Morpholin-4-yl-3-nitro-benzoic acid | 3-Trifluoromethoxy-phenylamine | 4-Morpholin-4-yl-3-nitro-N-(3-trifluoromethoxy-phenyl)-benzamide | 411.34 | 410.2 | |
| 112 | 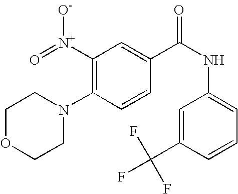 | 4-Morpholin-4-yl-3-nitro-benzoic acid | 3-Trifluoromethyl-phenylamine | 4-Morpholin-4-yl-3-nitro-N-(3-trifluoromethyl-phenyl)-benzamide | 395.34 | 394.2 | |
| 113 | 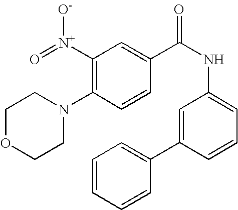 | 4-Morpholin-4-yl-3-nitro-benzoic acid | Biphenyl-3-ylamine | N-Biphenyl-3-yl-4-morpholin-4-yl-3-nitro-benzamide | 403.44 | | 404.3 |
| 114 | 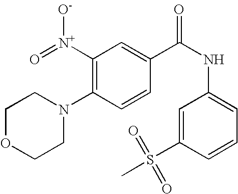 | 4-Morpholin-4-yl-3-nitro-benzoic acid | 3-Methane-sulfonyl-phenylamine | N-(3-Methane-sulfonyl-phenyl)-4-morpholin-4-yl-3-nitro-benzamide | 405.43 | 404.2 | |
| 115 | 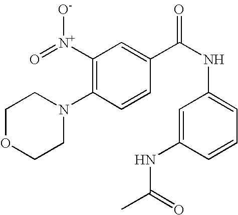 | 4-Morpholin-4-yl-3-nitro-benzoic acid | N-(3-Amino-phenyl)-acetamide | N-(3-Acetylamino-phenyl)-4-morpholin-4-yl-3-nitro-benzamide | 384.39 | | 385.2 |
| 116 | 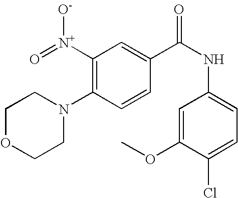 | 4-Morpholin-4-yl-3-nitro-benzoic acid | 4-Chloro-3-methoxy-phenylamine | N-(4-Chloro-3-methoxy-phenyl)-4-morpholin-4-yl-3-nitro-benzamide | 391.81 | 390.2 | |
| 117 | 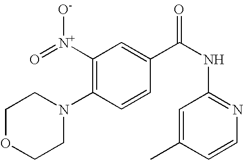 | 4-Morpholin-4-yl-3-nitro-benzoic acid | 4-Methyl-pyridin-2-ylamine | N-(4-Methyl-pyridin-2-yl)-4-morpholin-4-yl-3-nitro-benzamide | 342.35 | | 343.2 |
| 118 | 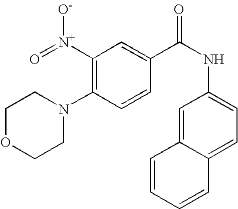 | 4-Morpholin-4-yl-3-nitro-benzoic acid | Naphthalen-2-ylamine | 4-Morpholin-4-yl-N-naphthalen-2-yl-3-nitro-benzamide | 377.40 | 376.3 | |
| 119 | 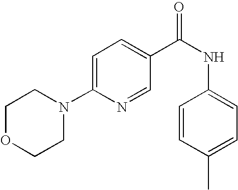 | 6-Morpholin-4-yl-nicotinicacid | p-Tolylamine | 6-Morpholin-4-yl-N-p-tolyl-nicotinamide | 297.36 | | 298.2 |
| 120 | 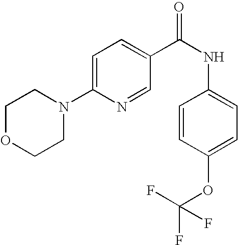 | 6-Morpholin-4-yl-nicotinicacid | 4-Trifluoromethoxy-phenylamine | 6-Morpholin-4-yl-N-(4-trifluoromethoxy-phenyl)-nicotinamide | 367.33 | | 368.2 |
| 121 | 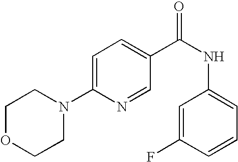 | 6-Morpholin-4-yl-nicotinicacid | 3-Fluoro-phenylamine | N-(3-Fluoro-phenyl)-6-morpholin-4-yl-nicotinamide | 301.32 | 300.2 | |
| 122 | 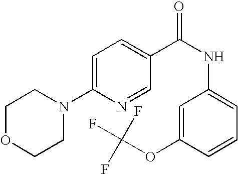 | 6-Morpholin-4-yl-nicotinicacid | 3-Trifluoromethoxy-phenylamine | 6-Morpholin-4-yl-N-(3-trifluoromethoxy-phenyl)-nicotinamide | 367.33 | 366.2 | |
| 123 | 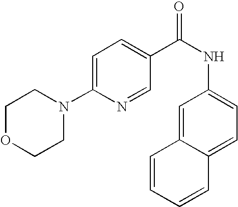 | 6-Morpholin-4-yl-nicotinicacid | Naphthalen-2-ylamine | 6-Morpholin-4-yl-N-naphthalen-2-yl-nicotinamide | 333.39 | | 334.2 |
| 124 | 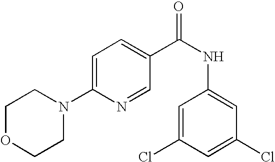 | 6-Morpholin-4-yl-nicotinicacid | 3,5-Dichloro-phenylamine | ′N-(3,5-Dichloro-phenyl)-6-morpholin-4-yl-nicotinamide | 352.22 | | 352.1and354.1 |

### EXAMPLE 125

### 6-Benzylamino-N-(3-chloro-phenyl)-nicotinamide

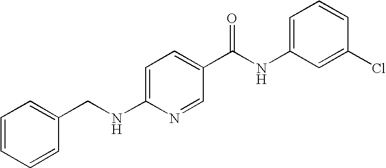

A solution of 80 mg (0.32 mmol) N-(3-chloro-phenyl)-6-fluoro-nicotinamide (Example 48) and 51 mg (0.48 mmol) benzylamine in 1 ml N,N-dimethylformamide was stirred at ambient temperature for 20 hours. Then the reaction mixture was evaporated under reduced pressure and the residue purified by flash-chromatography on silica gel with heptane/ethyl acetate 1:1 as eluent. 6-Benzylamino-N-(3-chloro-phenyl)-nicotinamide was obtained as colourless solid: MS (ISP): 337.9 and 340.0 ((M+H)^{+.}).

In analogy to Example 125 were prepared Examples 126 to 141:

| | | 4-fluoro- | | Chemical | | MS | MS |
|---|---|---|---|---|---|---|---|
| | | nicotin-or | | Name of the | | (ISN): | (ISP): |
| Expl. | Structure | benz-amide | R′NHR″ | Product | MW | (M − H)^{.-} | (M + H)^{.+} |
| 126 | 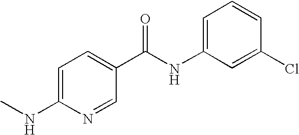 | N-(3-Chloro-phenyl)-6-fluoro-nicotinamide | Methylamine | N-(3-Chloro-phenyl)-6-methylamino-nicotinamide | 261.71 | | 261.9and264.0 |
| 127 | 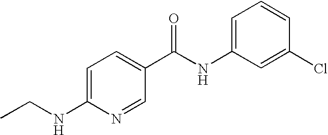 | N-(3-Chloro-phenyl)-6-fluoro-nicotinamide | Ethylamine | N-(3-Chloro-phenyl)-6-ethylamino-nicotinamide | 275.74 | | 276.0and278.1 |
| 128 | 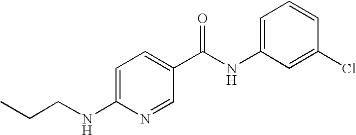 | N-(3-Chloro-phenyl)-6-fluoro-nicotinamide | Propylamine | N-(3-Chloro-phenyl)-6-propylamino-nicotinamide | 289.76 | | 290.0and292.1 |
| 129 | 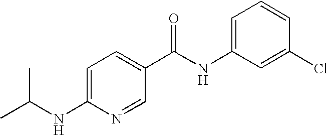 | N-(3-Chloro-phenyl)-6-fluoro-nicotinamide | iso-Propylamine | N-(3-Chloro-phenyl)-6-isopropylamino-nicotinamide | 289.76 | | 290.0and292.1 |
| 130 | 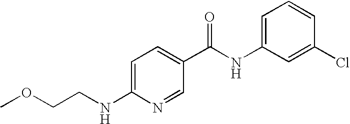 | N-(3-Chloro-phenyl)-6-fluoro-nicotinamide | 2-Methoxy-ethylamine | N-(3-Chloro-phenyl)-6-(2-methoxy-ethylamino)-nicotinamide | 305.76 | | 306.1and308.1 |
| 131 | 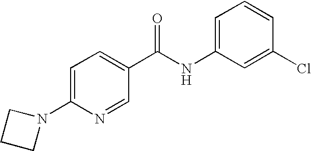 | N-(3-Chloro-phenyl)-6-fluoro-nicotinamide | Azetidine | 6-Azetidin-1-yl-N-(3-chloro-phenyl)-nicotinamide | 287.75 | | 287.9and290.0 |
| 132 | 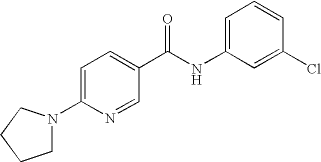 | N-(3-Chloro-phenyl)-6-fluoro-nicotinamide | Pyrrolidine | N-(3-Chloro-phenyl)-6-pyrrolidin-1-yl-nicotinamide | 301.78 | | 302.0and304.1 |
| 133 | 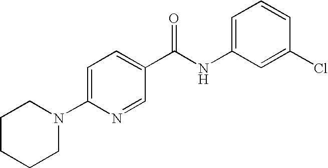 | N-(3-Chloro-phenyl)-6-fluoro-nicotinamide | Piperidine | 3,4,5,6-Tetrahydro-2H-[1,2′]bipyridinyl-5′-carboxylicacid (3-chloro-phenyl)-amide | 315.80 | | 316.0and318.1 |
| 134 | 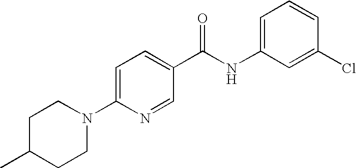 | N-(3-Chloro-phenyl)-6-fluoro-nicotinamide | 4-Methyl-piperidine | 4-Methyl-3,4,5,6-tetrahydro-2H-[1,2′]bipyridinyl-5′-carboxylicacid (3-chloro-phenyl)-amide | 329.83 | 328.1and330.1 | |
| 135 | 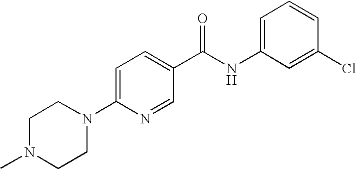 | N-(3-Chloro-phenyl)-6-fluoro-nicotinamide | 1-Methyl-piprazine | N-(3-Chloro-phenyl)-6-(4-methyl-piperazin-1-yl)-nicotinamide | 330.82 | | 331.1and333.2 |
| 136 | 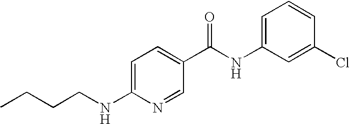 | N-(3-Chloro-phenyl)-6-fluoro-nicotinamide | Butylamine | 6-Butylamino-N-(3-chloro-phenyl)-nicotinamide | 303.79 | | 304.0and306.1 |
| 137 | 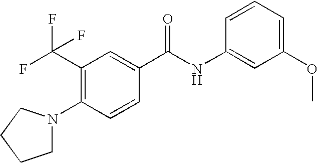 | 4-Fluoro-N-(3-methoxy-phenyl)-3-trifluoro-methyl-benzamide | Pyrrolidine | N-(3-Methoxy-phenyl)-4-pyrrolidin-1-yl-3-trifluoromethyl-benzamide | 364.37 | | 365.0 |
| 138 | 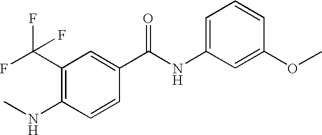 | 4-Fluoro-N-(3-methoxy-phenyl)-3-trifluoro-methyl-benzamide | Methylamine | N-(3-Methoxy-phenyl)-4-methylamino-3-trifluoromethyl-benzamide | 324.30 | | 325.3 |
| 139 | 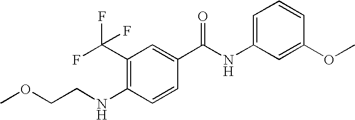 | 4-Fluoro-N-(3-methoxy-phenyl)-3-trifluoro-methyl-benzamide | 2-Methoxy-ethylamine | 4-(2-Methoxy-ethylamino)-N-(3-methoxy-phenyl)-3-trifluoromethyl-benzamide | 368.35 | | 369.1 |
| 140 | 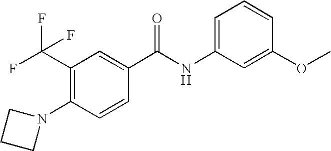 | 4-Fluoro-N-(3-methoxy-phenyl)-3-trifluoro-methyl-benzamide | Azetidine | 4-Azetidin-1-yl-N-(3-methoxy-phenyl)-3-trifluoromethyl-benzamide | 350.34 | | 351.1 |
| 141 | 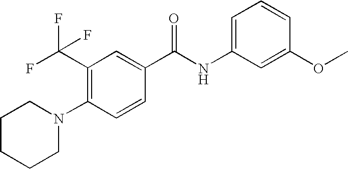 | 4-Fluoro-N-(3-methoxy-phenyl)-3-trifluoro-methyl-benzamide | Piperidine | N-(3-Methoxy-phenyl)-4-piperidin-1-yl-3-trifluoromethyl-benzamide | 378.39 | | 379.2 |

### EXAMPLE 142

### N-(3-Chloro-phenyl)-6-dimethylamino-nicotinamide

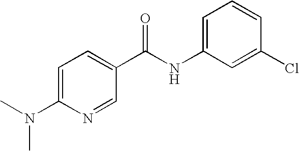

A solution of 80 mg (0.32 mmol) N-(3-chloro-phenyl)-6-fluoro-nicotinamide (Example 48) in 1 ml N,N-dimethylformamide was stirred under microwave irradiation at 200° C. for 45 minutes. Then the reaction mixture was evaporated under reduced pressure and the residue purified by flash-chromatography on silica gel with heptane/ethyl acetate 1:1 as eluent. N-(3-chloro-phenyl)-6-dimethylamino-nicotinamide was obtained as colourless solid: MS (ISP): 276.0 and 278.1 ((M+H)^{+.}).

### EXAMPLE 143

### N-(3-Methoxy-phenyl)-4-(4-methyl-piperidin-1-yl)-3-trifluoromethyl-benzamide

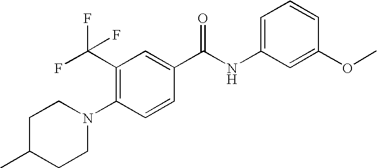

A solution of 100 mg (0.32 mmol) 4-fluoro-N-(3-methoxy-phenyl)-3-trifluoromethyl-benzamide (Example 73) and 57 mg (0.97 mmo;) propylamine in 1 ml 1-methyl-2-pyrrolidinone was stirred under microwave irradiation at 250° C. for 15 minutes. Then the reaction mixture was evaporated under reduced pressure and the residue purified by flash-chromatography on silica gel with a heptane/ethyl acetate gradient with 10% to 20% ethyl acetate as eluent. N-(3-Methoxy-phenyl)-4-(4-methyl-piperidin-1-yl)-3-trifluoromethyl-benzamide was obtained as colorless solid: MS (ISP): 392.9 ((M+H)^{+.}).

In analogy to Example 143 were prepared Examples 144 to 165:

| | | | | Chemical | Rxn | Rxn | MS | MS |
|---|---|---|---|---|---|---|---|---|
| | | 4-fluoro- | | Name of the | Temp. | time | (ISN): | (ISP): |
| Expl. | Structure | benzamide | R′NHR″ | Product | (° C.) | min. | (M − H)^{.-} | (M + H)^{.+} |
| 144 | 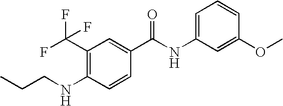 | 4-Fluoro-N-(3-methoxy-phenyl)-3-trifluoromethyl-benzamide | Propylamine | N-(3-Methoxy-phenyl)-4-propylamino-3-trifluoromethyl-benzamide | 200 | 15 | | 353.1 |
| 145 | 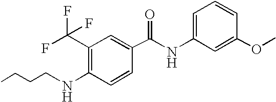 | 4-Fluoro-N-(3-methoxy-phenyl)-3-trifluoromethyl-benzamide | Butylamine | 4-Butylamino-N-(3-methoxy-phenyl)-3-trifluoromethyl-benzamide | 200 | 15 | | 367.0 |
| 146 | 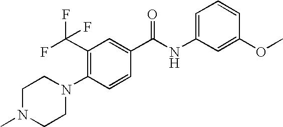 | 4-Fluoro-N-(3-methoxy-phenyl)-3-trifluoromethyl-benzamide | 1-Methyl-piprazine | N-(3-Methoxy-phenyl)-4-(4-methyl-piperazin-1-yl)-3-trifluoromethyl-benzamide | 200 | 15 | | 394.0 |
| 147 | 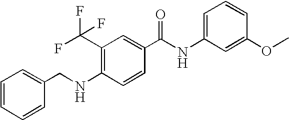 | 4-Fluoro-N-(3-methoxy-phenyl)-3-trifluoromethyl-benzamide | Benzylamine | 4-Benzylamino-N-(3-methoxy-phenyl)-3-trifluoromethyl-benzamide | 250 | 15 | | 401.2 |
| 148 | 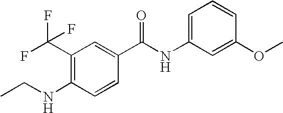 | 4-Fluoro-N-(3-methoxy-phenyl)-3-trifluoromethyl-benzamide | Ethylamine | 4-Ethylamino-N-(3-methoxy-phenyl)-3-trifluoromethyl-benzamide | 250 | 15 | 337.1 | |
| 149 | 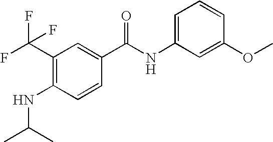 | 4-Fluoro-N-(3-methoxy-phenyl)-3-trifluoromethyl-benzamide | iso-Propylamine | 4-Isopropylamino-N-(3-methoxy-phenyl)-3-trifluoromethyl-benzamide | 250 | 15 | 351.3 | |
| 150 | 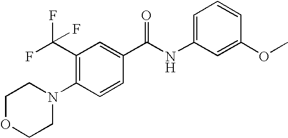 | 4-Fluoro-N-(3-methoxy-phenyl)-3-trifluoromethyl-benzamide | Morpholine | N-(3-Methoxy-phenyl)-4-morpholin-4-yl-3-trifluoromethyl-benzamide | 250 | 15 | | 381.2 |
| 151 | 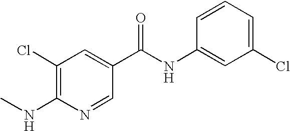 | 5,6-Dichloro-N-(3-chloro-phenyl)-nicotinamide | Methylamine | 5-Chloro-N-(3-chloro-phenyl)-6-methylamino-nicotinamide | 120 | 15 | 294.0and296.1 | |
| 152 | 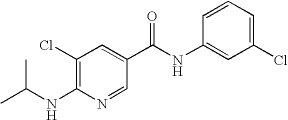 | 5,6-Dichloro-N-(3-chloro-phenyl)-nicotinamide | iso-Propylamine | 5-Chloro-N-(3-chloro-phenyl)-6-isopropylamino-nicotinamide | 120 | 15 | | 324.1and326.0 |
| 153 | 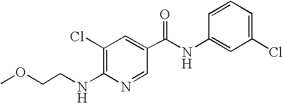 | 5,6-Dichloro-N-(3-chloro-phenyl)-nicotinamide | 2-Methoxy-ethylamine | 5-Chloro-N-(3-chloro-phenyl)-6-(2-methoxy-ethylamino)-nicotinamide | 120 | 15 | | 339.9and342.0and344.1 |
| 154 | 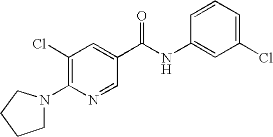 | 5,6-Dichloro-N-(3-chloro-phenyl)-nicotinamide | Pyrrolidine | 5-Chloro-N-(3-chloro-phenyl)-6-pyrrolidin-1-yl-nicotinamide | 120 | 15 | | 336.0and338.0and340.0 |
| 155 | 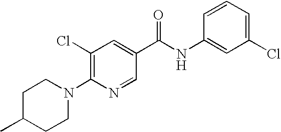 | 5,6-Dichloro-N-(3-chloro-phenyl)-nicotinamide | 4-Methyl-piperidine | 3′-Chloro-4-methyl-3,4,5,6-tetrahydro-2H-[1,2′]bipyridinyl-5′-carboxylicacid (3-chloro-phenyl)-amide | 250 | 15 | | 363.9and366.0 |
| 156 | 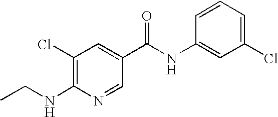 | 5,6-Dichloro-N-(3-chloro-phenyl)-nicotinamide | Ethylamine | 5-Chloro-N-(3-chloro-phenyl)-6-ethylamino-nicotinamide | 120 | 15 | | 309.9and312.0 |
| 157 | 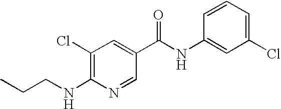 | 5,6-Dichloro-N-(3-chloro-phenyl)-nicotinamide | Propylamine | 5-Chloro-N-(3-chloro-phenyl)-6-propylamino-nicotinamide | 120 | 15 | | 324.0and326.1 |
| 158 | 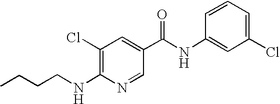 | 5,6-Dichloro-N-(3-chloro-phenyl)-nicotinamide | Butylamine | 6-Butylamino-5-chloro-N-(3-chloro-phenyl)-nicotinamide | 120 | 15 | | 337.9and339.9 |
| 159 | 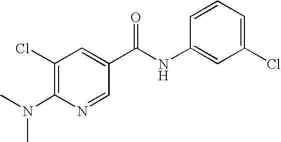 | 5,6-Dichloro-N-(3-chloro-phenyl)-nicotinamide | Dimethylamine | 5-Chloro-N-(3-chloro-phenyl)-6-dimethylamino-nicotinamide | 120 | 15 | | 309.9and312.0 |
| 160 | 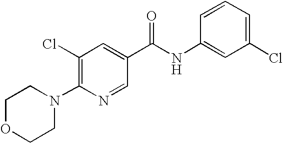 | 5,6-Dichloro-N-(3-chloro-phenyl)-nicotinamide | Morpholine | 5-Chloro-N-(3-chloro-phenyl)-6-morpholin-4-yl-nicotinamide | 120 | 15 | | 352.0and354.1 |
| 161 | 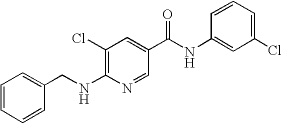 | 5,6-Dichloro-N-(3-chloro-phenyl)-nicotinamide | Benzylamine | 6-Benzylamino-5-chloro-N-(3-chloro-phenyl)-nicotinamide | 120 | 15 | 370.0and372.0 | |
| 162 | 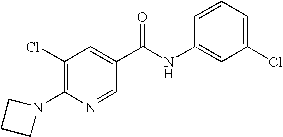 | 5,6-Dichloro-N-(3-chloro-phenyl)-nicotinamide | Azetidine | 6-Azetidin-1-yl-5-chloro-N-(3-chloro-phenyl)-nicotinamide | 120 | 15 | | 322.1and324.1 |
| 163 | 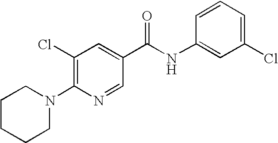 | 5,6-Dichloro-N-(3-chloro-phenyl)-nicotinamide | Piperidine | 3′-Chloro-3,4,5,6-tetrahydro-2H-[1,2′]bipyridinyl-5′-carboxylicacid (3-chloro-phenyl)-amide | 180 | 15 | | 350.1and352.1 |
| 164 | 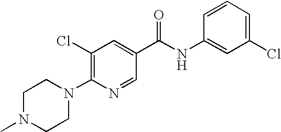 | 5,6-Dichloro-N-(3-chloro-phenyl)-nicotinamide | 1-Methyl-piprazine | 5-Chloro-N-(3-chloro-phenyl)-6-(4-methyl-piperazin-1-yl)-nicotinamide | 120 | 15 | | 364.9and367.0 |
| 165 | 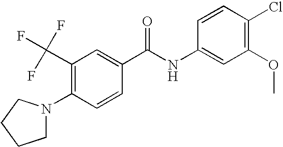 | N-(4-Chloro-3-methoxy-phenyl)-4-fluoro-3-trifluoromethyl-benzamide | Pyrrolidine | N-(4-Chloro-3-methoxy-phenyl)-4-pyrrolidin-1-yl-3-trifluoromethyl-benzamide | 150 | 15 | | 399.0and401.1 |

### EXAMPLE 166

### N-(3-Methoxy-phenyl)-3-(piperazine-1-sulfonyl)-benzamide

### a) 4-(3-Carboxy-benzenesulfonyl)-piperazine-1-carboxylic acid tert-butyl ester

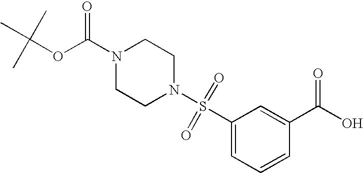

To a cooled solution of 220 mg (1 mmol) of 3-chlorosulfonyl-benzoic acid in 1 ml acetonitrile were added 745 mg (4 mmol) piperazine-1-carboxylic acid tert-butyl ester and 304 mg (3 mmol) triethylamine and then stirred at ambient temperature for 60 hours. The reaction mixture is concentrated under reduced pressure, the residue taken up in 2N NaOH and extracted with ethyl acetate. The aqueous phase is set to pH 1 with concentrated hydrochloric acid and extracted three times with ethyl acetate. The combined organic extracts were washed with brine, dried over Na₂SO₄, filtered and evaporated. 4-(3-Carboxy-benzenesulfonyl)-piperazine-1-carboxylic acid tert-butyl ester was obtained as solid: MS (ISN): 368.8 ((M−H)^{-.}).

### b) 4-[3-(3-Methoxy-phenylcarbamoyl)-benzenesulfonyl]-piperazine-1-carboxylic acid tert-butyl ester

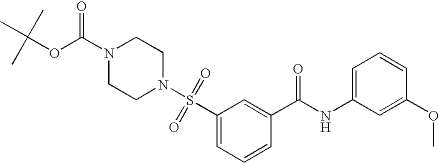

4-[3-(3-Methoxy-phenylcarbamoyl)-benzenesulfonyl]-piperazine-1-carboxylic acid tert-butyl ester was prepared in analogy to Example 1 from 4-(3-carboxy-benzenesulfonyl)-piperazine-1-carboxylic acid tert-butyl ester and 3-methoxy-aniline: colorless solid, MS (ISP): 476.0 ((M+H)^{+.}), 420.1 ((((M+H)-tBu))^{+.}) 98%), 376.3 ((((M+H)-Boc))^{+.}) 100%).

### c) N-(3-Methoxy-phenyl)-3-(piperazine-1-sulfonyl)-benzamide

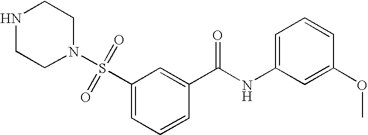

A solution of 110 mg (0.23 mmol) 4-[3-(3-methoxy-phenylcarbamoyl)-benzenesulfonyl]-piperazine-1-carboxylic acid tert-butyl ester in 1 ml ethanol and 10 ml 2N HCl was stirred at 50° C. for 30 min and then concentrated under reduced pressure. The residue was taken up in 2N NaOH and extracted with ethyl acetate. The combined organic extracts were washed with brine, dried over Na₂SO₄, filtered and evaporated. The residue was purified by flash-chromatography on silica gel with dichloromethane/methanol 9:1 as eluent. N-(3-methoxy-phenyl)-3-(piperazine-1-sulfonyl)-benzamide was obtained as colourless solid: MS (ISP): 376.3 ((M+H)^{+.}).

### EXAMPLE 167

### (rac,meso)-3-(3,5-Dimethyl-piperidine-1-sulfonyl)-4-fluoro-N-(3-methoxy-phenyl)-benzamide

### a) (rac,meso)-3-(3,5-Dimethyl-piperidine-1-sulfonyl)-4-fluoro-benzoic acid

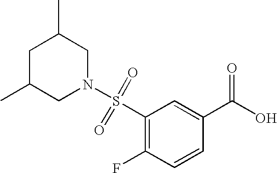

(rac,meso)-3-(3,5-Dimethyl-piperidine-1-sulfonyl)-4-fluoro-benzoic acid was prepared in analogy to Example 166 a) from 3-Chlorosulfonyl-4-fluoro-benzoic acid and racemic (cis,trans-3,5-dimethylpiperidine: colorless solid, MS (ISN): 314.1 ((M−H)^{-.}).

### b) (rac,meso)-3-(3,5-Dimethyl-piperidine-1-sulfonyl)-4-fluoro-N-(3-methoxy-phenyl)-benzamide

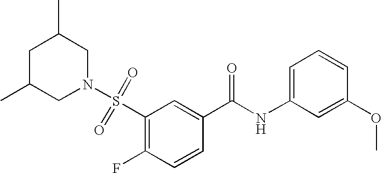

(rac,meso)-3-(3,5-Dimethyl-piperidine-1-sulfonyl)-4-fluoro-N-(3-methoxy-phenyl)-benzamide was prepared in analogy to Example 1 from (rac,meso)-3-(3,5-dimethyl-piperidine-1-sulfonyl)-4-fluoro-benzoic acid and 3-methoxy-aniline: colorless solid, MS (ISP): 421.0 ((M+H)^{+.}).

In analogy to Example 167 were prepared from benzoic acid derivatives known in the literature or commercially available Examples 168 to 176:

| | | | | Chemical | | MS | MS |
|---|---|---|---|---|---|---|---|
| | | | | Name of the | | (ISN): | (ISP): |
| Expl. | Structure | RCOOH | R′NHR″ | Product | MW | (M − H)^{.-} | (M + H)^{.+} |
| 168 | 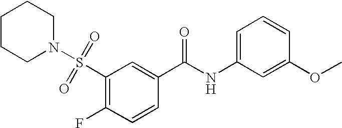 | 4-Fluoro-3-(piperidine-1-sulfonyl)-benzoic acid | 3-Methoxy-aniline | 4-Fluoro-N-(3-methoxy-phenyl)-3-(piperidine-1-sulfonyl)-benzamide | 392.45 | | 393.0 |
| 169 | 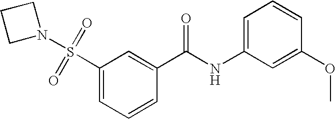 | 3-(Azetidine-1-sulfonyl)-benzoic acid | 3-Methoxy-aniline | 3-(Azetidine-1-sulfonyl)-N-(3-methoxy-phenyl)-benzamide | 346.41 | | 347.1 |
| 170 | 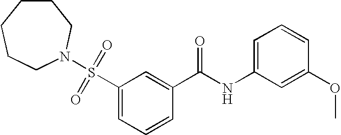 | 3-(Azepane-1-sulfonyl)-benzoic acid | 3-Methoxy-aniline | 3-(Azepane-1-sulfonyl)-N-(3-methoxy-phenyl)-benzamide | 388.49 | | 389.1 |
| 171 | 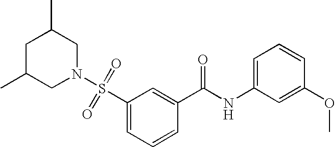 | (rac,meso)-3-(3,5-Dimethyl-piperidine-1-sulfonyl)-benzoic acid | 3-Methoxy-aniline | 3-(3,5-Dimethyl-piperidine-1-sulfonyl)-N-(3-methoxy-phenyl)-benzamide | 402.51 | | 403.2 |
| 172 | 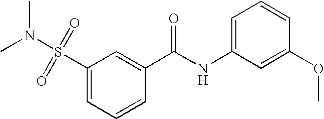 | 3-Dimethyl-sulfamoyl-benzoic acid | 3-Methoxy-aniline | 3-Dimethyl-sulfamoyl-N-(3-methoxy-phenyl)-benzamide | 334.39 | | 335.0 |
| 173 | 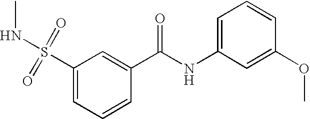 | 3-Methylsulfamoyl-benzoic acid | 3-Methoxy-aniline | N-(3-Methoxy-phenyl)-3-methylsulfamoyl-benzamide | 320.37 | | 321.0 |
| 174 | 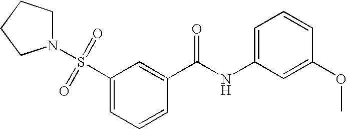 | 3-(Pyrrolidine-1-sulfonyl)-benzoic acid | 3-Methoxy-aniline | N-(3-Methoxy-phenyl)-3-(pyrrolidine-1-sulfonyl)-benzamide | 360.43 | | 361.1 |
| 175 | 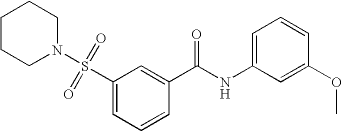 | 3-(Piperidine-1-sulfonyl)-benzoic acid | 3-Methoxy-aniline | N-(3-Methoxy-phenyl)-3-(piperidine-1-sulfonyl)-benzamide | 374.46 | | 375.3 |
| 176 | 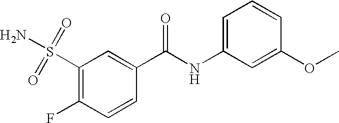 | 4-Fluoro-3-sulfamoyl-benzoic acid | 3-Methoxy-aniline | 4-Fluoro-N-(3-methoxy-phenyl)-3-sulfamoyl-benzamide | 324.33 | 323.3 | |

### EXAMPLE 177

### 3,4-Dichloro-N-[3-(2,5-dimethyl-imidazol-1-ylmethyl)-phenyl]-benzamide

### a) 2,5-Dimethyl-1-(3-nitro-benzyl)-1H-imidazole

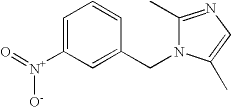

A solution of 300 mg (1.39 mmol) 3-nitrobenzyl bromide and 192 mg (1.39 mmol) 1-(2,4-dimethyl-imidazol-1-yl)-ethanone in 2 ml acetonitrile was stirred under microwave irradiation at 160° C. for 15 minutes. Then the reaction mixture was evaporated under reduced pressure, the residue taken up in 2M NaOH and heated to reflux for 15 min. Then the reaction mixture was extracted three times with dichloromethane. The combined organic extracts were washed with brine, dried over Na₂SO₄, filtered and evaporated. The crude product was purified by flash-chromatography on silica gel with a dichloromethane/methanol gradient with 5% to 10% methanol as eluent. 2,5-Dimethyl-1-(3-nitro-benzyl)-1H-imidazole was isolated as yellow liquid, MS (ISP): 231.9 ((M+H)^{+.}).

### b) 3-(2,5-Dimethyl-imidazol-1-ylmethyl)-phenylamine

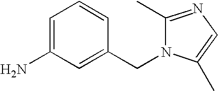

3-(2,5-Dimethyl-imidazol-1-ylmethyl)-phenylamine was prepared from 2,5-dimethyl-1-(3-nitro-benzyl)-1H-imidazole by catalytic hydrogenation with 10% Pd/C in ethyl acetate at ambient temperature for 3 hours: yellow solid, MS (ISP): 202.1 ((M+H)^{+.}).

### c) 3,4-Dichloro-N-[3-(2,5-dimethyl-imidazol-1-ylmethyl)-phenyl]-benzamide

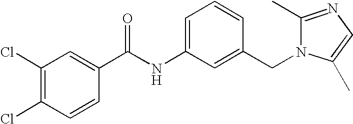

3,4-Dichloro-N-[3-(2,5-dimethyl-imidazol-1-ylmethyl)-phenyl]-benzamide was prepared in analogy to Example 1 from 3-(2,5-dimethyl-imidazol-1-ylmethyl)-phenylamine and 3,4-dichlorobenzoic acid: colorless solid, MS (ISP): 374.1 and 376.1 ((M+H)^{+.}).

### EXAMPLE 178

### 6-Chloro-N-(3-methoxy-phenyl)-5-trifluoromethyl-nicotinamide

### a) 1-Oxy-5-trifluoromethyl-pyridine-2,3-dicarboxylic acid diethyl ester

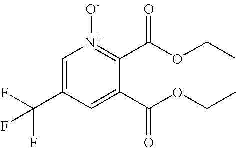

To a solution of 12.3 g (42 mmol) 5-frifluoromethyl-pyridine-2,3-dicarboxylic acid diethyl ester in 100 ml dichloromethane were added 8.34 g (89 mmol) hydrogen peroxide-urea adduct and the mixture cooled to 0° C. Drop-wise 11.75 ml (17.74 g, 84 mmol) trifluoroacetic acid anhydride were added and the mixture stirred at 0° C. for 3 hours. Then 25 ml 25% aqueous sodium sulfite solution were added and stirring continued for another 15 minutes. The mixture was poured onto 1N HCl and extracted twice with dichloromethane. The combined organic extracts were washed with brine, dried over Na₂SO₄, filtered and evaporated. The crude product was purified by flash-chromatography on silica gel with a heptane/ethyl acetate gradient with 0% to 50% ethyl acetate as eluent. 1-Oxy-5-trifluoromethyl-pyridine-2,3-dicarboxylic acid diethyl ester was isolated as colourless solid, MS (ISP): 308.1 ((M+H)^{+.}).

### b) 6-Chloro-5-trifluoromethyl-pyridine-2,3-dicarboxylic acid diethyl ester

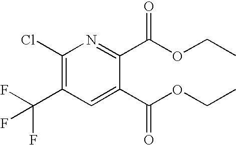

A solution of 11.0 g (36 mmol) 1-oxy-5-trifluoromethyl-pyridine-2,3-dicarboxylic acid diethyl ester in 33 ml (55 g, 360 mmol) phosphorous oxychloride was heated to reflux for 1 hour. Then the reaction mixture was evaporated under reduced pressure and the residue purified by flash-chromatography on silica gel with a heptane/ethyl acetate gradient with 5% to 20% ethyl acetate as eluent. 6-Chloro-5-trifluoromethyl-pyridine-2,3-dicarboxylic acid diethyl ester was isolated as colourless solid, MS (ISP): 326.3 and 328.4 ((M+H)^{+.}).

### c) 6-Chloro-5-trifluoromethyl-pyridine-2,3-dicarboxylic acid

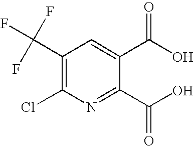

A solution of 9.60 g (29 mmol) 6-chloro-5-trifluoromethyl-pyridine-2,3-dicarboxylic acid diethyl ester in 30 ml tetrahydrofuran was cooled to 0° C. then 5 ml water and drop-wise 29.5 ml 2N NaOH. The stirred reaction mixture was allowed to come to ambient temperature within 30 minutes. Then the solution was saturated with sodium chloride and acidified with 2N HCl. The solution was extracted three times with ethyl acetate, the combined organic extracts washed with brine, dried over Na₂SO₄, filtered and evaporated. 6-Chloro-5-trifluoromethyl-pyridine-2,3-dicarboxylic acid was obtained as colourless solid, MS (ISN): 268.3 and 270.4 ((M−H)^{-.}).

### d) 6-Chloro-5-trifluoromethyl-nicotinic acid

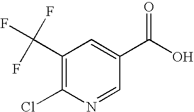

A solution of 400 mg (1.5 mmol) 6-chloro-5-trifluoromethyl-pyridine-2,3-dicarboxylic acid in 5 ml dioxane is heated under microwave irradiation to 165° C. for 15 minutes. The solvent was evaporated and the residue recrystallised from water. 6-Chloro-5-trifluoromethyl-nicotinic acid was obtained as colourless solid, MS (ISN): 224.0 and 226.1 ((M−H)^{-.}).

### e) 6-Chloro-N-(3-methoxy-phenyl)-5-trifluoromethyl-nicotinamide

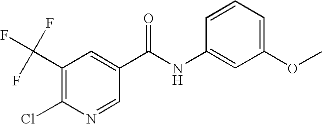

6-Chloro-N-(3-methoxy-phenyl)-5-trifluoromethyl-nicotinamide was prepared in analogy to Example 1 from 6-chloro-5-trifluoromethyl-nicotinic acid and 3-methoxy-aniline acid: colorless solid, MS (ISP): 374.1 and 376.1 ((M+H)^{+.}).

### EXAMPLE 179

### N-(3-Methoxy-phenyl)-6-pyrrolidin-1-yl-5-trifluoromethyl-nicotinamide

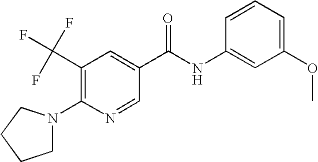

N-(3-Methoxy-phenyl)-6-pyrrolidin-1-yl-5-trifluoromethyl-nicotinamide was prepared in analogy to Example 143 from 6-chloro-N-(3-methoxy-phenyl)-5-trifluoromethyl-nicotinamide and pyrrolidine heated to 150° C. by microwave irradiation: colorless solid, MS (ISP): 366.0 ((M+H)^{+.}).

### EXAMPLE 180

### N-(3-Ethyl-phenyl)-4-pyrrolidin-1-yl-3-trifluoromethyl-benzamide

### a) 4-Pyrrolidin-1-yl-3-trifluoromethyl-benzoic acid

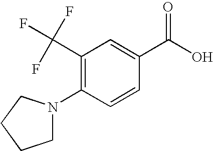

A solution of 1.00 g (4.8 mmol) 4-fluoro-3-(trifluoromethyl)-benzoic acid and 2.4 ml (2.06 g, 28.8 mmol) pyrrolidine in 3.8 ml dimethylsulfoxide was heated to 100° C. for 24 hours. The reaction mixture is cooled to ambient temperature, diluted with water and the pH adjusted to 3 with 4N HCR. The colourless precipitate was filtered, washed with water and dried: (2,4-Pyrrolidin-1-yl-3-trifluoromethyl-benzoic acid was obtained as slightly brown solid, MS (ISN): 258.0 ((M−H)^{-.}).

### b) N-(3-Ethyl-phenyl)-4-pyrrolidin-1-yl-3-trifluoromethyl-benzamide

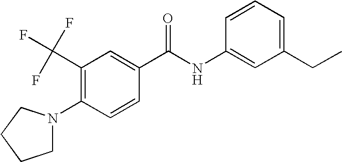

N-(3-Ethyl-phenyl)-4-pyrrolidin-1-yl-3-trifluoromethyl-benzamide was prepared in analogy to Example 1 from 4-pyrrolidin-1-yl-3-trifluoromethyl-benzoic acid and 3-ethyl-aniline: colorless solid, MS (ISP): 363.2 ((M+H)^{+.})

In analogy to Example 180 were prepared Examples 181 to 193:

| | | | | Chemical | | MS |
|---|---|---|---|---|---|---|
| | | | | Name of the | | (ISP): |
| Expl. | Structure | RCOOH | R′NHR″ | Product | MW | (M + H)⁺ |
| 181 | 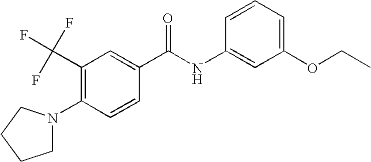 | 4-Pyrrolidin-1-yl-3-trifluoro-methyl-benzoic acid | 3-Ethoxy-aniline | N-(3-Ethoxy-phenyl)-4-pyrrolidin-1-yl-3-trifluoro-methyl-benzamide | 378.39 | 379.3 |
| 182 | 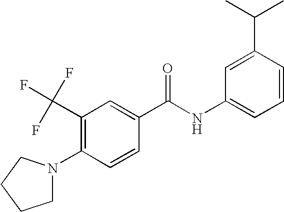 | 4-Pyrrolidin-1-yl-3-trifluoro-methyl-benzoic acid | 3-iso-Propyl-aniline | N-(3-Isopropyl-phenyl)-4-pyrrolidin-1-yl-3-trifluoro-methyl-benzamide | 376.42 | 377.3 |
| 183 | 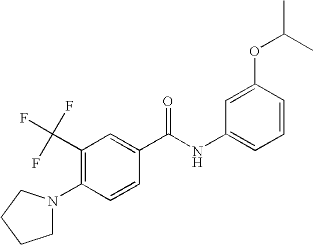 | 4-Pyrrolidin-1-yl-3-trifluoro-methyl-benzoic acid | 3-iso-Propoxy-aniline | N-(3-Isopropoxy-phenyl)-4-pyrrolidin-1-yl-3-trifluoro-methyl-benzamide | 392.42 | 393.1 |
| 184 | 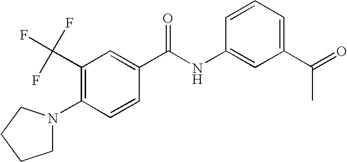 | 4-Pyrrolidin-1-yl-3-trifluoro-methyl-benzoic acid | 1-(3-Amino-phenyl)-ethanone | N-(3-Acetyl-phenyl)-4-pyrrolidin-1-yl-3-tri-fluoromethyl-benzamide | 376.38 | 377.3 |
| 185 | 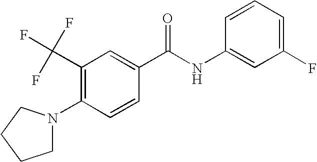 | 4-Pyrrolidin-1-yl-3-trifluoro-methyl-benzoic acid | 3-Fluoro-aniline | N-(3-Fluoro-phenyl)-4-pyrrolidin-1-yl-3-trifluoro-methyl-benzamide | 352.33 | 353.1 |
| 186 | 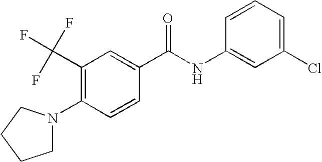 | 4-Pyrrolidin-1-yl-3-trifluoro-methyl-benzoic acid | 3-Chloro-aniline | N-(3-Chloro-phenyl)-4-pyrrolidin-1-yl-3-trifluoro-methyl-benzamide | 368.79 | 369.0and371.1 |
| 187 | 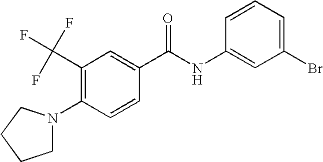 | 4-Pyrrolidin-1-yl-3-trifluoro-methyl-benzoic acid | 3-Bromo-aniline | N-(3-Bromo-phenyl)-4-pyrrolidin-1-yl-3-trifluoro-methyl-benzamide | 413.24 | 413.1and415.1 |
| 188 | 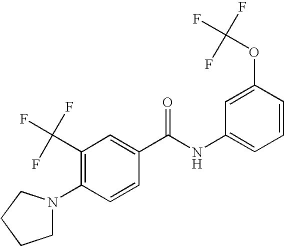 | 4-Pyrrolidin-1-yl-3-trifluoro-methyl-benzoic acid | 3-Trifluoro-methoxy-aniline | 4-Pyrrolidin-1-yl-N-(3-trifluoro-methoxy-phenyl)-3-trifluoro-methyl-benzamide | 418.34 | 419.3 |
| 189 | 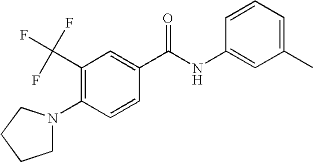 | 4-Pyrrolidin-1-yl-3-trifluoro-methyl-benzoic acid | 3-Methyl-aniline | 4-Pyrrolidin-1-yl-N-m-tolyl-3-trifluoro-methyl-benzamide | 348.37 | 349.3 |
| 190 | 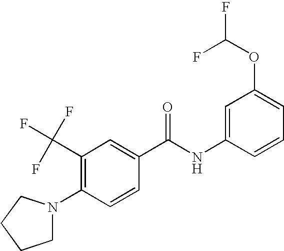 | 4-Pyrrolidin-1-yl-3-trifluoro-methyl-benzoic acid | 3-Difluoro-methoxy-aniline | N-(3-Difluoro-methoxy-phenyl)-4-pyrrolidin-1-yl-3-trifluoro-methyl-benzamide | 400.35 | 401.4 |
| 191 | 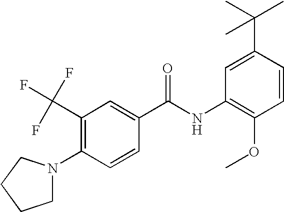 | 4-Pyrrolidin-1-yl-3-trifluoro-methyl-benzoic acid | 5-tert-Butyl-2-methoxy-aniline | N-(5-tert-Butyl-2-methoxy-phenyl)-4-pyrrolidin-1-yl-3-trifluoro-methyl-benzamide | 420.00 | 421.1 |
| 192 | 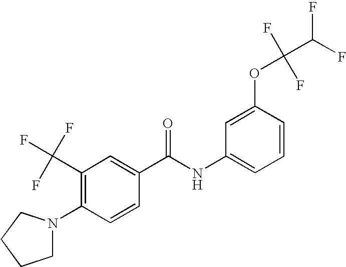 | 4-Pyrrolidin-1-yl-3-trifluoro-methyl-benzoic acid | 3-(1,1,2,2-Tetra-fluoro-ethoxy)-aniline | 4-Pyrrolidin-1-yl-N-[3-(1,1,2,2-tetrafluoro-ethoxy)-phenyl]-3-trifluoro-methyl-benzamide | 450.35 | 451.2 |
| 193 | 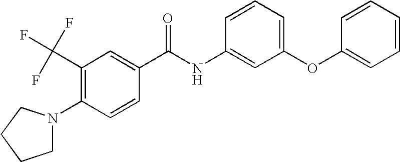 | 4-Pyrrolidin-1-yl-3-trifluoro-methyl-benzoic acid | 3-Phenoxy-aniline | N-(3-Phenoxy-phenyl)-4-pyrrolidin-1-yl-3-trifluoro-methyl-benzamide | 426.44 | 427.2 |

### EXAMPLE 194

### (rac,meso)-4-(3,5-Dimethyl-piperidin-1-yl)-N-(3-methoxy-phenyl)-3-trifluoromethyl-benzamide

### a) (rac,meso)-4-(3,5-Dimethyl-piperidin-1-yl)-3-trifluoromethyl-benzoic acid

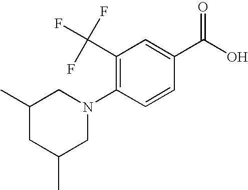

(rac,meso)-4-(3,5-Dimethyl-piperidin-1-yl)-3-trifluoromethyl-benzoic acid was prepared in analogy to Example 180 a) from 4-fluoro-3-(trifluoromethyl)-benzoic acid and (rac,meso)-3,5-dimethyl-piperidine: colorless solid, MS (ISN): 300.5 ((M−H)^{-.}).

### b) (rac,meso)-4-(3,5-Dimethyl-piperidin-1-yl)-N-(3-methoxy-phenyl)-3-trifluoromethyl-benzamide

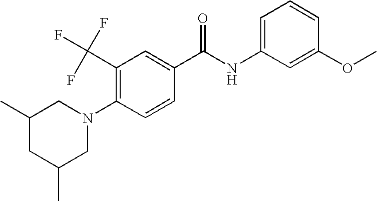

(rac,meso)-4-(3,5-Dimethyl-piperidin-1-yl)-N-(3-methoxy-phenyl)-3-trifluoromethyl-benzamide was prepared in analogy to Example 1 from (rac,meso)-4-(3,5-dimethyl-piperidin-1-yl)-3-trifluoromethyl-benzoic acid and 3-methoxy-aniline: colorless solid, MS (ISP): 407.5 ((M+H)^{+.}).

### EXAMPLE 195

### 4-Azepan-1-yl-N-(3-methoxy-phenyl)-3-trifluoromethyl-benzamide

### a) 4-Azepan-1-yl-3-trifluoromethyl-benzoic acid

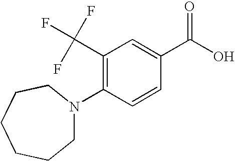

4-Azepan-1-yl-3-trifluoromethyl-benzoic acid was prepared in analogy to Example 180 a) from 4-fluoro-3-(trifluoromethyl)-benzoic acid and azepane: colorless solid, MS (ISN): 286.4 ((M−H)^{-.}).

### b) 4-Azepan-1-yl-N-(3-methoxy-phenyl)-3-trifluoromethyl-benzamide

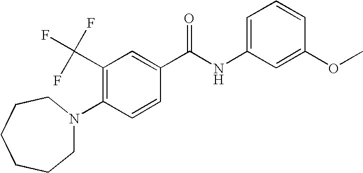

4-Azepan-1-yl-N-(3-methoxy-phenyl)-3-trifluoromethyl-benzamide was prepared in analogy to Example 1 from 4-azepan-1-yl-3-trifluoromethyl-benzoic acid and 3-methoxy-aniline: colorless solid, MS (ISP): 393.0 ((M+H)^{+.}).

### EXAMPLE 196

### 4-(4-Cyano-piperidin-1-yl)-N-(3-methoxy-phenyl)-3-trifluoromethyl-benzamide

### a) 4-(4-Cyano-piperidin-1-yl)-3-trifluoromethyl-benzoic acid

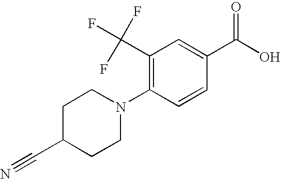

4-(4-Cyano-piperidin-1-yl)-3-trifluoromethyl-benzoic acid was prepared in analogy to Example 180 a) from 4-fluoro-3-(trifluoromethyl)-benzoic acid and piperidine-4-carbonitrile: colorless solid, MS (ISN): 297.5 ((M−H)^{-.}).

### b) 4-(4-Cyano-piperidin-1-yl)-N-(3-methoxy-phenyl)-3-trifluoromethyl-benzamide

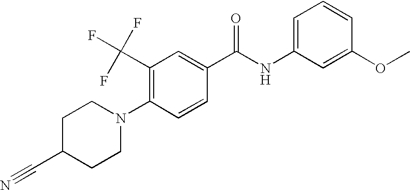

4-(4-Cyano-piperidin-1-yl)-N-(3-methoxy-phenyl)-3-trifluoromethyl-benzamide was prepared in analogy to Example 1 from 4-(4-cyano-piperidin-1-yl)-3-trifluoromethyl-benzoic acid and 3-methoxy-aniline: colorless solid, MS (ISP): 404.4 ((M+H)^{+.}).

### EXAMPLE 197

### N-(3-Methoxy-phenyl)-3-trifluoromethyl-4-(4-trifluoromethyl-piperidin-1-yl)-benzamide

### a) 3-Trifluoromethyl-4-(4-trifluoromethyl-piperidin-1-yl)-benzoic acid

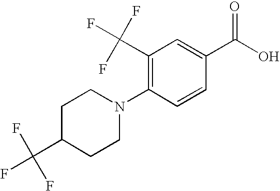

3-Trifluoromethyl-4-(4-trifluoromethyl-piperidin-1-yl)-benzoic acid was prepared in analogy to Example 180 a) from 4-fluoro-3-(trifluoromethyl)-benzoic acid and 4-trifluoromethyl-piperidine: colorless solid, MS (ISN): 340.3 ((M−H)^{-.}).

### b) N-(3-Methoxy-phenyl)-3-trifluoromethyl-4-(4-trifluoromethyl-piperidin-1-yl)-benzamide

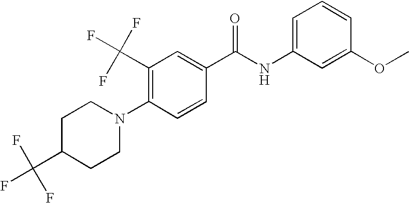

N-(3-Methoxy-phenyl)-3-trifluoromethyl-4-(4-trifluoromethyl-piperidin-1-yl)-benzamide was prepared in analogy to Example 1 from 3-trifluoromethyl-4-(4-trifluoromethyl-piperidin-1-yl)-benzoic acid and 3-methoxy-aniline: colorless solid, MS (ISP): 447.1 ((M+H)^{+.}).

### EXAMPLE 198

### 2-Pyrrolidin-1-yl-pyrimidine-5-carboxylic acid (3-methoxy-phenyl)-amide

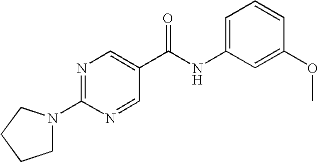

2-Pyrrolidin-1-yl-pyrimidine-5-carboxylic acid (3-methoxy-phenyl)-amide was prepared in analogy to Example 1 from 2-pyrrolidin-1-yl-pyrimidine-5-carboxylic acid and 3-methoxy-aniline: colorless solid, MS (ISP): 299.0 ((M+H)^{+.}).

In analogy to examples 143 were prepared examples 199 to 209:

| Ex- | | | | Rxn | Rxn | | MS |
|---|---|---|---|---|---|---|---|
| am- | | 6-chloro- | | Temp. | time | | (ISN): |
| ple | Structure | nicotinamide | R′NHR″ | (° C.) | (min.) | MW | (M − H)⁻ |
| 199 | 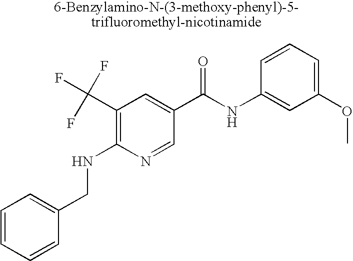 | 6-Chloro-N-(3-methoxy-phenyl)-5-trifluoromethyl-nicotinamide | Benzyl-amine | 250 | 15 | 401.39 | 400.3 |
| 200 | 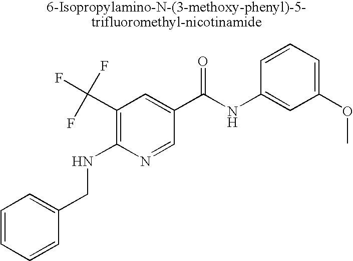 | 6-Chloro-N-(3-methoxy-phenyl)-5-trifluoromethyl-nicotinamide | iso-Propyl-amine | 250 | 15 | 353.34 | 352.2 |
| 201 | 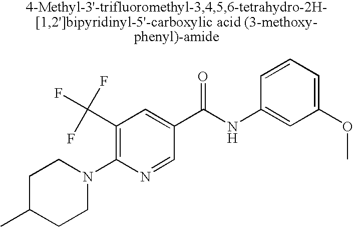 | 6-Chloro-N-(3-methoxy-phenyl)-5-trifluoromethyl-nicotinamide | 4-Methyl-piperi-dine | 250 | 15 | 393.41 | 392.1 |
| 202 | 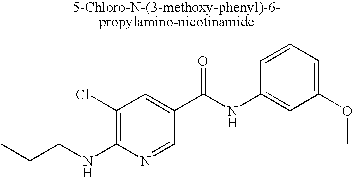 | 5,6-Dichloro-N-(3-methoxy-phenyl)-nicotinamide | Propyl-amine | 180 | 15 | 319.79 | |
| 203 | 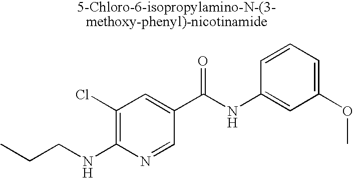 | 5,6-Dichloro-N-(3-methoxy-phenyl)-nicotinamide | iso-Propyl-amine | 180 | 15 | 319.79 | |
| 204 | 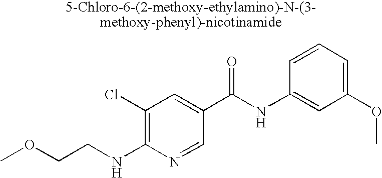 | 5,6-Dichloro-N-(3-methoxy-phenyl)-nicotinamide | 2-Meth-oxy-ethylamine | 180 | 15 | 335.79 | |
| 205 | 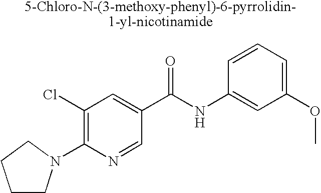 | 5,6-Dichloro-N-(3-methoxy-phenyl)-nicotinamide | Pyrrol-idine | 180 | 15 | 331.80 | |
| 206 | 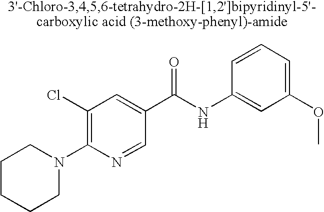 | 5,6-Dichloro-N-(3-methoxy-phenyl)-nicotinamide | Piperi-dine | 250 | 15 | 345.83 | |
| 207 | 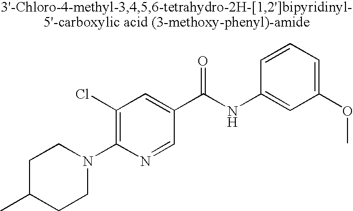 | 5,6-Dichloro-N-(3-methoxy-phenyl)-nicotinamide | 4-Methyl-piperi-dine | 250 | 15 | 359.86 | |
| 208 | 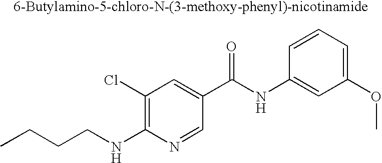 | 5,6-Dichloro-N-(3-methoxy-phenyl)-nicotinamide | Butyl-amine | 180 | 15 | 333.82 | |
| 209 | 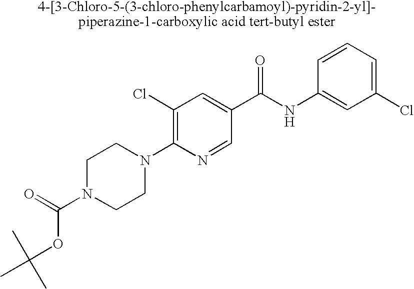 | 5,6-Dichloro-N-(3-methoxy-phenyl)-nicotinamide | Piper-azine-1-car-boxylicacid tert-butylester | 120 | 15 | 451.35 | |

### EXAMPLE 210

### 5-Chloro-N-(3-chloro-phenyl)-6-piperazin-1-yl-nicotinamide

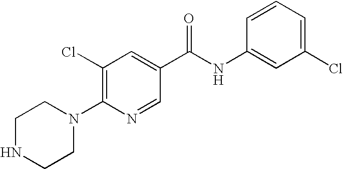

To a solution of 200 mg (0.443 mmol) 4-[3-chloro-5-(3-chloro-phenylcarbamoyl)-pyridin-2-yl]-piperazine-1-carboxylic acid tert-butyl ester (Example 209) in 2 ml ethanol were added 2 ml aqueous 1N HCR and the mixture stirred at 80° C. for 1.5 hours. Then the mixture was cooled to ambient temperature neutralized with 2N NaOH and extracted with dichloromethane. The combined organic extracts were washed with brine, dried over Na₂SO₄, filtered and evaporated. 5-Chloro-N-(3-chloro-phenyl)-6-piperazin-1-yl-nicotinamide was obtained as a colourless solid: MS (ISP): 351.2 and 353.2 ((M+H)^{+.}).

### EXAMPLE 211

### 3-(1-Butoxy-vinyl)-4-fluoro-N-(3-methoxy-phenyl)-benzamide

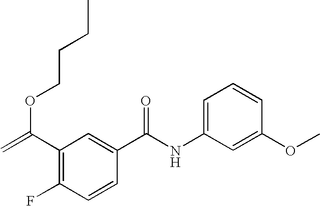

A mixture of 300 mg (0.925 mmol) 3-bromo-4-fluoro-N-(3-methoxy-phenyl)-benzamide (Example 71), 8 mg (0.037 mmol) Pd(II) acetate, 31 mg (0.075 mmol) 1,3-bis(diphenylphosphino)propane in 2 ml DMSO and 0.2 ml 1-butyl-3-methylimidazolium tetrafluoroborate was stirred at ambient temperature and degassed 3 times. To the mixture were added 185 mg (0.24 ml, 1.85 mmol) N-butyl vinyl ether and 112 mg (0.16 ml, 1.11 mmol) diisopropylamine and the sealed tube stirred under microwave irradiation at 170° C. for 15 minutes. The resulting reaction mixture was evaporated and the residue purified by flash-chromatography on silica gel with heptane/ethyl acetate 78:22 as eluent. 3-(1-Butoxy-vinyl)-4-fluoro-N-(3-methoxy-phenyl)-benzamide was obtained as yellow oil: MS (ISP): 344.2 ((M+H)^{+.}).

### EXAMPLE 212

### 6-(5,6-Dihydro-4H-pyran-2-yl)-N-(3-methoxy-phenyl)-nicotinamide

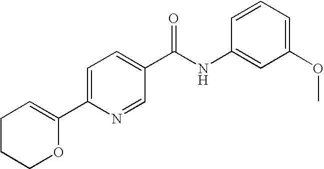

To a solution of 140 mg (0.533 mmol) 6-chloro-N-(3-methoxy-phenyl)-nicotinamide (Example 277) in 4 ml dioxane were added 20 mg (0.086 mmol) tri(2-furyl)phosphine, 9 mg (0.016 mmol) bis(benzylidenacetone) palladium and 80.8 mg (111 ul, 0.800 mmol) triethylamine and stirred at ambient temperature for 10 min. Then 298 mg (0.800 mmol) tributyl-(5,6-dihydro-4H-pyran-2-yl)-stannane were added and the mixture heated to 110° C. for 24 hours. The cooled reaction mixture was filtered through Dicalite, the filtrate diluted with ethyl acetate and extracted with water. The organic phase was washed with brine, dried over Na₂SO₄, filtered and evaporated. The residue was purified by flash-chromatography on silica gel with a gradient of heptane/ethyl acetate 60:40 to 40:60 as eluent. 6-(5,6-Dihydro-4H-pyran-2-yl)-N-(3-methoxy-phenyl)-nicotinamide was obtained as a colourless solid: MS (ISP): 311.1 ((M+H)^{+.}).

### EXAMPLE 213

### 3-Acetyl-4-fluoro-N-(3-methoxy-phenyl)-benzamide

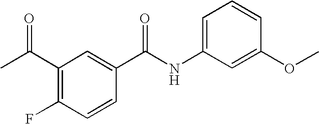

To a solution of 90 mg (0.26 mmol) 3-(1-butoxy-vinyl)-4-fluoro-N-(3-methoxy-phenyl)-benzamide (Example 211) in 2 ml dioxane were added 2 ml 2N HCl and the mixture stirred at ambient temperature for 30 minutes. The reaction mixture was extracted with with dichloromethane. The combined organic extracts were washed with brine, dried over Na₂SO₄, filtered and evaporated. The residue was purified by flash-chromatography on silica gel with heptane/ethyl acetate 70:30 as eluent. 3-Acetyl-4-fluoro-N-(3-methoxy-phenyl)-benzamide was obtained as a colourless solid: MS (ISN): 286.1 ((M−H)^{-.}).

### EXAMPLE 214

### rac-N-(3-Methoxy-phenyl)-6-(tetrahydro-pyran-2-yl)-nicotinamide

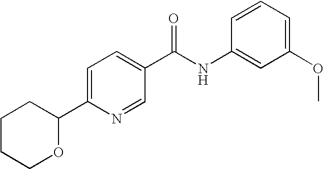

To a solution of 30 mg (0.097 mmol) 6-(5,6-dihydro-4H-pyran-2-yl)-N-(3-methoxy-phenyl)-nicotinamide in 3 ml ethyl acetate was added a tip of a spatula platinum oxide and the mixture stirred under a hydrogen atmosphere at ambient temperature for 1 hour. Then the reaction mixture is filtered, evaporated and the residue was purified by flash-chromatography on silica gel with heptane/ethyl acetate 1:2 as eluent. rac-N-(3-Methoxy-phenyl)-6-(tetrahydro-pyran-2-yl)-nicotinamide was obtained as a colourless solid: MS (ISN): 313.0 ((M−H)^{-.}).

### EXAMPLE 215

### rac-4-Fluoro-N-(3-methoxy-phenyl)-3-(tetrahydro-furan-2-yl)-benzamide

### a) 3-(4,5-Dihydro-furan-2-yl)-4-fluoro-N-(3-methoxy-phenyl)-benzamide

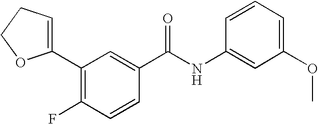

3-(4,5-Dihydro-furan-2-yl)-4-fluoro-N-(3-methoxy-phenyl)-benzamide was prepared in analogy to Example 212 from 6-chloro-N-(3-methoxy-phenyl)-nicotinamide (Example 277) and tributyl-(4,5-dihydro-furan-2-yl)-stannane under microwave irradiation at 170° C. for 15 minutes: viscous colorless oil, MS (ISP): 314.1 ((M+H)^{+.}).

### b) rac-4-Fluoro-N-(3-methoxy-phenyl)-3-(tetrahydro-furan-2-yl)-benzamide

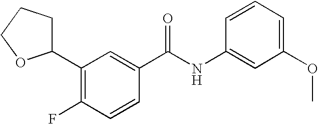

rac-4-Fluoro-N-(3-methoxy-phenyl)-3-(tetrahydro-furan-2-yl)-benzamide was prepared in analogy to Example 214 from 3-(4,5-dihydro-furan-2-yl)-4-fluoro-N-(3-methoxy-phenyl)-benzamide: viscous colorless oil, MS (ISP): 316.0 ((M+H)^{+.}).

### EXAMPLE 216

### 4-Chloro-N-(3-methoxy-phenyl)-3-trifluoromethyl-benzamide

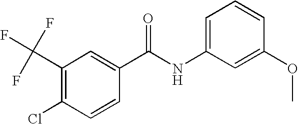

4-Chloro-N-(3-methoxy-phenyl)-3-trifluoromethyl-benzamide was prepared in analogy to Example 1 from 3-methoxy-aniline and 4-chloro-3-trifluoromethyl-benzoic acid: beige solid, MS (ISN): 328.0 ((M−H)^{-.}).

### Known Compounds

### EXAMPLES 217 TO 333

The following are known compounds, and they are commercially available or disclosed in the references.

| Example | Structure | Chemical name | Reference |
|---|---|---|---|
| 217 | 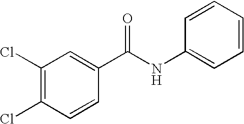 | 3,4-Dichloro-N-phenyl-benzamide | Monatshefte fuerChemie (1966), 97(1),271-80. |
| 218 | 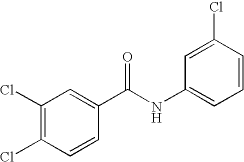 | 3,3′,4-Trichlorobenzanilide | CH 609558 |
| 219 | 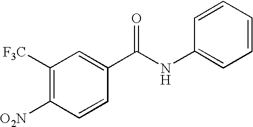 | 3-Trifluoromethyl-4-nitro-N-phenyl-benzamide | Macromolecular RapidCommunications(2002), 23(12),665-671. |
| 220 | 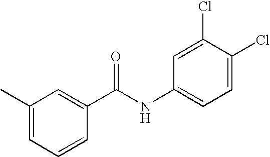 | N-(3,4-Dichloro-phenyl)-3-methyl-benzamide | Journal of Agriculturaland Food Chemistry(1986), 34 (4), 725-32. |
| 221 | 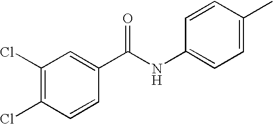 | 3,4-Dichloro-N-p-tolyl-benzamide | Monatshefte fuerChemie (1966), 97(1),271-80. |
| 222 | 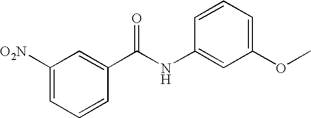 | N-(3-Methoxy-phenyl)-3-nitro-benzamide | Journal ofCombinatorialChemistry (2002), 4(6),549-551. |
| 223 | 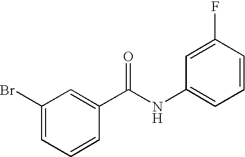 | 3-Bromo-N-(3-fluoro-phenyl)-benzamide | Pharmazie (1998),53(3), 193-195. |
| 224 | 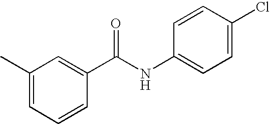 | N-(4-Chloro-phenyl)-3-methyl-benzamide | Bulletin de la SocieteChimique de France(1963), (4), 862-72. |
| 225 | 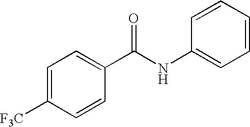 | N-Phenyl-4-trifluoromethyl-benzamide | Journal of OrganicChemistry (1996),61(21), 7482-7485. |
| 226 | 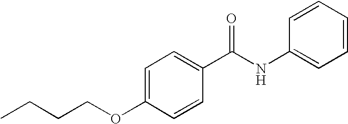 | 4-Butoxy-N-phenyl-benzamide | Journal of the Chemicalsociety (1949), 3043-6. |
| 227 | 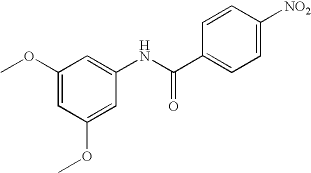 | N-(3,5-Dimethoxy-phenyl)-4-nitro-benzamide | Journal of MedicinalChemistry (1996),39(17), 3375-3384. |
| 228 | 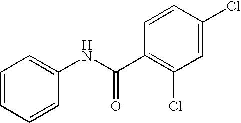 | 2,4-Dichloro-N-phenyl-benzamide | Journal of OrganicChemistry (1983),48(23), 4391-3. |
| 229 | 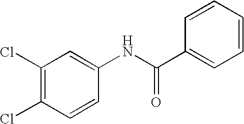 | N-(3,4-Dichloro-phenyl)-benzamide | Helvetica Chimica Acta(1964), 47(1), 162-5. |
| 230 | 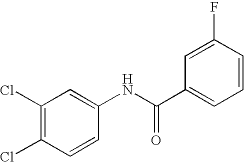 | N-(3,4-Dichloro-phenyl)-3-fluoro-benzamide | Agricultural andBiological Chemistry(1976), 40(1), 213-14. |
| 231 | 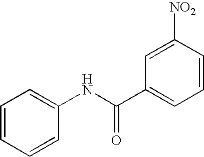 | 3-Nitro-N-phenyl-benzamide | Journal of MedicinalChemistry (1986),29(8), 1534-7. |
| 232 | 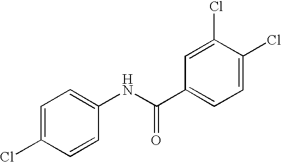 | 3,4-Dichloro-N-(4-chloro-phenyl)-benzamide | Journal of the AmericanChemical Society(1957), 79 1236-45. |
| 233 | 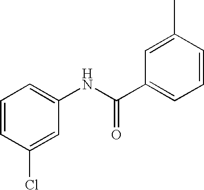 | N-(3-Chloro-phenyl)-3-methyl-benzamide | Chemische Berichte(1990), 123(11),2191-4. |
| 234 | 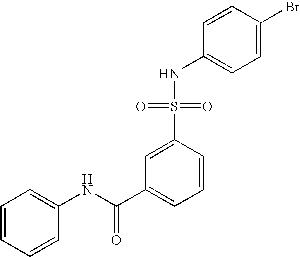 | 3-(4-Bromo-phenylsulfamoyl)-N-phenyl-benzamide | WO 2005087217 |
| 235 | 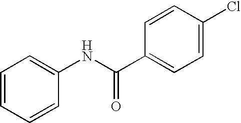 | 4-Chlorobenzanilide | Chemische Berichte(1964), 97(2), 472-9. |
| 236 | 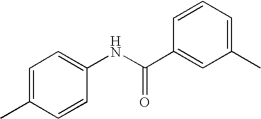 | 3-Methyl-N-p-tolyl-benzamide | US 2004235888 |
| 237 | 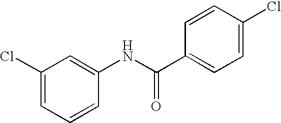 | 4-Chloro-N-(3-chloro-phenyl)-benzamide | Chemische Berichte(1990), 123(11),2191-4. |
| 238 | 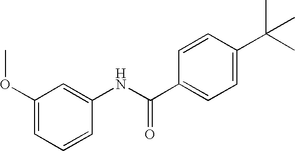 | 4-tert-Butyl-N-(3-methoxy-phenyl)-benzamide | DE 3830054 |
| 239 | 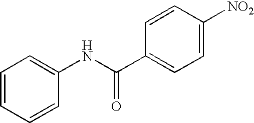 | N-Phenyl-4-nitrobenzenecarboxamide | Journal of OrganicChemistry (2006),71(9), 3375-3380. |
| 240 | 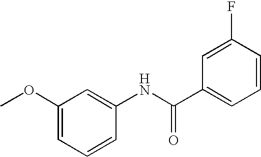 | 3-Fluoro-N-(3-methoxy-phenyl)-benzamid | Magnetic Resonance inChemistry (1997),35(8), 543-548. |
| 241 | 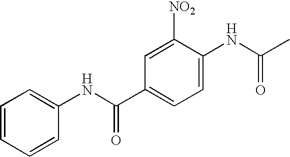 | 4-Acetylamino-3-nitro-N-phenyl-benzamide | Journal of MedicinalChemistry (1984),27(8), 1083-9. |
| 242 | 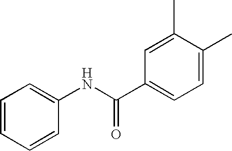 | 3,4-dimethyl-N-phenylbenzamide | Journal of the ChemicalSociety (1931), 2323-31. |
| 243 | 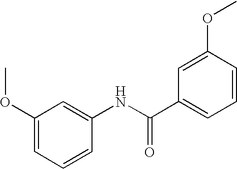 | 3-Methoxy-N-(3-methoxy-phenyl)-benzamide | Journal of OrganicChemistry (1958), 23,349-53. |
| 244 | 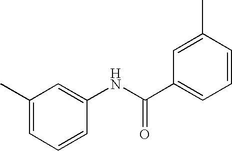 | 3-Methyl-N-m-tolyl-benzamide | Helvetica Chimica Acta(1963), 46(4), 1148-50. |
| 245 | 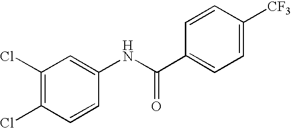 | N-(3,4-Dichloro-phenyl)-4-trifluoromethyl-benzamide | CH 609558 |
| 246 | 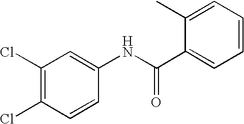 | N-(3,4-Dichloro-phenyl)-2-methyl-benzamide | Pesticide Biochemistryand Physiology (1989),34(3), 255-76. |
| 247 | 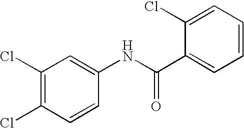 | 2-Chloro-N-(3,4-dichloro-phenyl)-benzamide | Zhurnal ObshcheiKhimii (1966), 36(4),638-9. |
| 248 | 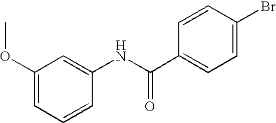 | 4-Bromo-N-(3-methoxy-phenyl)-benzamide | Journal of the ChemicalSociety, Transactions(1925), 127, 990-5. |
| 249 | 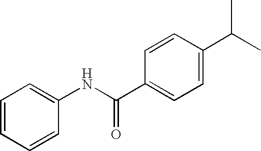 | 4-Isopropyl-N-phenyl-benzamide | Helvetica Chimica Acta(1958), 41, 1606-32. |
| 250 | 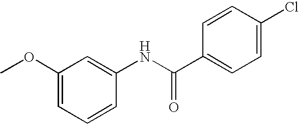 | 4-Chloro-N-(3-methoxy-phenyl)-benzamide | Taehan Hwahakhoe Chi(1972), 17(3), 193-7. |
| 251 | 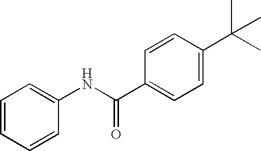 | 4-tert-Butyl-N-phenyl-benzamide | Nippon NoyakuGakkaishi (1985), 10(4),697-702. |
| 252 | 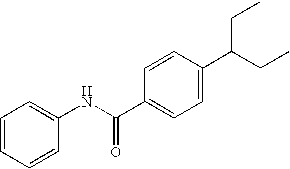 | 4-Diethylamino-N-phenyl-benzamide | Tetrahedron Letters(1971), (4), 321-2. |
| 253 | 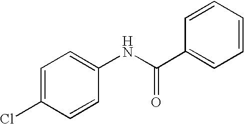 | N-(4-Chloro-phenyl)-benzamide | Journal of MedicinalChemistry (1989),32(5), 1033-8. |
| 254 | 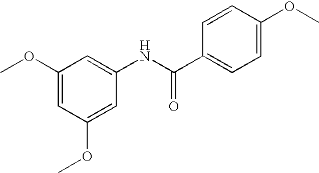 | N-(3,5-Dimethoxy-phenyl)-4-methoxy-benzamide | Monatshefte fuerChemie (1931), 5763-70. |
| 255 | 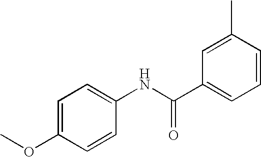 | N-(4-Methoxy-phenyl)-3-methyl-benzamide | Journal ofCombinatorialChemistry (2002),4(6), 549-551. |
| 256 | 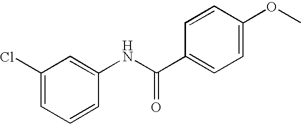 | N-(3-Chloro-phenyl)-4-methoxy-benzamide | Journal of PhysicalOrganic Chemistry(1994), 7(6), 273-9. |
| 257 | 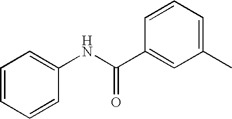 | 3-Methyl-N-phenyl-benzamide | Chemische Berichte(1990), 123(11),2191-4. |
| 258 | 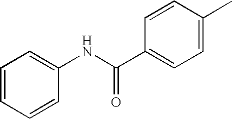 | 4-Methyl-N-phenyl-benzamide | Chemische Berichte(1990), 123(11),2191-4. |
| 259 | 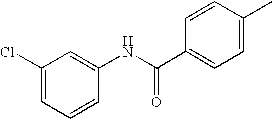 | N-(3-Chloro-phenyl)-4-methyl-benzamide | Journal of PhysicalOrganic Chemistry(1994), 7(6), 273-9. |
| 260 | 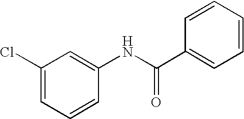 | N-(3-Chloro-phenyl)-benzamide | Journal of PhysicalOrganic Chemistry(1994), 7(6), 273-9. |
| 261 | 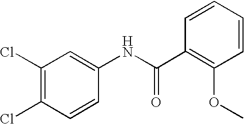 | N-(3,4-Dichloro-phenyl)-2-methoxy-benzamide | Archiv der Pharmazie(Weinheim, Germany)(1988), 321(7), 419-22. |
| 262 | 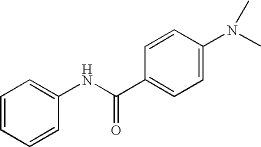 | 4-Dimethylamino-N-phenyl-benzamide | Helvetica ChimicaActa (1919), 2, 717-9. |
| 263 | 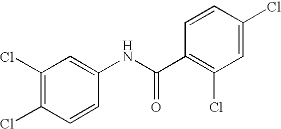 | 2,4-Dichloro-N-(3,4-dichloro-phenyl)-benzamide | Journal of Pharmacyand Pharmacology(1964), 16(3), 163-73. |
| 264 | 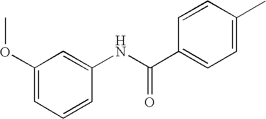 | N-(3-Methoxy-phenyl)-4-methyl-benzamide | Journal of MedicinalChemistry (1986),29(5), 820-5. |
| 265 | 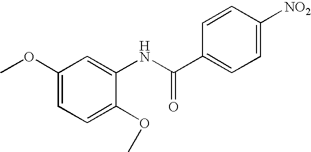 | N-(2,5-Dimethoxy-phenyl)-4-nitro-benzamide | EP 661266 |
| 266 | 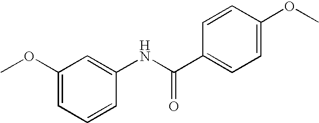 | 4-Methoxy-N-(3-methoxy-phenyl)-benzamide | Australian Journal ofChemistry (1984),37(4), 831-44. |
| 267 | 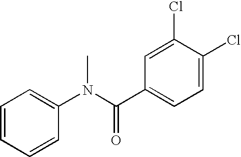 | 3,4-Dichloro-N-methyl-N-phenyl-benzamide | Farmaco, EdizioneScientifica (1969),24(12), 1025-37. |
| 268 | 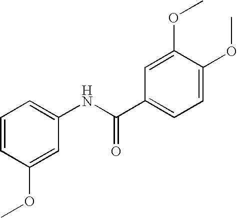 | 3,4-Dimethoxy-N-(3-methoxy-phenyl)-benzamide | Bulletin de la SocieteChimique de France(1965), (3), 848-58. |
| 269 | 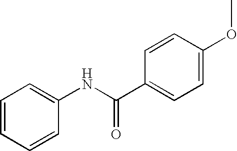 | 4-Methoxy-N-phenyl-benzamide | Journal of PhysicalOrganic Chemistry(1994), 7(6), 273-9. |
| 270 | 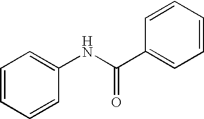 | N-Phenyl-benzamide | Journal of PhysicalOrganic Chemistry(1994), 7(6), 273-9. |
| 271 | 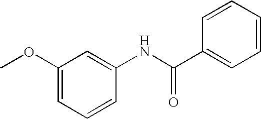 | N-(3-Methoxy-phenyl)-benzamide | Journal of the AmericanChemical Society(1958), 80 3328-32. |
| 272 | 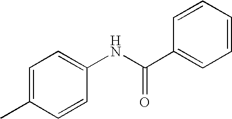 | N-p-Tolyl-benzamide | Journal of PhysicalOrganic Chemistry(1994), 7(6), 273-9. |
| 273 | 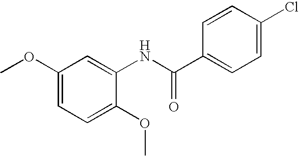 | 4-Chloro-N-(2,5-dimethoxy-phenyl)-benzamide | Perkin 1 (2000), (2),205-210. |
| 274 | 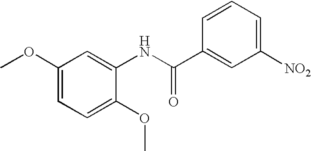 | N-(2,5-Dimethoxy-phenyl)-3-nitro-benzamide | EP 440195 |
| 275 | 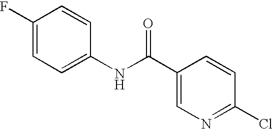 | 6-Chloro-N-(4-fluoro-phenyl)-nicotinamide | WO02/053544 |

| | | | Reference |
|---|---|---|---|
| Example | Structure | Chemical name | (SciFinder) |
| 276 | N-Phenyl-benzamide | 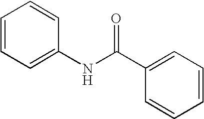 | 93-98-1 |
| 277 | N-(4-Chloro-phenyl)-benzamide | 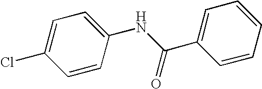 | 2866-82-2 |
| 278 | 4-Methoxy-N-phenyl-benzamide | 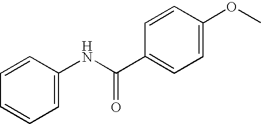 | 7465-88-5 |
| 279 | 4-Methyl-N-phenyl-benzamide | 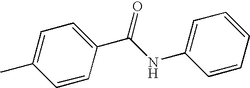 | 6833-18-7 |
| 280 | 4-Chloro-N-phenyl-benzamide | 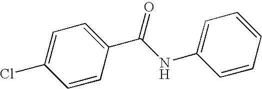 | 6833-15-4 |
| 281 | 4-Chloro-N-(3-chloro-phenyl)-benzamide | 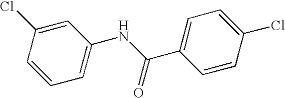 | 2447-96-3 |
| 282 | 3,4-Dichloro-N-(4-chloro-phenyl)-benzamide | 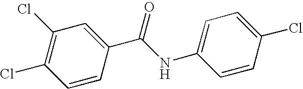 | 06.04.2448 |
| 283 | 3,4-Dichloro-N-(3-chloro-phenyl)-benzamide | 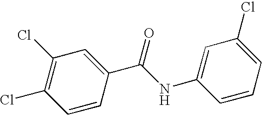 | 05.03.2448 |
| 284 | N-(3,4-Dichloro-phenyl)-4-trifluoromethyl-benzamide | 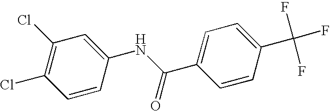 | 56661-54-2 |
| 285 | Ethyl p-(2,3,4,5-tetrahydro-1-benzothiepin-7-carboxamido)benzoate | 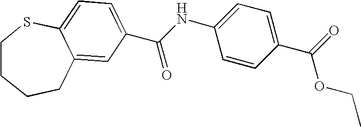 | 129790-95-0 |
| 286 | 4-Methoxy-N-(3-methoxy-phenyl)-benzamide | 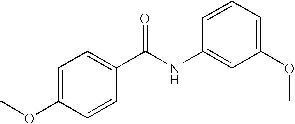 | 91099-22-8 |
| 287 | N-(3-Chloro-phenyl)-4-methoxy-benzamide | 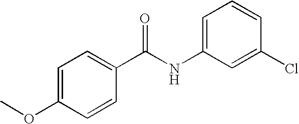 | 7465-93-2 |
| 288 | N-(3-Methoxy-phenyl)-benzamide | 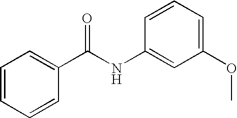 | 13031-49-7 |
| 289 | 4-Nitro-N-phenyl-benzamide | 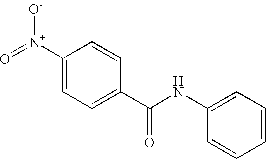 | 3460-11-5 |
| 290 | 4-tert-Butyl-N-(3-methoxy-phenyl)-benzamide | 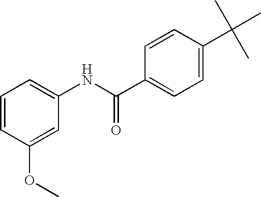 | 129504-97-8 |
| 291 | N-(3,5-Dimethoxy-phenyl)-4-nitro-benzamide | 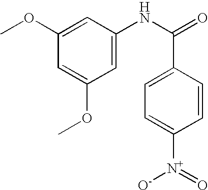 | 152586-91-9 |
| 292 | 3-Fluoro-N-(3-methoxy-phenyl)-benzamide | 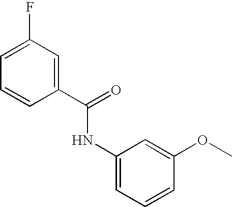 | 195378-99-5 |
| 293 | N-(3,4-Dichloro-phenyl)-2-methoxy-benzamide | 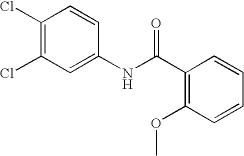 | 117367-18-7 |
| 294 | N-(3,5-Dimethoxy-phenyl)-4-methoxy-benzamide | 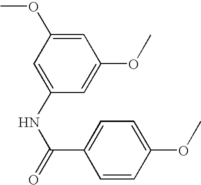 | 134029-84-8 |
| 295 | N-(4-Chloro-phenyl)-3-methyl-benzamide | 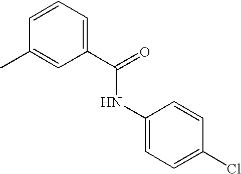 | 81636-14-8 |
| 296 | 3-Methyl-N-m-tolyl-benzamide | 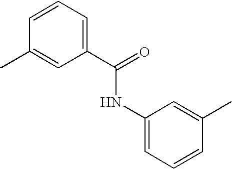 | 53205-69-9 |
| 297 | 3-Bromo-N-(3-fluoro-phenyl)-benzamide | 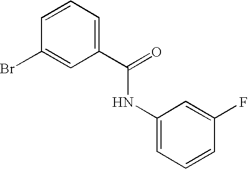 | 206062-09-1 |
| 298 | N-(3-Chloro-phenyl)-3-methyl-benzamide | 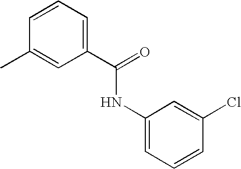 | 96749-32-5 |
| 299 | N-(3,4-Dichloro-phenyl)-3-methyl-benzamide | 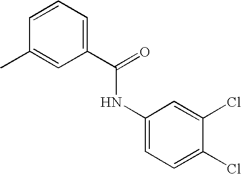 | 102587-39-3 |
| 300 | N-(2,5-Dimethoxy-phenyl)-4-nitro-benzamide | 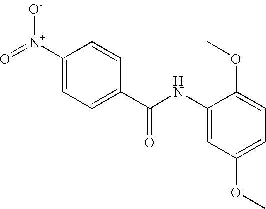 | 169945-47-5 |
| 301 | 3-Methyl-N-phenyl-benzamide | 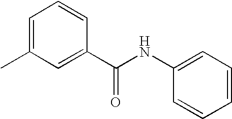 | 23099-05-0 |
| 302 | 4-Bromo-N-(3-methoxy-phenyl)-benzamide | 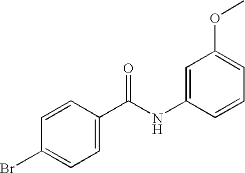 | 313268-57-4 |
| 303 | N-(3-Methoxy-phenyl)-3-nitro-benzamide | 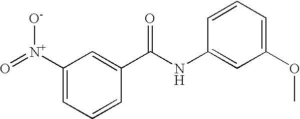 | 107915-06-0 |
| 304 | Naphthalene-2-carboxylicacid (3-methoxy-phenyl)-amide | 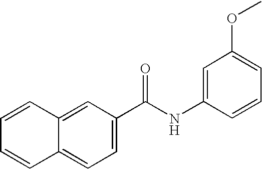 | 88606-02-4 |
| 305 | 4-Chloro-N-(3-methoxy-phenyl)-benzamide | 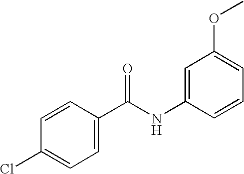 | 42182-03-6 |
| 306 | 3,4-Dimethoxy-N-(3-methoxy-phenyl)-benzamide | 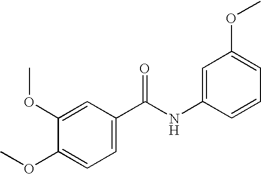 | 1718-91-8 |
| 307 | 3-Methoxy-N-(3-methoxy-phenyl)-benzamide | 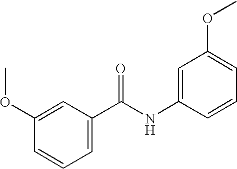 | 97492-32-5 |
| 308 | 4-Chloro-N-(2,5-dimethoxy-phenyl)-benzamide | 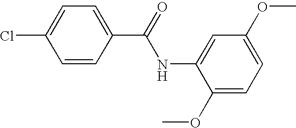 | 262436-40-8 |
| 309 | N-(3,4-Dichloro-phenyl)-benzamide | 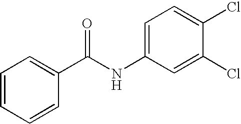 | 10286-75-6 |
| 310 | 2-Chloro-N-(3,4-dichloro-phenyl)-benzamide | 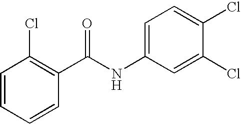 | 7017-22-3 |
| 311 | N-(2,5-Dimethoxy-phenyl)-3-nitro-benzamide | 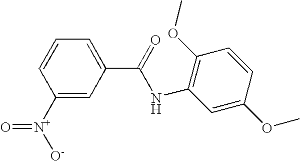 | 142000-62-2 |
| 312 | 3-Nitro-N-phenyl-benzamide | 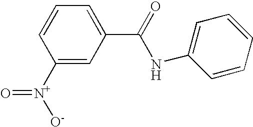 | 2243-73-4 |
| 313 | N-p-Tolyl-benzamide | 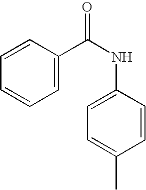 | 582-78-5 |
| 314 | N-(3-Methoxy-phenyl)-4-methyl-benzamide | 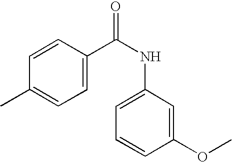 | 101078-45-9 |
| 315 | N-(3-Chloro-phenyl)-benzamide | 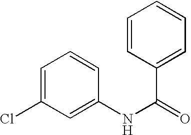 | 6004-21-3 |
| 316 | 4-tert!-Butyl-N-phenyl-benzamide | 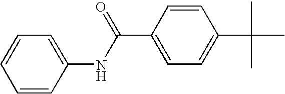 | 65861-72-5 |
| 317 | N-Phenyl-4-trifluoromethyl-benzamide | 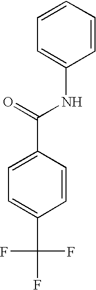 | 347-80-8 |
| 318 | 3-(4-bromo-phenylsulfamoyl)-N-phenyl-benzamide | 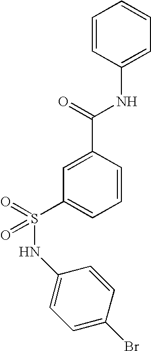 | 347-80-8 |
| 319 | 3,4-Dichloro-N-phenyl-benzamide | 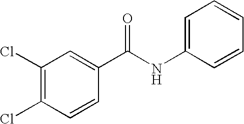 | 6043-42-1 |
| 320 | 4-Butoxy-N-phenyl-benzamide | 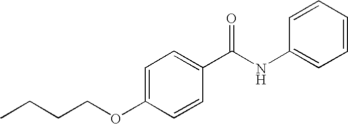 | 33707-64-1 |
| 321 | 3,4-Dimethyl-N-phenyl-benzamide | 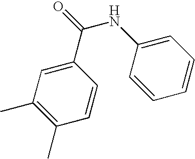 | 164290-86-2 |
| 322 | 2,4-Dichloro-N-phenyl-benzamide | 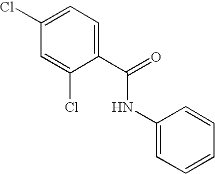 | 6043-39-6 |
| 323 | N-(3,4-Dichloro-phenyl)-3-fluoro-benzamide | 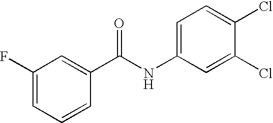 | 58954-98-6 |
| 324 | 4-Nitro-N-phenyl-3-trifluoromethyl-benzamide | 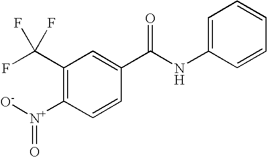 | 478797-87-4 |
| 325 | 4-Isopropyl-N-phenyl-benzamide | 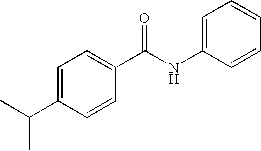 | 15088-90-1 |
| 326 | 3,4-Dichloro-N-p-tolyl-benzamide | 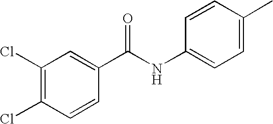 | 6043-43-2 |
| 327 | 3,4-Dichloro-N-methyl-N-phenyl-benzamide | 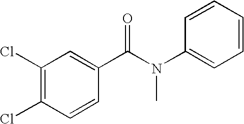 | 26094-80-4 |
| 328 | N-(3-Chloro-phenyl)-4-methyl-benzamide | 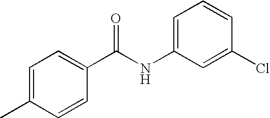 | 81636-13-7 |
| 329 | 3-Methyl-N-p-tolyl-benzamide | 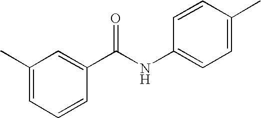 | 97405-27-1 |
| 330 | N-(4-Methoxy-phenyl)-3-methyl-benzamide | 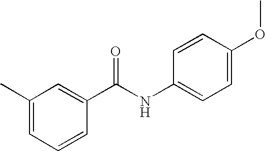 | 313367-17-8 |
| 331 | 2,4-Dichloro-N-(3,4-dichloro-phenyl)-benzamide | 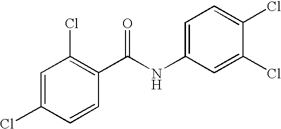 | 83426-48-6 |
| 332 | N-(3,4-Dichloro-phenyl)-2-methyl-benzamide | 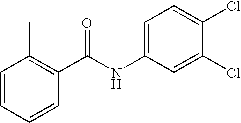 | 71267-58-8 |
| 333 | 6-Chloro-N-(3-methoxy-phenyl)-nicotinamide | 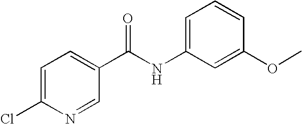 | 224817-08-7 |

1. A method for treating a CNS disorder selected from the group consisting of depression, psychosis, Parkinson's disease, schizophrenia, anxiety and attention deficit hyperactivity disorder (ADHD) which comprises administering to an individual a therapeutically effective amount of a compound of formula I 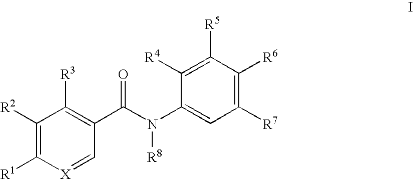
wherein
R¹ is hydrogen, halogen, lower alkyl, lower alkyl substituted by halogen, cycloalkyl, lower alkoxy, NO₂, —(CH₂)ₒS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O)O-lower alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl; or
R¹ and R² together with the corresponding C-atoms form a ring comprising —CH═CH—CH═CH-or —S—(CH₂)₄—;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR′R″, oxazol-5-yl or halogen;
R⁵ and R⁶ form together with the corresponding C-atoms a ring comprising —CH═CH—CH═CH—;
R⁸ is hydrogen or lower alkyl;
X is —C(R⁹)═ or —N═;
R⁹ is hydrogen, lower alkoxy, NO₂ or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ—O-lower alkyl;
n is 0, 1, 2 or 3, and
o is 0 or 1,
or a pharmaceutically acceptable acid addition salt thereof.

2. The method of claim 1, wherein X is —C(R⁹)═.

3. The method of claim 2, wherein R¹ is morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O)O-lower alkyl, 1,1-dioxoisothiazolidin-2-yl, azepan-1-yl, azetidin-1-yl or NR′R″.

4. The method of claim 3, wherein the compound administered is selected from the group consisting of
N-(3-methoxy-phenyl)-4-(4-methyl-piperidin-1-yl)-3-nitro-benzamide,
N-(3-methoxy-phenyl)-3-nitro-4-propylamino-benzamide,
4-benzylamino-N-(3-methoxy-phenyl)-3-nitro-benzamide,
4-ethylamino-N-(3-methoxy-phenyl)-3-nitro-benzamide,
4-isopropylamino-N-(3-methoxy-phenyl)-3-nitro-benzamide,
4-azetidin-1-yl-N-(3-methoxy-phenyl)-3-nitro-benzamide,
N-(3-methoxy-phenyl)-3-nitro-4-pyrrolidin-1-yl-benzamide,
N-(3-methoxy-phenyl)-3-nitro-4-piperidin-1-yl-benzamide,
N-(3-methoxy-phenyl)-3-nitro-4-phenylamino-benzamide,
N-(3-methoxy-phenyl)-4-pyrrolidin-1-yl-3-trifluoromethyl-benzamide,
4-(2-methoxy-ethylamino)-N-(3-methoxy-phenyl)-3-trifluoromethyl-benzamide,
4-azetidin-1-yl-N-(3-methoxy-phenyl)-3-trifluoromethyl-benzamide, and
N-(3-methoxy-phenyl)-4-piperidin-1-yl-3-trifluoromethyl-benzamide.

5. The method of claim 3, wherein the compound administered is selected from the group consisting of
N-(3-methoxy-phenyl)-4-(4-methyl-piperidin-1-yl)-3-trifluoromethyl-benzamide,
N-(3-methoxy-phenyl)-4-propylamino-3-trifluoromethyl-benzamide,
4-butylamino-N-(3-methoxy-phenyl)-3-trifluoromethyl-benzamide,
4-benzylamino-N-(3-methoxy-phenyl)-3-trifluoromethyl-benzamide,
4-ethylamino-N-(3-methoxy-phenyl)-3-trifluoromethyl-benzamide,
4-isopropylamino-N-(3-methoxy-phenyl)-3-trifluoromethyl-benzamide,
N-(3-methoxy-phenyl)-4-morpholin-4-yl-3-trifluoromethyl-benzamide,
N-(3-ethyl-phenyl)-4-pyrrolidin-1-yl-3-trifluoromethyl-benzamide,
N-(3-ethoxy-phenyl)-4-pyrrolidin-1-yl-3-trifluoromethyl-benzamide,
N-(3-isopropyl-phenyl)-4-pyrrolidin-1-yl-3-trifluoromethyl-benzamide,
N-(3-isopropoxy-phenyl)-4-pyrrolidin-1-yl-3-trifluoromethyl-benzamide, and
N-(3-acetyl-phenyl)-4-pyrrolidin-1-yl-3-trifluoromethyl-benzamide.

6. The method of claim 3, wherein the compound administered is selected from the group consisting of
N-(3-fluoro-phenyl)-4-pyrrolidin-1-yl-3-trifluoromethyl-benzamide,
N-(3-chloro-phenyl)-4-pyrrolidin-1-yl-3-trifluoromethyl-benzamide,
N-(3-bromo-phenyl)-4-pyrrolidin-1-yl-3-trifluoromethyl-benzamide,
4-pyrrolidin-1-yl-N-m-tolyl-3-trifluoromethyl-benzamide,
N-(3-difluoromethoxy-phenyl)-4-pyrrolidin-1-yl-3-trifluoromethyl-benzamide,
4-pyrrolidin-1-yl-N-[3-(1,1,2,2-tetrafluoro-ethoxy)-phenyl]-3-trifluoromethyl-benzamide,
(rac,meso)-4-(3,5-dimethyl-piperidin-1-yl)-N-(3-methoxy-phenyl)-3-trifluoromethyl-benzamide,
4-azepan-1-yl-N-(3-methoxy-phenyl)-3-trifluoromethyl-benzamide,
4-(4-cyano-piperidin-1-yl)-N-(3-methoxy-phenyl)-3-trifluoromethyl-benzamide, and
N-(3-methoxy-phenyl)-3-trifluoromethyl-4-(4-trifluoromethyl-piperidin-1-yl)-benzamide.

7. The method of claim 2, wherein R¹ is halogen.

8. The method of claim 7, wherein the compound administered is selected from the group consisting of
4-chloro-N-phenyl-3-trifluoromethyl-benzamide,
4-chloro-N-(3-methoxy-phenyl)-3-nitro-benzamide,
4-bromo-N-(3-methoxy-phenyl)-3-nitro-benzamide,
3-chloro-4-fluoro-N-(3-methoxy-phenyl)-benzamide,
3-bromo-4-fluoro-N-(3-methoxy-phenyl)-benzamide,
4-fluoro-N-(3-methoxy-phenyl)-3-trifluoromethyl-benzamide,
4-fluoro-N-(3-methoxy-phenyl)-3-nitro-benzamide,
3,4-dichloro-N-[3-(2,5-dimethyl-imidazol-1-ylmethyl)-phenyl]-benzamide,
3,4-dichloro-N-phenyl-benzamide,
4-chloro-N-(3-methoxy-phenyl)-3-trifluoromethyl-benzamide,
3,4-dichloro-N-phenyl-benzamide,
3,3′,4-trichlorobenzanilide, and
3,4-dichloro-N-(3-chloro-phenyl)-benzamide.

9. The method of claim 2, wherein R¹ is nitro.

10. The method of claim 9, wherein the compound administered is selected from the group consisting of
3-trifluoromethyl-4-nitro-N-phenyl-benzamide and
4-nitro-N-phenyl-3-trifluoromethyl-benzamide.

11. The method of claim 2 wherein R¹ is hydrogen.

12. The method of claim 11, wherein the compound administered is N-(3,4-dichloro-phenyl)-3-methyl-benzamide.

13. The method of claim 1, wherein X is —N═.

14. The method of claim 13, wherein and R¹ is morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O)O-lower alkyl, 1,1-dioxoisothiazolidin-2-yl, azepan-1-yl, azetidin-1-yl or NR′R″.

15. The method of claim 14, wherein the compound administered is selected from the group consisting of
N-(3-chloro-phenyl)-6-piperazin-1-yl-nicotinamide,
N-(3-chloro-phenyl)-6-(4-methyl-piperazin-1-yl)-nicotinamide,
5-chloro-N-(3-chloro-phenyl)-6-methylamino-nicotinamide,
5-chloro-N-(3-chloro-phenyl)-6-isopropylamino-nicotinamide,
5-chloro-N-(3-chloro-phenyl)-6-(2-methoxy-ethylamino)-nicotinamide,
5-chloro-N-(3-chloro-phenyl)-6-pyrrolidin-1-yl-nicotinamide,
3′-chloro-4-methyl-3,4,5,6-tetrahydro-2H-[1,2′]bipyridinyl-5′-carboxylic acid (3-chloro-phenyl)-amide,
5-chloro-N-(3-chloro-phenyl)-6-ethylamino-nicotinamide,
5-chloro-N-(3-chloro-phenyl)-6-propylamino-nicotinamide,
6-butylamino-5-chloro-N-(3-chloro-phenyl)-nicotinamide, and
6-azetidin-1-yl-5-chloro-N-(3-chloro-phenyl)-nicotinamide.

16. The method of claim 14, wherein the compound administered is selected from the group consisting of
3′-chloro-3,4,5,6-tetrahydro-2H-[1,2′]bipyridinyl-5′-carboxylic acid (3-chloro-phenyl)amide,
5-chloro-N-(3-chloro-phenyl)-6-(4-methyl-piperazin-1-yl)-nicotinamide,
N-(3-methoxy-phenyl)-6-pyrrolidin-1-yl-5-trifluoromethyl-nicotinamide,
6-benzylamino-N-(3-methoxy-phenyl)-5-trifluoromethyl-nicotinamide,
6-isopropylamino-N-(3-methoxy-phenyl)-5-trifluoromethyl-nicotinamide,
4-methyl-3′-trifluoromethyl-3,4,5,6-tetrahydro-2H-[1,2′]bipyridinyl-5′-carboxylic acid (3-methoxy-phenyl)-amide,
5-chloro-N-(3-methoxy-phenyl)-6-pyrrolidin-1-yl-nicotinamide,
3′-chloro-4-methyl-3,4,5,6-tetrahydro-2H-[1,2′]bipyridinyl-5′-carboxylic acid (3-methoxy-phenyl)-amide,
6-butylamino-5-chloro-N-(3-methoxy-phenyl)-nicotinamide, and
5-chloro-N-(3-chloro-phenyl)-6-piperazin-1-yl-nicotinamide.

17. The method of claim 13, wherein R¹ is halogen.

18. The method of claim 17, wherein the compound administered is selected from the group consisting of
5,6-dichloro-N-(3-chloro-phenyl)-nicotinamide,
5,6-dichloro-N-(3-methoxy-phenyl)-nicotinamide, and
6-chloro-N-(3-methoxy-phenyl)-5-trifluoromethyl-nicotinamide.

19. A compound of formula IA 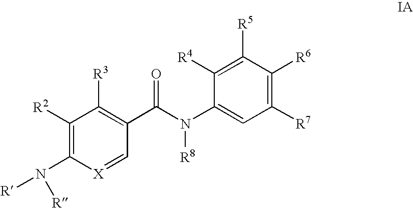
wherein
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR′R″, oxazol-5-yl or halogen; or
R⁵ and R⁶ together with the corresponding C-atoms form a ring comprising —CH═CH—CH═CH—;
R⁸ is hydrogen or lower alkyl;
X is —C(R⁹)═ or —N═;
R⁹ is hydrogen, lower alkoxy, NO₂ or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ—O-lower alkyl;
n is 0, 1, 2 or 3, and
o is 0 or 1,
or a pharmaceutically acceptable acid addition salt thereof; with the exception of
4-diethylamino-N-phenyl-benzamide
4-acetylamino-3-nitro-N-phenyl-benzamide and
4-dimethylamino-N-phenyl-benzamide.

20. The compound of claim 19, selected from the group consisting of
N-(3-methoxy-phenyl)-3-nitro-4-propylamino-benzamide,
4-benzylamino-N-(3-methoxy-phenyl)-3-nitro-benzamide,
4-ethylamino-N-(3-methoxy-phenyl)-3-nitro-benzamide,
4-isopropylamino-N-(3-methoxy-phenyl)-3-nitro-benzamide,
N-(3-methoxy-phenyl)-3-nitro-4-phenylamino-benzamide,
4-(2-methoxy-ethylamino)-N-(3-methoxy-phenyl)-3-trifluoromethyl-benzamide,
N-(3-methoxy-phenyl)-4-propylamino-3-trifluoromethyl-benzamide,
4-butylamino-N-(3-methoxy-phenyl)-3-trifluoromethyl-benzamide,
4-benzylamino-N-(3-methoxy-phenyl)-3-trifluoromethyl-benzamide,
4-ethylamino-N-(3-methoxy-phenyl)-3-trifluoromethyl-benzamide,
4-isopropylamino-N-(3-methoxy-phenyl)-3-trifluoromethyl-benzamide,
6-benzylamino-N-(3-methoxy-phenyl)-5-trifluoromethyl-nicotinamide,
6-isopropylamino-N-(3-methoxy-phenyl)-5-trifluoromethyl-nicotinamide, and
6-butylamino-5-chloro-N-(3-methoxy-phenyl)-nicotinamide.

21. A compound of formula IB 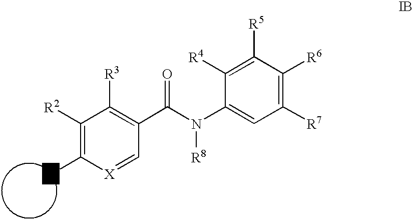
wherein 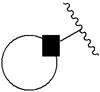
is a cyclic amine group, selected from morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O) O-lower alkyl, 1,1-dioxoisothiazolidin-1-yl, azepan-1-yl and azetidin-1-yl;
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR′R″, oxazol-5-yl or halogen; or
R⁵ and R⁶ together with the corresponding C-atoms form a ring comprising —CH═CH—CH═CH—;
R⁸ is hydrogen or lower alkyl;
X is —C(R⁹)═ or —N═;
R⁹ is hydrogen, lower alkoxy, NO₂ or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ—O-lower alkyl;
n is 0, 1, 2 or 3, and
o is 0 or 1,
or a pharmaceutically acceptable acid addition salt thereof.

22. The compound of claim 21, selected from the group consisting of
N-(3-methoxy-phenyl)-4-(4-methyl-piperidin-1-yl)-3-nitro-benzamide,
4-azetidin-1-yl-N-(3-methoxy-phenyl)-3-nitro-benzamide,
N-(3-methoxy-phenyl)-3-nitro-4-pyrrolidin-1-yl-benzamide,
N-(3-methoxy-phenyl)-3-nitro-4-piperidin-1-yl-benzamide,
N-(3-methoxy-phenyl)-4-pyrrolidin-1-yl-3-trifluoromethyl-benzamide,
4-azetidin-1-yl-N-(3-methoxy-phenyl)-3-trifluoromethyl-benzamide,
N-(3-methoxy-phenyl)-4-piperidin-1-yl-3-trifluoromethyl-benzamide,
N-(3-methoxy-phenyl)-4-(4-methyl-piperidin-1-yl)-3-trifluoromethyl-benzamide,
N-(3-methoxy-phenyl)-4-morpholin-4-yl-3-trifluoromethyl-benzamide, and
N-(3-ethyl-phenyl)-4-pyrrolidin-1-yl-3-trifluoromethyl-benzamide.

23. The compound of claim 21, selected from the group consisting of
N-(3-ethoxy-phenyl)-4-pyrrolidin-1-yl-3-trifluoromethyl-benzamide,
N-(3-isopropyl-phenyl)-4-pyrrolidin-1-yl-3-trifluoromethyl-benzamide,
N-(3-isopropoxy-phenyl)-4-pyrrolidin-1-yl-3-trifluoromethyl-benzamide,
N-(3-acetyl-phenyl)-4-pyrrolidin-1-yl-3-trifluoromethyl-benzamide,
N-(3-fluoro-phenyl)-4-pyrrolidin-1-yl-3-trifluoromethyl-benzamide,
N-(3-chloro-phenyl)-4-pyrrolidin-1-yl-3-trifluoromethyl-benzamide,
N-(3-bromo-phenyl)-4-pyrrolidin-1-yl-3-trifluoromethyl-benzamide,
4-pyrrolidin-1-yl-N-m-tolyl-3-trifluoromethyl-benzamide,
N-(3-difluoromethoxy-phenyl)-4-pyrrolidin-1-yl-3-trifluoromethyl-benzamide, and
4-pyrrolidin-1-yl-N-[3-(1,1,2,2-tetrafluoro-ethoxy)-phenyl]-3-trifluoromethyl-benzamide.

24. The compound of claim 21, selected from the group consisting of
(rac,meso)-4-(3,5-dimethyl-piperidin-1-yl)-N-(3-methoxy-phenyl)-3-trifluoromethyl-benzamide,
4-azepan-1-yl-N-(3-methoxy-phenyl)-3-trifluoromethyl-benzamide,
4-(4-cyano-piperidin-1-yl)-N-(3-methoxy-phenyl)-3-trifluoromethyl-benzamide,
N-(3-methoxy-phenyl)-3-trifluoromethyl-4-(4-trifluoromethyl-piperidin-1-yl)-benzamide,
4-methyl-3′-trifluoromethyl-3,4,5,6-tetrahydro-2H-[1,2′]bipyridinyl-5′-carboxylic acid (3-methoxy-phenyl)-amide,
5-chloro-N-(3-methoxy-phenyl)-6-pyrrolidin-1-yl-nicotinamide,
3′-chloro-3,4,5,6-tetrahydro-2H-[1,2′]bipyridinyl-5′-carboxylic acid (3-methoxy-phenyl)-amide,
3′-chloro-4-methyl-3,4,5,6-tetrahydro-2H-[1,2′]bipyridinyl-5′-carboxylic acid (3-methoxy-phenyl)-amide, and
5-chloro-N-(3-chloro-phenyl)-6-piperazin-1-yl-nicotinamide.

25. A pharmaceutical composition comprising a therapeutically effective amount of a compound of formula IA 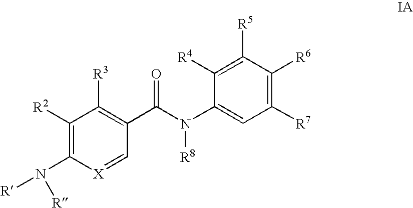
wherein
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR═R|, oxazol-5-yl or halogen; or
R⁵ and R⁶ together with the corresponding C-atoms form a ring comprising —CH═CH—CH═CH—;
R⁸ is hydrogen or lower alkyl;
X is —C(R⁹)═ or —N═;
R⁹ is hydrogen, lower alkoxy, NO₂ or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ-O-lower alkyl;
n is 0, 1, 2 or 3, and
o is 0 or 1,
or a pharmaceutically acceptable acid addition salt thereof; with the exception of
4-diethylamino-N-phenyl-benzamide
4-acetylamino-3-nitro-N-phenyl-benzamide and
4-dimethylamino-N-phenyl-benzamide
and a pharmaceutically acceptable carrier.

26. A pharmaceutical composition comprising a therapeutically effective amount of a compound of formula IB 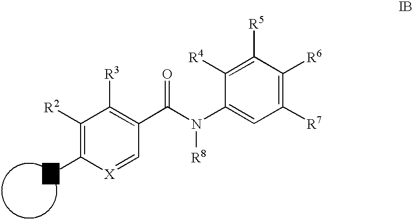
wherein 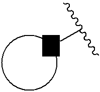
is a cyclic amine group, selected from morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin-1-yl, piperazin-1-yl, 4-methyl-piperazin-1-yl, 3,5-dimethyl-piperidin-1-yl, piperazin-1-yl substituted by C(O) O-lower alkyl, 1,1-dioxoisothiazolidin-1-yl, azepan-1-yl and azetidin-1-yl;
R² is hydrogen, halogen, lower alkyl, lower alkoxy, lower alkyl substituted by halogen, lower alkoxy substituted by halogen, cyano, NO₂, —(CH₂)ₒS(O)₂R, —OS(O)₂NR′R″, lower alkyl-O—C(═CH₂)—, —C(O)-lower alkyl, tetrahydro-furan-2-yl, morpholin-4-yl, pyrazol-1-yl, or —OC(O)-lower alkyl;
R³ is hydrogen, halogen, lower alkyl or lower alkoxy;
R⁴ is hydrogen, lower alkoxy or halogen;
R⁵ and R⁷ are each independently hydrogen, halogen, lower alkyl, lower alkoxy, NO₂, cyano, lower alkyl substituted by halogen, lower alkoxy substituted by halogen, phenyl, O-phenyl, —(CH₂)ₒS(O)₂R, NHC(O)-lower alkyl, C(O)-lower alkyl, C(O)O-lower alkyl or 2,5-dimethyl-imidazol-1-yl-methyl;
R⁶ is hydrogen, lower alkoxy, cyano, nitro, lower alkyl, phenyl, lower alkyl substituted by halogen, lower alkoxy substituted by halogen, C(O)O-lower alkyl, C(O)O—(CH₂)₂—NR′R″, oxazol-5-yl or halogen; or
R⁵ and R⁶ together with the corresponding C-atoms form a ring comprising —CH═CH—CH═CH—;
R⁸ is hydrogen or lower alkyl;
X is —C(R⁹)═ or —N═;
R⁹ is hydrogen, lower alkoxy, NO₂ or halogen;
R is lower alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl;
R′ and R″ are each independently hydrogen, lower alkyl, (CH₂)ₙ-4-methylpiperidin-1-yl, (CH₂)ₙ—C(O)-lower alkyl, (CH₂)ₙ-phenyl optionally substituted by halogen or (CH₂)ₙ—O-lower alkyl;
n is 0, 1, 2 or 3, and
o is 0 or 1,
or a pharmaceutically acceptable acid addition salt thereof and a pharmaceutically acceptable carrier.
